SEQUENCE LISTING (11 sequences)

SEQ ID NO: 1 - protein, 8 aa, Homo sapiens
  misc_feature (7)..(7): Xaa can be any naturally occurring amino acid
DRFYIPXR

SEQ ID NO: 2 - protein, 22 aa, Homo sapiens
CFSKTRSTKESVSVLNLFTRIR

SEQ ID NO: 3 - protein, 20 aa, Homo sapiens
CFSKTRSTKESVSVLNLFTR

SEQ ID NO: 4 - RNA, 19 nt, Homo sapiens
gaaaggcugucauguauug

SEQ ID NO: 5 - RNA, 19 nt, Homo sapiens
caacagacacuuaauagua

SEQ ID NO: 6 - RNA, 19 nt, Homo sapiens
cgaagugucucuguaauua

SEQ ID NO: 7 - RNA, 19 nt, Homo sapiens
guacgaagugucucuguaa

SEQ ID NO: 8 - DNA, 21 bp, Homo sapiens
ggaacaacugaaagauuggtt

SEQ ID NO: 9 - RNA, 21 nt, Homo sapiens
uaaauuaagccucgguugauu

SEQ ID NO: 10 - RNA, 21 nt, Homo sapiens
ggaaauagccgagagguaauu

SEQ ID NO: 11 - RNA, 21 nt, Homo sapiens
ggaagaagaucuagauguauu

(19) United States
(12) Patent Application Publication
(10) Pub. No.: US 2013/0230458 A1
(43) Pub. Date: Sep. 5, 2013

(54) Cell Permeable Inhibitors of Anaphase Promoting Complex

(71) Applicants: Randall King, Newton, MA (US); Xing Zeng, Jamaica Plain, MA (US)

(72) Inventors: Randall King, Newton, MA (US); Xing Zeng, Jamaica Plain, MA (US)

(73) Assignee: PRESIDENT AND FELLOWS OF HARVARD COLLEGE, Cambridge, MA (US)

(21) Appl. No.: 13/819,957

(22) PCT Filed: Sep. 1, 2011
(86) PCT No.: PCT/US2011/050203

§ 371 Date: May 23, 2013

Related U.S. Application Data
(60) Provisional application No. 61/379,111, filed on Sep. 1, 2010.

(51) Int. Cl. A61K 31/325; A61K 31/337; A61K 45/06; A61K 51/02

(52) U.S. Cl. CPC A61K 31/325; A61K 51/025; A61K 31/337; A61K 45/06
USPC 424/1.61; 514/449; 514/483; 560/13

(57) ABSTRACT

The invention provides compositions and methods for treating cell cycle disorders. Compositions of the invention include proTAME, a prodrug analog of TAME, and, optionally, one or more therapeutic agents.

### RELATED APPLICATIONS

This application claims the benefit of provisional application U.S. Ser. No. 61/379,111, filed Sep. 1, 2010, the contents which are herein incorporated by reference in their entirety.

### GOVERNMENT SUPPORT

This invention was made with U.S. Government support under National Institutes of Health grant RO1 GM66492. The Government has certain rights in the invention.

### INCORPORATION OF SEQUENCE LISTING

The contents of the text file named “29297-060001WO_ST25.txt,” which was created on Sep. 1, 2011 and is 2.21 KB in size, are hereby incorporated by reference in their entirety.

### FIELD OF THE INVENTION

This invention relates generally to the fields of oncology and disorders associated with cell division.

### BACKGROUND OF THE INVENTION

Conventional antiproliferative agents used in the treatment of cancer are generally grouped as compounds which affect the integrity of nucleic acid polymers, e.g., by binding, alkylating, inducing strand breaks, intercalating between base pairs or affecting enzymes which maintain the integrity and function of DNA and RNA, and compounds that bind to proteins to inhibit enzymatic action (e.g., antimetabolites) or the function of structural proteins necessary for cellular integrity (e.g., antitubulin agents). Other antiproliferative drugs include those that block steroid hormone action for the treatment of hormone-dependent cancer, photochemically activated agents, radiation sensitizers, and protectors. Many of these agents are associated with adverse side effects.

### SUMMARY OF THE INVENTION

The invention encompasses Anaphase-Promoting Complex/Cyclosome (APC) inhibitors as anticancer agents and improves upon the success of known anti-cancer agents, such as taxanes, in treating a wide variety of different cancers. Taxanes bind microtubules and activate the spindle checkpoint, which can arrest cells in mitosis and induce cell death. However, taxane-treated cells can exit mitosis before dying, through a process of “mitotic slippage” that results from incomplete inhibition of the APC by the Spindle Assembly Checkpoint (SAC). Variability in the relative rates of mitotic slippage compared to mitotic cell death may explain the variability in sensitivity of cell lines and tumors to taxanes and mitotic kinesin inhibitors.

Direct inhibition of APC is a more effective way of inducing mitotic arrest than stimulating the SAC. For example, knockdown of Cdc20, which is an activator of APC, by RNAi induces prolongs mitotic arrest and cell death, but only if Cdc20 is reduced to very low levels. This approach to inducing cell death shows much less variability across different cancer cell lines, compared to taxane treatment. Furthermore, studies in mice similarly suggest that elimination of Cdc20 function can lead to very effective tumor regression in animals.

The small molecules of the invention inhibit APC^{Cdc20} by pharmacologic approaches to recapitulate the effects of Cdc20 knockdown or knockout. The cell-permeable prodrug of TAME, proTAME is unexpectedly effective at inducing mitotic arrest in cells. One explanation for the effectiveness of proTAME is that proTAME interferes with the process of SAC inactivation. For example, mitotic arrest induced by taxol requires ongoing protein synthesis in mitosis, because APC-dependent proteolysis is not fully inhibited by the SAC. In contrast, proTAME causes more profound APC inhibition, such that mitotic arrest is independent of protein synthesis. These discoveries explain why direct APC inhibition is more effective at inducing prolonged mitotic arrest and cell death than microtubule inhibition.

The invention provides a composition including a prodrug of tosyl-L-arginine methylester (TAME). The prodrug diffuses across the plasma membrane of a cell and decreases cell proliferation by inhibiting mitosis. Once inside the cell, the prodrug is processed to yield the active agent, TAME. The prodrug is formulated into a pharmaceutical composition which includes the prodrug and a physiologically acceptable excipient. Preferably, the prodrug is a TAME derivative in which a guanidino group is linked to a protecting group. The protecting group is an enzyme-cleavable group such as a carbamate group that is cleaved by an intracellular enzyme. The guianidino substituent of TAME occurs in the arginine residue. An exemplary prodrug is an esterase-activatable N,N′-bis (acyloxymethyl carbamate) derivative of the parent molecule, TAME.

In certain embodiments, proTAME, and its parent molecule (TAME), once inside the cell, contact a component of a tetratricopeptide repeats (TPR) subcomplex of an APC. Exemplary components of a TPR subcomplex include, but are not limited to, APC3/Cdc27, APC6, APC7, or APC8. In a preferred embodiment, proTAME/TAME blocks the interaction of Cdc20 with APC through the interaction of the IR tail of Cdc20 with APC3.

The phospholipid membrane of cells limits the permeation of molecules into a cell. The membrane acts as a barrier to passive diffusion of water-soluble molecules, and substances that dissolve in lipids pass more easily into the cell. The prodrugs described herein are lipophilic, uncharged, esterase-activatable prodrugs of guanidine containing molecules. The prodrug has improved cell permeability, and once inside the cell, is converted to active TAME by esterase(s). The prodrug is characterized as at least 10%, 20%, 50%, 2-fold, 5-fold, 20-fold or more membrane-permeable (e.g., eukaryotic cell membrane permeable) compared to the parent drug.

A “prodrug” is a molecule which is converted to a therapeutically active molecule after administration. For example, prodrug (e.g., proTAME) converted to TAME after gaining access into a mammalian cell. Conversion of the prodrug to the active drug may occur by hydrolysis of an ester group or some other biologically labile group. Generally, but not necessarily, a prodrug is inactive or less active than the therapeutically active molecule to which it is converted. The prodrug is characterized by a protecting group that is associated with or linked to an inhibitor of the anaphase promoting complex (APC). The presence of the group, e.g., a carbamate group on a guanidine substituent of arginine increases cellular uptake or transport across a cellular membrane (e.g. plasma membrane or nuclear membrane). The prodrug has additional advantages such as improved solubility, oral absorption, cellular targeting or specificity, stability, half-life, or blood-brain barrier permeability of the inhibitory molecule. Alternatively, or in addition, prodrugs decrease the degradation, immunogenicity, toxicity, or required dosage compared to the parent molecule.

Also within the invention is a method for manufacturing an anti-proliferative agent such as a cell-permeable inhibitor of APC by introducing an amino protecting group, e.g., a carbamate protecting group, onto a guanidino substituent of an arginine residue of TAME. The carbamate protecting group is introduced by contacting said TAME with N,N′-bis (benzyloxycarbonyl)-S-methylisourea.

In a preferred embodiment of the invention, the molecule is proTAME:

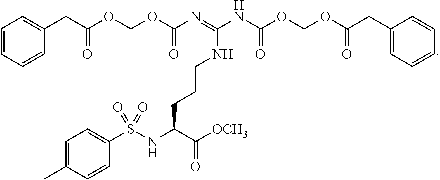 [SMILES: COC(=O)[C@H](CCCN/C(=N\C(=O)OCOC(=O)Cc1ccccc1)NC(=O)OCOC(=O)Cc1ccccc1)NS(=O)(=O)c1ccc(C)cc1]

ProTAME, or a pharmaceutically acceptable salt, ester, tautomer, or solvate thereof, may be included in a composition or pharmaceutical composition of the invention.

In certain embodiments, compositions and compounds contact a component of a tetratricopeptide repeats (TPR) subcomplex of an APC. Exemplary components of a TPR subcomplex include, but are not limited to, APC3/Cdc27, APC6, APC7, or APC8.

In a further aspect of the invention, compositions and compounds induce a cell cycle checkpoint. In certain embodiments of the invention, the cell cycle checkpoint is the spindle assembly checkpoint (SAC).

The invention also provides a method for inhibiting a ubiquitination activity of an APC including administering an effective amount of proTAME to a cell to inhibit the degradation of a substrate of an APC.

The invention further provides a method for inhibiting a ubiquitination activity of an APC including administering an effective amount of proTAME to a cell to induce a cell cycle checkpoint. In certain embodiments of the invention, the cell cycle checkpoint is the spindle assembly checkpoint (SAC). ProTAME arrests cells in mitosis via (a) direct APC inhibition, and/or (b) by preventing inactivation of the SAC.

The invention provides a method of treating a cell cycle disorder, including administering to a subject in need thereof an effective amount of a pharmaceutical composition described herein, wherein the composition inhibits an activity of an anaphase promoting complex (APC).

The invention provides a method of treating a cell cycle disorder, including administering to a subject in need thereof an effective amount of a pharmaceutical composition described herein, wherein the composition activates a cell cycle checkpoint. In certain embodiments of the invention, the cell cycle checkpoint is the spindle assembly checkpoint (SAC).

The invention provides a method of arresting cancer cell growth, including contacting the cell with composition containing a spindle assembly checkpoint activator and a proTAME composition, thereby inducing mitotic arrest or cell death.

The invention also provides a method of arresting cancer cell growth, including contacting the cell with a composition containing an inhibitor of proteasome-dependent degradation and a proTAME composition, thereby inducing mitotic arrest or cell death.

The contacting step of these methods is performed in vivo, in vitro, or ex vivo. ProTAME increases the median or mean mitotic duration of the cancer cell. Furthermore, proTAME induces death of the cancer cell.

“Isomerism” describes compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four non-identical substituents is termed a “chiral center”.

“Chiral isomer” describes a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center.

Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” of a compound means a salt that is pharmaceutically acceptable and that possesses the desired pharmacological activity of the parent compound.

“Pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

All references to pharmaceutically acceptable salts include solvent addition forms (solvates) or crystal forms (polymorphs) as defined herein, of the same salt.

Pharmaceutically acceptable salts of the present invention can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; non-aqueous media like ether, ethyl acetate, ethanol, isopropanol, or acetonitrile can be used. Lists of suitable salts are found in Remington's Pharmaceutical Sciences, 18th ed. (Mack Publishing Company, 1990). For example, salts can include, but are not limited to, the hydrochloride and acetate salts of the aliphatic amine-containing, hydroxylamine-containing, and imine-containing compounds of the present invention.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine. Examples include:

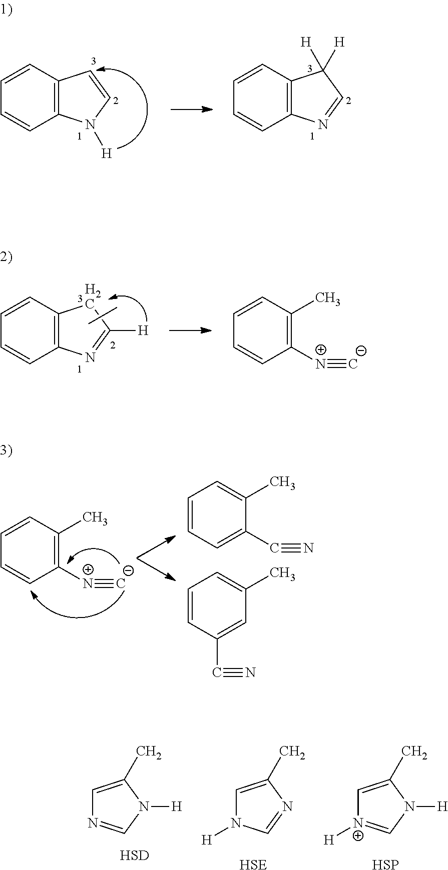

Compounds of the invention may be depicted as different tautomers. When compounds have tautomeric forms, all tautomeric forms are intended to be included in the scope of the present invention, and the naming of the compounds does not exclude any tautomer form.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

The term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

The term “derivative” refers to compounds that have a common core structure, and are substituted with various groups as described herein.

The invention includes all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The invention provides a pharmaceutical composition comprising, consisting essentially of, or consisting of proTAME. Furthermore, the pharmaceutical composition includes a pharmaceutical carrier.

Compositions and formulations of the invention are administered to a cell. The cell is optionally eukaryotic, mammalian, or, preferably, human. In certain aspects of the invention, the compositions and formulations of the invention are administered to the cell in vitro, in vivo, ex vivo, or any combination thereof.

The compositions and molecules of the invention inhibit an activity of an anaphase promoting complex (APC) to arrest mitosis and reduce proliferation of cells such as cancer cells or other aberrantly proliferating cells. The compositions are useful to treat cellular proliferative disorders such as cancers, e.g., skin cancer, virally induced hyperproliferative HPV-papilloma, HSV-shingles, colon cancer, bladder cancer, breast cancer, melanoma, ovarian carcinoma, prostate carcinoma, or lung cancer as well as psoriasis and eczema. The compositions may be also useful in the context of in vitro fertilization, because they enhance the ability of these early embryos to properly segregate DNA during mitosis.

The invention provides a formulation comprising an amount of a prodrug of tosyl-L-arginine methylester (TAME) that is sufficient to inhibit the degradation of a substrate of an anaphase-promoting complex/cyclosome (APC) for arresting the mitotic cycle of a cell.

Moreover, the invention provides a formulation comprising an amount of a prodrug of tosyl-L-arginine methylester (TAME) that is sufficient to inhibit the degradation of a substrate of an anaphase-promoting complex/cyclosome (APC) for arresting the mitotic cycle of a cell, for use in the treatment of a cell proliferative disorder. A preferred cell proliferative disorder is cancer.

The invention provides a method treating a cell proliferative disorder in a subject, comprising administering to the subject an amount of a composition comprising a prodrug of tosyl-L-arginine methylester (TAME) that is sufficient to inhibit the degradation of a substrate of an anaphase-promoting complex/cyclosome (APC), thereby arresting the mitotic cycle of one or more cells in the subject. A preferred cell proliferative disorder is cancer.

These formulations and compositions further include a pharmaceutical carrier.

Optionally, these formulations and compositions include a therapeutic agent. An exemplary therapeutic agent includes, but is not limited to, a chemotherapy, a radiation therapy, an immunotherapy, or a hormone therapy.

Radiation therapy is a radioactive isotope that is either administered alone or conjugated to another molecule, such as a carrier, to administration, and removal of the isotope from a cell, tissue, organ or subject. Exemplary radiation therapies include, but are not limited to, actinium-225 (Ac²²⁵), bismuth-213 (Bi²¹³), boron-10 (B¹⁰)+neutron therapy, holmium-166 (Ho¹⁶⁶), iodine-125 (I¹²⁵), iodine-131 (I¹³³), iridium-192 (Ir¹⁹²), lead-212 (Pb²¹²), lutetium-177 (Lu¹⁷⁷), rhenium-186 (Re¹⁸⁶), samarium-153 (Sm¹⁵³), strontium-89 (Sr⁸⁹), or yttrium-90 (Y⁹⁰).

Immunotherapy is based upon the development of human monoclonal antibody therapies that are either themselves therapeutic, or, alternatively, capable of selectively targeting a cell of interest to aid in the administration of another therapy. For instance, a cell of interest is characterized by a proliferation disorder. Exemplary immunotherapies of the invention include, but are not limited to, rituximab (Rituxan®), trastuzumab (Herceptin®), gemtuzumab ozogamicin (Mylotarg®), alemtuzumab (Campath®), ibritumomab tiuxetan (Zevalin®), tositumomab (Bexxar®), cetuximab (Erbitux®), bevacizumab (Avastin®), panitumumab (Vectibix®), ofatumumab (Arzerra®), denosumab (Xgeva™), ipilimumab (Yervoy™), and brentuximab vedotin (Adcetris™). The hormone therapy is tamoxifen (Nolvadex®), an aromatase, inhibitor, anastrozole (Arimidex®), letrozole (Femara®), or fulvestrant (Fasiodex®).

Chemotherapy includes any pharmacological molecule or composition that mitigates that harm incurred when a normal cell undergoes a transformation into a disease state characterized by aberrant proliferation. Typically, the proliferation of the transformed cell is increased compared to a normal or healthy cell, and, therefore, the chemotherapy is designed to inhibit or prevent cell division. Exemplary chemotherapy agents include, but are not limited to, carboplatin (Paraplatin), cisplatin (Platinol, Platinol-AQ), cyclophosphamide (Cytoxan, Neosar), doxorubicin (Adriamycin), etoposide (VePesid), fluorouracil (5-FU), gemcitabine (Gemzar), irinotecan (Camptosar), methotrexate, (Folex, Mexate, Amethopterin), paclitaxel (Taxol), topotecan (1-Hycamtin), vincristine, (Oncovin, Vincasar PFS), or vinblastine (Velban).

In certain embodiments, the formulations described herein contain a chemotherapy that specifically targets either a cell cycle checkpoint or, specifically, the spindle assembly checkpoint. The chemotherapy may target the APC complex itself.

Alternatively, or in addition, the chemotherapy may confer selectivity for cancer cells upon administration of TAME/proTAME. Selectivity may be achieved by providing a chemotherapeutic agent that protects the normal cells surrounding the target cell by inducing a state of scenescence (or non-division), which allows the TAME/proTAME composition to target only dividing cells. In these embodiments, the chemotherapeutic agent includes, but is not limited to, a nutlin (nutlin-1, nutlin-2, nutlin-3), and sirolimus (rapamycin).

In a specific embodiment, compositions and formulations of the invention include proTAME and a spindle assembly checkpoint activator. Alternatively, or in addition, the spindle assembly checkpoint activator is provided to the same cell or subject, but in a separate composition or formulation. These separate compositions or formulations may comprise the same treatment regime.

In another specific embodiment, compositions and formulations of the invention include proTAME and an inhibitor of proteasome-dependent degredation. Alternatively, or in addition, the inhibitor of proteasome-dependent degredation is provided to the same cell or subject, but in a separate composition or formulation. These separate compositions or formulations may comprise the same treatment regime.

The invention provides methods of treating cell proliferative disorders using compositions and formulations that include proTAME, a spindle assembly checkpoint activator, an inhibitor of proteasome-dependent degredation, or any combination thereof. Preferred spindle assembly checkpoint activators include paclitaxol or Taxol™. A preferred inhibitor of proteasome-dependent degredation is MG132.

The formulations of the invention are administered to any cell, including a eukaryotic, mammalian, or, preferably, human cell. In certain aspects, the cell is characterized by a proliferative disorder. The cell proliferative disorder is, for example, cancer, Castleman Disease, Gestational Trophoblastic Disease, or myelodysplastic syndrome. Nonlimiting examples of cancer include, but are not limited to, adrenal cortical cancer, anal cancer, bile duct cancer, bladder cancer, bone cancer, brain or a nervous system cancer, breast cancer, cervical cancer, colon cancer, rectral cancer, colorectal cancer, endometrial cancer, esophageal cancer, Ewing family of tumor, eye cancer, gallbladder cancer, gastrointestinal carcinoid cancer, gastrointestinal stromal cancer, Hodgkin Disease, intestinal cancer, Kaposi Sarcoma, kidney cancer, large intestine cancer, laryngeal cancer, hypopharyngeal cancer, laryngeal and hypopharyngeal cancer, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, lung cancer, non-small cell lung cancer, small cell lung cancer, lung carcinoid tumor, lymphoma, lymphoma of the skin, malignant mesothelioma, multiple myeloma, nasal cavity cancer, paranasal sinus cancer, nasal cavity and paranasal sinus cancer, nasopharyngeal cancer, neuroblastoma, non-Hodgkin lymphoma, oral cavity cancer, oropharyngeal cancer, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma, adult soft tissue sarcoma, skin cancer, basal cell skin cancer, squamous cell skin cancer, basal and squamous cell skin cancer, melanoma, stomach cancer, small intestine cancer, testicular cancer, thymus cancer, thyroid cancer, uterine sarcoma, uterine cancer, vaginal cancer, vulvar cancer, Waldenstrom Macroglobulinemia, or Wilms Tumor. These cancers may be either primary or metastatic cancer. Moreover, these cancers may occur in either a child or an adult.

The composition containing proTAME and a therapeutic agent are provided to treat a cell proliferative disorder, including cancer. The therapeutic agent may be administered to a cell simultaneously or sequentially with the proTAME composition. In certain embodiments, the composition containing proTAME is provided, contacted, or administered to the cancer cell prior to the therapeutic agent. In other embodiments, the composition containing proTAME is provided, contacted, or administered to the cancer cell at substantially the same time as the therapeutic agent. Substantially the same time may describe a period of 24 hours. In alternative embodiments, the composition containing proTAME is provided, contacted, or administered to the cancer cell at after the therapeutic agent. In all embodiments of these methods, it is understood that when the therapeutic agent is a spindle assembly checkpoint activator or an inhibitor of proteasome-dependent degradation, the therapeutic agent acts in a synergistic manner or demonstrates synergy with the proTAME composition.

Exemplary compositions of the methods described herein optionally include a therapeutic agent. ProTAME enhances mitotic arrest and the amount of cell death induced by a taxane molecule, compound, or drug, e.g. paclitaxol or Taxol™, or by a proteasome inhibitor. Based on the synergistic effect of proTAME and these agents (e.g. taxanes and/or proteasome inhibitors), combination therapies using proTAME together with either a taxane or a proteosome inhibitor (or both) is used to treat or reduce the severity of the cell cycle disorders described herein (e.g. cancer). In one aspect, the second therapeutic agent is a spindle assembly checkpoint activator. A preferred spindle assembly checkpoint activator is paclitaxol or Taxol™. Alternatively, or in addition, the second therapeutic agent is an inhibitor of proteasome-dependent degradation. A preferred inhibitor of proteasome-dependent degradation is MG132.

Taxanes are a family of naturally-occurring or synthetically-produced diterpene compounds that inhibit cell growth and/or division. Exemplary taxanes include paclitaxel (Taxol™), docetaxel (taxotere), abretaxane, taxoprexin, and Xyotax.

Formlations, compositions, compounds, and molecules (drugs, prodrugs) disclosed herein may be mixed or formulated with pharmaceutically acceptable excipients for administration to human or animal subjects. For example, a drug to be administered systemically is formulated as a powder, pill, tablet or the like, or as a solution, emulsion, suspension, aerosol, syrup or elixir suitable for oral or parenteral administration or for inhalation. The compositions or compounds are purified, i.e. isolated from natural sources or chemically synthesized. A purified composition or compound comprises at least 75%, 80%, 90%, or 100% (w/w) of the desired molecule.

Formlations, compositions, compounds, and molecules (drugs, prodrugs) disclosed herein may be administered to a subject locally or systemically by any one of the following routes: topical, intravenous, intraocular, subcutaneous, intraparitoneal, intramuscular, intraspinal, or surgical administration.

The pharmaceutically effective dose depends on the type of disease, the composition used, the route of administration, the individual and physical characteristics of the subject under consideration (for example, age, gender, weight, diet, smoking-habit, exercise-routine, genetic background, medical history, hydration, blood chemistry), concurrent medication, and other factors that those skilled in the medical arts will recognize.

Generally, an amount from about 0.01 mg/kg and 25 mg/kg body weight/day of active ingredients is administered dependent upon potency of the composition containing proTAME. In alternative embodiments dosage ranges include, but are not limited to, 0.01-0.1 mg/kg, 0.01-1 mg/kg, 0.01-10 mg/kg, 0.01-20 mg/kg, 0.01-30 mg/kg, 0.01-40 mg/kg, 0.01-50 mg/kg, 0.01-60 mg/kg, 0.01-70 mg/kg, 0.01-80 mg/kg, 0.01-90 mg/kg, 0.01-100 mg/kg, 0.01-150 mg/kg, 0.01-200 mg/kg, 0.01-250 mg/kg, 0.01-300 mg/kg, 0.01-500 mg/kg, and all ranges and points in between. In alternative embodiments dosage ranges include, but are not limited to, 0.01-1 mg/kg, 1-10 mg/kg, 10-20 mg/kg, 20-30 mg/kg, 30-40 mg/kg, 40-50 mg/kg, 50-60 mg/kg, 60-70 mg/kg, 70-80 mg/kg, 80-90 mg/kg, 90-100 mg/kg, 100-150 mg/kg, 150-200 mg/kg, 200-300 mg/kg, 300-500 mg/kg, and all ranges and points in between.

The blood plasma concentration of proTAME or the composition containing proTAME can be about 0.1 μM to about 1000 μM, about 0.1 μM to about 1 μM; about 0.1 μM to about 10 μM; about 10 μM to about 100 μM; about 100 μM to about 500 μM, about 500 μM to about 1000 μM, and any micromolar concentration in between. Alternatively, or in addition, the cerebral spinal fluid concentration of proTAME or the composition containing proTAME can be about 0.1 μM to about 1000 μM, about 0.1 μM to about 1 μM; about 0.1 μM to about 10 μM; about 10 μM to about 100 μM; about 100 μM to about 500 μM, about 500 μM to about 1000 μM, or any micromolar concentration in between.

The pharmaceutical composition can be administered at a dosage from about 1 mg/m² to 5000 mg/m² per day, about 1 mg/m² to 10 mg/m² per day, about 10 mg/m² to 100 mg/m² per day, about 100 to 1000 mg/m² per day, about 1000 to 2500 mg/m² per day, about 2500 to 5000 mg/m² per day, or any daily mg/m² dosage in between. Preferably, 1 mg/m² to 5000 mg/m² per day is the administered dosage for a human.

Subjects of the invention either present a sign or symptom of a cell proliferative disease and/or have been diagnosed with a cell proliferative disease. A sign is an external and visible indication that something is not right in the body. Signs are signals that can be observed by family members, colleagues, and medical professionals. For instance, a fever, abnormal or labored breathing, or abnormal lung sounds heard through a stethoscope are examples of signs. A symptom is a different kind of signal of disease, illness, injury, or that something is not right in the body. Symptoms are only felt or noticed by the person who has them, but are not usually obvious to the outside observer. For example, fatigue, pain, and feeling short of breath are examples of symptoms.

Typical signs or symptoms include, but are not limited to, tumors (benign or malignant), pain (for instance, as a tumor grows and exerts pressure on surrounding vasculature and organs), fever, fatigue, weight loss (as the proliferating cells drain energy resources from the body), changes to immune system function (decreased immune function or autoimmune episodes), presence of secreted factors in the bloodstream (from a cell mass or the metasis thereof), weakness, dizziness, and blood clots (particularly in the legs), unexplained bleeding, the appearance of wounds or sores and, optionally, the inability of these wounds to heal, and visible changes to the skin (e.g., melanoma).

Preferably, subjects are human; however, subjects of all animal species are contemplated. In particular, preferred animal species include, worm (e.g., C. elegans), frog (e.g., Xenopus), mouse, rat, guinea pig, ferret, bird, feline/cat (domestic and wild), canine/dog (domestic and wild), livestock (cattle, goats, sheep, swine), horse, and non-human primates (e.g. macaque, marmoset, tamarin, spider monkey, owl monkey, vervet monkey, squirrel monkey, baboon as well as great apes (gorillas, chimpanzees, and orangutans)). Subjects encompass both males and females. Subjects are of any age, including, but not limited to, neonatal, infant, child, adult, and elderly populations. Subjects may receive other therapies prior to, concurrently with, or following administration of the compositions and formulations of the invention.

All references cited herein are hereby incorporated by reference.

### BRIEF DESCRIPTION OF THE DRAWINGS

FIG. 1A is a schematic representation of the structure of proTAME and proAAME.

FIG. 1B is a graph showing that proTAME induces mitotic arrest in HeLa cells. HeLa cells expressing H2B-GFP were treated with compounds and imaged for 36 h at 15 min intervals. Cumulative frequency curves of mitotic duration were plotted.

FIG. 1C is a graph showing that proAAME does not induce mitotic arrest in HeLa cell. The same experiment in 1B was done with proAAME.

FIG. 1D is a bar graph showing that cell fate distribution for the experiments in 4B and 4C.

FIG. 1E is a series of photographs of gel electrophoresis analysis showing that proTAME stabilizes endogenous APC substrates. HeLa cells were synchronized by double thymidine procedure and released into compounds (12 μM). Protein levels were measured by immunoblot.

FIG. 1F is a series of photographs and accompanying graphs showing that proTAME stabilizes exogenous cyclin B1-GFP and cyclin A2-GFP in HeLa cells. HeLa H2B-RFP cells transduced with cyclin-GFP adenoviruses were imaged at 12 min intervals after treatment with 20 μM proTAME or proAAME, or 150 nM nocodazole. Representative cells are shown. The fraction of GFP intensity remaining at 60 min as compared to the onset of mitosis was determined for at least 30 individual cells for each group. Error bars represent standard error of the mean.

FIG. 1G is a series of photographs and accompanying schematics showing that proTAME partially inhibits Cdc20 binding to human APC. HeLa cells were released from a double thymidine block into growth medium for 10 h, and then treated with 10 μM MG132 plus DMSO, proTAME or proAAME (12 μM) for an additional 2 h. APC was immunoprecipitated with Cdc27 antibody, and the amount of Cdc27 and Cdc20 was analyzed by immunoblot.

FIG. 1H is a series of photographs and accompanying schematics showing that proTAME inhibits APC-Cdc20 activity. APC from Hela cells treated as in 4G was tested in an in vitro ubiquitination assay.

FIG. 2A is a chromatogram and accompanying graph. 50 μM proTAME was added to interphase Xenopus extract and samples were collected at indicated time points. Ethyl acetate extraction was performed, and samples analyzed by liquid-chromatography-mass spectrometry (LC/MS). Chromatograms and quantitation of the abundance of TAME and proTAME ion are shown.

FIG. 2B is a graph showing that proTAME inhibits cyclin B-luciferase degradation in mitotic extract. Different concentrations of proTAME or proTAME were added to mitotic Xenopus extract containing cyclin B-luciferase reporter. Samples were collected at 60 min and the remaining reporter level was measured by luminescence.

FIG. 2C is a chromatogram and accompanying graph showing that proTAME is efficiently activated in HeLa cells but not MCF10A cells. HeLa and MCF10A cells were treated with 20 μM proTAME. Cells were collected at indicated time points and lysed. Ethyl acetate extraction was performed prior to LC/MS analysis. Chromatograms and quantitation of the abundance of TAME ion normalized to total protein level are shown. Notice that the scale in the top panels is different for HeLa cells (maximum value 140,000) and MCF10A cells (maximum value 14,000).

FIG. 3 is a series of photographs showing that proTAME induces mitotic arrest in synchronized HeLa cells. HeLa cells were synchronized with double thymidine block and released into 0.06% DMSO, 12 μM proTAME or 12 μM proTAME after the second block. Phase contrast images were taken every 4 h after release.

FIG. 4A is a series of photographs and accompanying graph showing that proTAME does not disrupt mitotic spindles. HeLa cells were treated with DMSO, proTAME (12 μM), Taxol™ or nocodazole (300 nM) for 2 h, and then stained with tubulin (green) and CREST (red) antibody. Representative images are shown. Bar: 3 mm. Representative images of kinetochore pairs are shown. Bar: 1.2 μm. Inter-kinetochore distance was measured in DMSO or proTAME treated cells and no statistically significant difference was found (n=55, P=0.3, paired t-test).

Error bars represent standard deviation.

FIG. 4B is a graph showing that proTAME-induced mitotic arrest is partially Mad2-dependent. Synchronized HeLa cells were transfected with Mad2/control siRNA followed by drug treatments. Cumulative frequency curves of mitotic duration were plotted.

FIG. 4C is a graph of the cell fate distribution of the experiments in 4B.

FIG. 4D is a series of photographs of gel electrophoresis analysis showing that proTAME-induced mitotic arrest is partially Mad2-dependent. HeLa cells were treated as in 4B. Protein levels were measured every 4 h by Western blot.

FIG. 4E is a series of photographs showing that proTAME rescues the mitotic defect induced by Mad2 knockdown. HeLa cells expressing H2B-GFP were treated with Mad2 siRNA 24 h prior to addition of DMSO or proTAME (12 μM). Cells were imaged every 3 minutes using a 40× objective.

FIG. 5A is a series of photographs showing that proTAME induced mitotic arrest is partially Mad2-dependent. HeLa cells were synchronized with double thymidine block and released into 0.06% DMSO, 12 μM proTAME, 300 nM nocodazole or 12 μM proTAME plus 300 nM nocodazole after the second block. Samples were collected every 4 h and protein level was measure by Western blot.

FIG. 5B is a series of photographs showing that proTAME induced mitotic arrest is partially Mad2-dependent. HeLa cells were treated as in FIG. 5A. Phase contrast images were taken every 4 h after release.

FIG. 6 is a series of photographs showing that Mad2 knockdown efficiently overrides the spindle assembly checkpoint in the presence of nocodazole. Asynchronous HeLa H2B-GFP cells were transfected with Mad2 siRNA 24 h prior to treatment with 300 nM nocodazole. Live imaging was done at 40× magnification and 3 min interval.

FIG. 7A is a graph showing the effects of Mad2 knockdown on proTAME-induced mitotic arrest determined by DIC imaging. Asynchronous HeLa cells were transfected with Mad2 or control siRNA. 24 h after transfection, the cells were treated with 0.06% DMSO, 12 μM proTAME, 300 nM nocodazole or 12 μM proTAME plus 300 nM nocodazole. DIC imaging was performed for 48 h at 15 min interval.

FIG. 7B is a graph of cumulative frequency and cell fate distribution based on the results of FIG. 7A. The Mitotic duration of each division was determined and cumulative frequency curves and cell fate distribution were plotted.

FIG. 8 is a showing that proteasome inhibitor-induced mitotic arrest is partially Mad2-dependent. HeLa cells were synchronized with double thymidine block, transfected with Mad2 or control siRNA after the first block, and released. 3 μM MG132 was added to Mad2 knockdown cells at 8.5 h post release and to control cells at 9.5 h post release because Mad2 knockdown cells enter mitosis faster than control cells. Live imaging was performed for 48 h at 15 min interval. Mitotic arrest of each division was determined as the time between anaphase and the addition of MG132 and cumulative frequency curves were plotted.

FIG. 9A is a graph showing that fluorescence imaging accelerates proTAME-induced cell death. HeLa H2B-GFP cells were treated with 12 μM proTAME or 0.06% DMSO. Live cell imaging was performed using differential interference contrast (DIC) optics for 48 h at 15 min intervals. For comparison, the curves obtained in H2B-GFP fluorescent imaging experiment shown in FIG. 1B were plotted on the same graph.

FIG. 9B is a graph of cumulative frequency and cell fate distribution based on the results of FIG. 9A. Mitotic duration of each division was determined and cumulative frequency curves and cell fate distributions were plotted.

FIG. 10A is a series of schematic diagrams depicting the structures of TAME and AAME.

FIGS. 10B-F are a series of immunoblots showing that TAME inhibits APC activation by perturbing binding of Cdc20 or Cdh1. (B) TAME induces mitotic arrest in Xenopus extract. Recombinant cyclin B1/Cdk1 was added to interphase extract in the presence of compounds. Cdc27 phosphorylation and cyclin B1 levels were examined by immunoblot. (C) TAME inhibits APC activation. Compounds were added to mitotic Xenopus extract immediately before APC immunoprecipitation. The activity of the isolated APC was measured in a reconstituted assay. (D) TAME inhibits Cdc20 association with mitotic APC. Compounds were added to mitotic Xenopus extract prior to APC immunoprecipitation. Numbers represent CCD-imaging based-intensity quantitation of the immunoblot, and show the relative amount of Cdc20 normalized to Cdc27. (E) TAME inhibits Cdh1 association with interphase APC. Interphase Xenopus extract was pre-incubated with compounds for 30 min prior to adding recombinant Cdh1 and APC immunoprecipitation. (F) TAME inhibits APC activation by Cdh1. Interphase Xenopus extract was pre-incubated with compound for 30 min prior to adding recombinant Cdh1 and APC immunoprecipitation. The activity of the isolated APC was measured in a reconstituted assay.

FIG. 11A is a graph showing that TAME stabilizes cyclin B-luciferase (cycB-luc) reporter in mitotic Xenopus extract. Different concentrations of TAME were added to the extract containing the reporter. Samples were collected at 60 min and the remaining reporter level was measured by luminescence. Interphase extract was used as a negative control.

FIG. 11B is a series of schematic diagrams depicting the structures of TAME derivatives.

FIG. 11C is a graph depicting the results of a luciferase assay. The derivatives shown in (A) were tested in the luciferase assay at 200 μM.

FIG. 11D is a graph showing that TAME does not inhibit degradation of pre-ubiquitinated cyclin B. Baculovirus-expressed and purified ³⁵S-labeled cyclin B bound to unlabeled Cdk1 was first ubiquitinated by APC in an in vitro ubiquitination system and then added into Xenopus extract supplemented with DMSO, 200 μM TAME or 200 μM proteasome inhibitor MG262. At indicated time points, protein was precipitated and the level of radioactivity in supernatant was measured by scintillation counting.

FIG. 11E is a photograph of an immunoprecipitation product analyzed using gel electrophoresis. The results demonstrate that TAME does not affect APC composition of APC core subunits. Mitotic extract was treated with DMSO or 200 μM TAME. APC was immunoprecipitated and the subunits were resolved by SDS-PAGE and visualized by coomassie stain. Identity of subunits was confirmed by mass spectrometry.

FIG. 12A is a paired graph and immunoblot showing that TAME binds to the APC and inhibits binding of the IR tail of activator proteins. TAME binds Xenopus APC. ³H-TAME was added to interphase extract or to extract that had been partially or completely immunodepleted of APC. Remaining APC was then immunoprecipitated and the associated radioactivity was measured by scintillation counting. Residual APC levels were measured by immunoblot with Cdc27 antibody. Specific binding was calculated as described in the methods.

FIG. 12B is a graph showing that unlabeled TAME competes with ³H-TAME for binding to Xenopus APC. ³H-TAME was added to interphase extract with unlabeled TAME or AAME prior to APC immunoprecipitation.

FIG. 12C is a paired graph and immunoblot showing that ³H-TAME binds to human APC. The experiment in FIG. 12A was repeated with lysate from asynchronous HeLa cells.

FIG. 12D is a series of schematic diagrams of Cdc20, the C-box containing fragment, and structures of the IR tail and TAME.

FIG. 12E is a pair of immunoblots showing that TAME inhibits the interaction between the Cdh1 C-terminal IR peptide and the APC. Left: Resin coupled with cysteine (Ctrl resin), Cdh1 C-terminal peptide (WT), or the peptide lacking the C-terminal isoleucine and arginine (ΔIR) was incubated with interphase Xenopus extract, washed, and the amount of bound Cdc27 was analyzed by immunoblot. Right: Cdh1 C-terminal resin was incubated with interphase extract in the presence of compounds and the amount of Cdc27 was analyzed as above.

FIG. 12F is an immunoblot showing that TAME does not inhibit the interaction between the C-box and the APC. A 159-amino acid N-terminal fragment of Cdc20 containing the C-box fused to GST (GST-CDC20 N159 WT) or the same fragment lacking the C-box (GST-CDC20 N159 AC-box) were bound to glutathione resin and incubated with mitotic Xenopus extract in the presence of compounds. Bound Cdc27 was analyzed by immunoblot.

FIG. 12G is an immunoblot showing that TAME inhibits IR-peptide crosslinking to APC subunits. Purified interphase Xenopus APC was incubated with an IR peptide coupled to a biotin-containing label-transfer reagent, in the presence or absence of compounds, prior to photocrosslinking Reaction products were detected by streptavidin-HRP.

FIG. 13A is a graph depicting the ability of TAME derivates to compete with ³H-TAME for binding to the APC correlates with their ability to inhibit cyclin B-luciferase degradation. Two hundred nM ³H-TAME was added into interphase extract with 10 μM of unlabeled TAME derivatives before Cdc27 immunoprecipitation, and the amount of bound radioactivity was determined by scintillation counting. Specific binding was obtained by subtracting the value of mock IP (IP without Cdc27 antibody). The relative levels of competition correlate with the trends seen in the cyclin-luciferase assay as shown in FIG. 11C.

FIG. 13B is an immunoblot showing that Cdh1 C-terminal peptide crosslinks to the TPR subcomplex in an IR-dependent manner. An IR peptide coupled to a photocrosslinker labels a subset of APC subunits. Left: Coomassie stain of APC immunopurified from interphase Xenopus extract. Right: APC subunits crosslinked by the labeled IR peptide. Identity of APC subunits was confirmed by mass spectrometry.

FIG. 13C is an immunoblot showing that crosslinking is IR-dependent. The crosslinking assay was performed in the presence of excess unlabeled IR peptide with the N-terminal cysteine blocked with N-ethyl maleimide (NEM) (lane 2), no UV illumination (lane 3) or with labeled ΔIR peptide (lane 4).

FIG. 14 is an immunoblot showing that TAME inhibits binding of wild type Cdc20 to the APC, but not binding of a ΔIR mutant. Mitotic APC immunoprecipitated from Xenopus extract was washed with XB high salt (500 mM KCl) and XB to remove endogenous Cdc20 prior to incubation with in vitro translated wild type Xenopus Cdc20 or the ΔIR mutant. Various competitors were added during incubation as indicated. Unbound proteins were washed away and bound Cdc20 was analyzed by immunoblot. Numbers represent the amount of Cdc20 normalized to Cdc27.

FIG. 15A is a series of schematic diagrams depicting structures of proTAME and proAAME.

FIG. 15B is a graph showing that proTAME inhibits APC activity in Xenopus extract and inhibits Cdh1-dependent APC activity during interphase in HeLa cells. ProTAME inhibits cyclin B-luciferase degradation in mitotic Xenopus extract. Different concentrations of proTAME or proAAME were added to mitotic Xenopus extract containing cyclin B-luciferase reporter. Samples were collected at 60 min and the remaining reporter level was measured by luminescence.

FIG. 15C is a series of immunoblots showing that proTAME blocks Cdh1 association with the APC. HeLa cells were released from nocodazole and treated with proTAME in G1. APC was immunoprecipitated from cell lysates and the amount of Cdc27 and Cdh1 was analyzed by immunoblot.

FIG. 15D is a graph showing that proTAME restores mitotic entry in Emi1-depleted cells. HeLa cells were transfected with control siRNA or Emi1 siRNA and treated with DMSO or proTAME 24 h after transfection and then imaged for 48 h. About 400 cells were analyzed in each experiment, and the proportion that failed to enter mitosis during the 48 h of imaging was calculated. Results of 3 independent experiments are shown. Statistical significance was calculated using an unpaired t-test.

FIG. 15E is a graph showing that proTAME causes a mitotic entry delay if added during S-phase. HeLa H2B-GFP cells were released from a double thymidine block and proTAME (12 μM) was added at different time points as indicated. Mitotic entry was monitored by time-lapse imaging. Cumulative frequency curves of the time of mitotic entry are shown. Statistical analysis, including mean, median, statistical significance and number of cells analyzed per condition for all experiments is included in Table 1.

FIG. 16A is a series of graphs depicting the results of liquid-chromatography-mass spectrometry (LC/MS) analysis. ProTAME is converted to TAME in Xenopus extract. Fifty μM proTAME was added to interphase Xenopus extract and samples were collected at indicated time points. Ethyl acetate extraction was performed, and samples analyzed by liquid-chromatography-mass spectrometry (LC/MS). Chromatograms of the abundance of TAME and proTAME ion are shown.

FIG. 16B is a graph showing that ProTAME is efficiently activated in HeLa cells but not MCF10A cells. HeLa and MCF10A cells were treated with 20 μM proTAME. Cells were collected after 1 h and lysed. Ethyl acetate extraction was performed prior to LC/MS analysis. Quantitation of the abundance of TAME ion normalized to total protein level is shown.

FIG. 16C is a series of immunoblots showing that proTAME does not induce premature accumulation of APC substrates in G1 cells. HeLa cells were synchronized by double thymidine block, released into nocodazole for 13 h and washed out of nocodazole for 6 h. Cells were then treated with DMSO (0.06%), proTAME or proAAME (12 μM). Samples were collected at indicated time points and protein level was measured by immunoblot.

FIG. 17A is a schematic diagram depicting the experimental timeline and a graph showing that proTAME induces mitotic arrest in HeLa cells. Double thymidine synchronized HeLa H2B-GFP cells were treated with compounds and analyzed by time-lapse imaging. Cumulative frequency curves of mitotic duration and cell fate distributions are shown.

FIG. 17B is a schematic diagram depicting the experimental timeline and a graph showing that proTAME induces mitotic arrest without disrupting the mitotic spindle. Partial Cdc20 knockdown sensitizes cells to proTAME treatment. Asynchronous HeLa H2B-GFP cells were transfected with control or Cdc20 siRNA 24 h prior to treatment with compounds.

FIG. 17C is a series of immunoblots showing that proTAME stabilizes endogenous APC substrates. Double thymidine synchronized HeLa cells were treated with compounds.

FIG. 17D is a series of photographs of immunohistochemistry and accompanying graphs showing that proTAME stabilizes exogenous cyclin B1-GFP and cyclin A2-GFP in HeLa cells. HeLa H2B-RFP cells transduced with cyclin-GFP adenoviruses were treated with 20 μM proTAME or proAAME, or 150 nM nocodazole. Representative cells are shown. For quantitation, the fraction of GFP intensity remaining at 60 min as compared to the onset of mitosis was determined (n≧30 individual cells per treatment). Error bars represent standard error of the mean.

FIG. 17E is a series of photographs of immunohistochemistry and an accompanying graph showing that proTAME does not disrupt mitotic spindles or alter interkinetochore distance. Asynchronous HeLa cells were treated with compounds for 2 h, and then stained with anti-tubulin (green) and CREST (red) antibody. Representative images are shown. Bar: 3 μm. Representative images of kinetochore pairs are shown. Bar: 1.2 μm. Inter-kinetochore distance was measured in DMSO or proTAME treated cells (n=55, P=0.23). Error bars represent standard deviation.

FIG. 18A is a graph and table showing that proTAME induces a mitotic delay in hTERT-RPE1 H2B-GFP cells. Asynchronous hTERT-RPE1 H2B-GFP cells were treated with 0.06% DMSO, 6 μM proTAME or 12 μM proAAME. Cells were imaged at 12 min interval. Cumulative frequency curves of mitotic duration and cell fate distribution are shown.

FIG. 18B is a series of immunoblots showing that partial Cdc20 knockdown sensitizes HeLa H2B-GFP cells to proTAME treatment. HeLa H2B-GFP cells were plated in 24-well plates and transfected with indicated siRNAs. Control siRNA#3 was added to final 18.5 nM siRNA concentration in wells with decreasing Cdc20 siRNA concentration. Cells were lysed 48 h after transfection and lysates were subjected to western blotting to detect Cdc20 and GAPDH. Protein level quantification was performed on a LiCor Odyssey scanner as described in Material and Methods.

FIG. 18C is a graph showing that a high concentration of proTAME induces a mild delay in chromosome congression. HeLa H2B-GFP cells were synchronized by double thymidine block and treated with 0.06% DMSO, 3 μM proTAME, 12 μM proTAME, 10 μM MG132 or 10 nM nocodazole at 8 h after release. Cells were imaged at 3 min interval and 40× magnification. The time between prophase and full metaphase congression was analyzed. Cumulative frequency curves of the congression time were plotted.

FIG. 19A is a schematic diagram depicting the experimental timeline and a graph showing that proTAME-induced mitotic arrest is SAC-dependent. ProTAME-induced mitotic arrest is Mad2-dependent. HeLa H2B-GFP cells were transfected with indicated siRNAs between rounds of thymidine treatment. Following release, cells were treated with compounds and analyzed by time-lapse imaging. A graph of the same data with an expanded x-axis is shown in FIG. 20A.

FIG. 19B is a series of photographs showing that proTAME rescues the mitotic defect induced by Mad2 knockdown. Asynchronous HeLa H2B-GFP cells were treated with Mad2 siRNA 24 h prior to addition of compound.

FIG. 19C is a schematic diagram depicting the experimental timeline and a graph showing that proTAME-induced mitotic arrest is hesperadin-sensitive. Double thymidine synchronized HeLa H2B-GFP cells were treated with compounds 8 h following release.

FIG. 19D is a schematic diagram depicting the experimental timeline and a graph showing that UbcH10 or Cdc27 knockdown induces a hesperadin-sensitive mitotic delay. HeLa H2B-GFP cells were transfected with indicated siRNA between rounds of thymidine synchronization and treated with hesperadin 8 h following release.

FIG. 20A is a graph showing the same experiment as shown in FIG. 19A but with an expanded x-axis to better show the difference between short mitotic durations.

FIG. 20B is a series of immunoblots from the experiment shown in FIG. 19A.

FIG. 20C is a series of photographs showing that Mad2 knockdown efficiently overrides the spindle assembly checkpoint in the presence of nocodazole. Above: Control-siRNA-treated cells were treated with 0.06% DMSO. Below: Mad2 knockdown cells were treated with 300 nM nocodazole. Live imaging was done at 40× magnification and 3 min interval.

FIG. 20D is a series of immunoblots showing that proTAME inhibits Cdc20 association with human APC if the SAC is inactivated. Double thymidine synchronized HeLa cells were transfected with Mad2 and BubR1 siRNA, and infected with cyclin BΔ107 adenovirus. At 10.5 h after release from thymidine, cells were treated with indicated drugs for 2 h. M/B: Mad2/BubR1 siRNA. Numbers indicate the relative Cdc20 band intensity normalized to Cdc27.

FIG. 21A is a series of immunoblots showing that hesperadin overrides a proTAME-induced mitotic arrest. HeLa cells were synchronized with double thymidine block and treated with indicated drugs at 10 h after release for 1 h. Mitotic cells were collected and APC was immunoprecipitated from cell lysate. Protein levels were measured by Western blot.

FIG. 21B is a series of photographs showing that hesperadin induces deformation of the metaphase plate in proTAME-arrested cells. HeLa H2B-GFP cells were synchronized with double thymidine block and treated with 12 μM proTAME at 8 h after release and with 100 nM hesperadin at 10 h after release. For cells that have entered metaphase prior to hesperadin treatment, changes in the morphology of metaphase plate were analyzed and one representative cell is shown. The yellow arrow denotes the time of hesperadin addition. A representative cell arrested with proTAME alone is also shown for comparison.

FIG. 21C is a graph, table, and accompanying immunoblot showing that hesperadin overrides proTAME-induced mitotic arrest of cells released from nocodazole-induced arrest. HeLa H2B-GFP cells were synchronized with double thymidine block and treated with 300 nM nocodazole at 8 h after release from thymidine block. Cells were washed out of nocodazole at 15 h after release from thymidine block into growth medium, or 12 μM proTAME, or 12 μM proTAME and 100 nM hesperadin. Cells were imaged at 12 min interval. Cumulative frequency curves of mitotic duration and cell fate distribution are shown. Cell samples were collected at different time points as indicated and protein levels were measured by immunoblot. The top diagram shows timing of treatments. N: nocodazole.

FIG. 22A is a schematic diagram depicting the experimental timeline, graph, and accompanying table showing that MG132-induced mitotic arrest is SAC-dependent. MG132-induced arrest is Mad2-dependent. HeLa H2B-GFP cells were transfected with indicated siRNAs between rounds of thymidine synchronization, treated with compounds, and followed by time-lapse imaging.

FIG. 22B is a schematic diagram depicting the experimental timeline, graph, and accompanying table showing that MG132-induced arrest is hesperadin-sensitive, but mitotic exit can be suppressed by proTAME. Double thymidine synchronized HeLa cells were treated with compounds.

FIG. 22C is a schematic diagram depicting the experimental timeline, graph, and accompanying table showing that Taxol™ cannot restore mitotic arrest in the presence of MG132 and hesperadin. Double thymidine synchronized HeLa cells were treated with compounds.

FIG. 22D is a schematic diagram depicting the experimental timeline, graph, and accompanying table showing that mitotic arrest induced by a higher concentration of MG132 remains hesperadin-sensitive. Double thymidine synchronized HeLa cells were treated with compounds. M: MG132; pT: proTAME; H: hesperadin.

FIG. 23A is a series of graphs showing that hepseradin overrides MG132-induced mitotic arrest. HeLa H2B-GFP cells were synchronized with double thymidine block and treated with indicated drugs at 10 h after release. Mitotic duration of each individual cell is plotted against its mitotic entry time point. The red line denotes the time of addition of MG132.

FIG. 23B is a series of immunoblots showing that HeLa cells were synchronized with double thymidine block and treated with indicated drugs at 10 h after release for 2 h. Mitotic cells were collected and APC was immunoprecipitated from cell lysate. Protein levels were measured by immunoblot.

FIG. 23C is a series of photographs showing that hesperadin induces deformation of the metaphase plate in MG132-arrested cells. HeLa H2B-GFP cells were synchronized with double thymidine block and treated with 10 μM proTAME at 10 h after release, and with 100 nM hesperadin at 11 h after release. For cells that have entered metaphase prior to hesperadin treatment, changes in the morphology of metaphase plate after hesperadin treatment were analyzed and one representative cell is shown. The yellow arrow denotes the time of hesperadin addition. A representative cell arrested with MG132 alone is also shown for comparison.

FIG. 24A is a schematic diagram depicting the experimental timeline, graph, and accompanying table showing that microtubule inhibitors require protein synthesis for mitotic arrest whereas proTAME and MG132 do not. Mitotic arrest induced by microtubule inhibitors requires protein synthesis but proTAME-induced arrest does not. Double thymidine synchronized HeLa-H2B-GFP cells were treated with compounds and followed by time-lapse imaging. CHX: cycloheximide.

FIG. 24B is a schematic diagram depicting the experimental timeline, graph, and accompanying table showing that MG132 (10 μM)-induced arrest is cycloheximide-resistant but Mad2-dependent. HeLa cells were transfected with indicated siRNAs between rounds of thymidine synchronization.

FIG. 24C is a schematic diagram depicting the experimental timeline, graph, and accompanying table showing that MG132 (10 μM)-induced arrest is cycloheximide-resistant but hesperadin-sensitive. Double thymidine synchronized HeLa cells were treated with compounds.

FIG. 24D is a schematic diagram depicting a model summarizing the mutual antagonism between the SAC and the APC. In the bottom panels, the x-axis indicates time from mitotic entry.

FIG. 25 is a series of immunoblots showing proteins required for maintenance of the SAC are synthesized during prolonged mitotic arrest. HeLa H2B-GFP cells were arrested in interphase by a 24-hour thymidine block (2 mM) and released in growth medium. To identify newly translated proteins in interphase cells, cells were switched to methionine-free labeling medium 3 hrs after release and de novo translated proteins were labeled by adding the methionine analog L-azidohomoalanine (AHA) at 250 μM for 3 hrs and collected by trypsinization. For labeling of proteins newly translated during mitosis, cells were released from thymidine block and nocodazole (300 nM) or proTAME (12 μM) was added 5 hrs after release. Seven hours later, mitotic cells were collected by shakeoff and switched to the labeling medium and incubated with 250 μM AHA for 12 hours. A majority of cells remained in mitosis based on morphology. Following the labeling period, mitotic cells were collected by shake off. A labeling reaction including cycloheximide 25 μg/mL as negative control was carried in parallel for each condition. Cells in nocodazole+cycloheximide slipped out of mitosis after the 12 hrs incubation and were collected by trypsinization. Protein lysates were generated for each labeling condition, and newly synthesized proteins were labeled using biotin azide and purified with neutravidin-agarose resin as described in the Methods section. Purified proteins from equivalent amounts of total protein were eluted by boiling in SDS sample buffer, separated on SDS-PAGE gels and indicated proteins were detected by western blotting.

FIG. 26 is a pair of graphs depicting the percentage of cells that have exited the cell cycle versus the mitotic duration for cells treated with either control (top trace), Taxol, proTAME, or a combination of Taxol and proTAME (bottom trace). The table correlates to the top graph. The bottom graph depicts the mitotic fate of cells treated to each condition.

FIG. 27 is a pair of graphs depicting the percentage of cells that have exited the cell cycle versus the mitotic duration for cells that are untreated (top trace), or alternatively, treated with proTAME, MG132, or a combination of MG132 and proTAME, each at varying concentrations. The table correlates to the top graph. The bottom graph depicts the mitotic fate of cells treated to each condition.

### DETAILED DESCRIPTION

The invention provides new insights into APC regulation and indicates that the APC is a druggable target amenable to pharmacologic intervention. Most cells that are mitotically arrested following proTAME treatment undergo cell death. Lower doses of proTAME induce mitotic delay rather than cell death, suggesting that APC inhibitors may be useful for restoring normal chromosome segregation in cases where spindle checkpoint function is compromised. For example, APC inhibitors may be useful for rescuing mitotic failure in preimplantation embryos generated through in vitro fertilization. Given that APC also regulates functions in the central nervous system and controls rates of axon growth, inhibition of Cdh1 association with the APC may also be of therapeutic benefit in the treatment of neurological disease.

The Anaphase-Promoting Complex/Cyclosome (APC) is a multi-subunit ubiquitin E3 ligase that promotes anaphase onset and mitotic exit by ubiquitinating cyclin B and securin to target them for proteolysis by the 26S proteasome (Pines, J. Nat Rev Mol Cell Biol 12, 427-438 (2011)). APC activity requires an activator. In mitosis, Cdc20 is the activator while in interphase, this role is taken by a homologous protein Cdh1 (Pesin, J. A. & On-Weaver, T. L. Annu Rev Cell Dev Biol 24, 475-499 (2008)). The activator contains a C-terminal seven WD40-repeats domain that has been shown to interact with a motif known as the Destruction box (D-box), an APC-specific degron on APC substrates (Kraft, C. et al. Mol Cell 18, 543-553 (2005)). The APC recognizes the D-box through a co-receptor formed by the activator and another core APC subunit Apc10 (Buschhorn, B. A., et al. Nat Struct Mol Biol 18, 6-13 (2011); da Fonseca, P. C., et al. Nature 470, 274-278 (2011)). Although additional APC-specific degrons such as the KEN box exist, and might be important for APC activity in late mitosis and interphase (Nguyen, H. G., et al. Mol Cell Biol 25, 4977-4992 (2005); Pfleger, C. M. & Kirschner, M. W. Genes Dev 14, 655-665 (2000)), the D-box is essential for APC Cdc20-driven degradation of cyclin B and securin prior to anaphase onset (Hagting, A., et al. J Cell Biol 157, 1125-1137 (2002); Clute, P. & Pines, J. Nat Cell Biol 1, 82-87 (1999)).

In prometaphase, the APC is activated by Cdc20, leading to ubiquitination and degradation of Nek2A and cyclin A. However, ubiquitination of other APC substrates is inhibited by the Spindle Assembly Checkpoint (SAC) until chromosomes have achieved proper bipolar attachment to the mitotic spindle. Once the SAC is satisfied, ubiquitination and degradation of securin and cyclin B lead to chromosome segregation and mitotic exit. In telophase, another APC activator, Cdh1, replaces Cdc20 and maintains APC activity during G1.

Cdc20 and Cdh1 are important for recruiting substrates to the APC. The activator proteins share several evolutionarily conserved motifs, including an N-terminal C-box (comprising the consensus sequence DRFYIPXR (SEQ ID NO: 1)), seven WD40 repeats (also known as WD or beta-transducin repeats of about 40 amino acids, often terminating in a WD dipeptide and containing 4-16 repeating units that together form a circular beta-propellar structure), and a C-terminal IR tail (a C-terminal region including one or more IR dipeptide motifs). Whereas the WD40 domain may interact simultaneously with substrates and the APC, the C-box and the IR tail are specifically involved in APC binding. The IR tail of Cdh1 interacts with multiple APC subunits, including Cdc27 and Apc7. Deletion of the IR tail of Cdh1 compromises its ability to activate human APC in vitro, and is lethal in budding yeast lacking Sic1. However, deletion of IR tail of Cdc20 does not affect the viability of wild-type budding yeast, and thus, does not seem to be strictly required for APC activation. Instead, the IR tail may be important for regulating Cdc20 abundance, as Cdc20ΔIR accumulates to higher levels than the wild-type protein. Thus while the IR tail seems to be critical for Cdh1 recruitment and activation of the APC, the specific role of IR tail of Cdc20 in APC binding and activation remains unclear.

The Spindle Assembly Checkpoint (SAC) ensures that, the replicated sister chromatids faithfully segregate into the two daughter cells during mitosis (Musacchio, A. & Salmon, E. D. Nat Rev Mol Cell Biol 8, 379-393 (2007)). During prometaphase, the kinetochores of each sister chromatid are attached to microtubule fibers emanating from one of the two opposite spindle poles, a configuration known as bi-orientation. Bi-oriented chromosomes then become aligned on an equator plane of the cell in a process named congression and the completion of congression is the hallmark of metaphase. The SAC senses unattached or improperly attached kinetochores and restrains the APC from ubiquitinating securin and cyclin B1 before metaphase is achieved. The effector of SAC-mediated APC inhibition is a complex known as the Mitotic Checkpoint Complex (MCC) that consists of stoichiometric amounts of Mad2, Cdc20, BubR1 and Bub3 (Sudakin, V. et al. J Cell Biol 154, 925-936 (2001)). Although Cdc20 as a component of the MCC can still bind to the APC (Herzog, F., et al. Science 323, 1477-1481 (2009); Zeng, X., et al. Cancer Cell 18, 382-395 (2010)), it is not capable of promoting APC substrate ubiquitination but instead seems to be susceptible to auto-ubiquitination (Reddy, S. K., et al. Nature 446, 921-925 (2007); Stegmeier, F., et al. Nature 446, 876-881 (2007)). The mechanism of MCC-mediated APC inhibition remains an unanswered question in cell biology. The MCC may alter the way that Cdc20 binds to the APC (Herzog, F., et al. Science 323, 1477-1481 (2009)). Moreover, it has been proposed that budding yeast Mad3 may act as a pseudo-substrate since it contains several APC degrons, including two N-terminal KEN-boxes and one C-terminal D-box, and competes with a canonical substrate for Cdc20 binding (Burton, J. L. & Solomon, M. J. Genes Dev 21, 655-667 (2007)). Interestingly, the roles of these degrons in Mad3 do not seem to be equivalent as KEN-box mutations fully abrogate Mad3 function but the D-box mutation is better tolerated (Burton, J. L. & Solomon, M. J. Genes Dev 21, 655-667 (2007)). The importance of the KEN-box is also confirmed in Drosophila, mouse and human cells but the role of the BubR1D-box has not been evaluated in higher organisms (Elowe, S., et al. J Cell Sci 123, 84-94 (2010); Malureanu, L. A., et al. Dev Cell 16, 118-131 (2009); Rahmani, Z., et al. J Cell Biol 187, 597-605 (2009)).

Microtubule inhibitors are used as anti-mitotic chemotherapy drugs for cancer treatment (Montero, A., et al. Lancet Oncol 6, 229-239 (2005)). They disrupt microtubule functions and activate the SAC to induce mitotic arrest in cells. Direct APC inhibition equally induces mitotic arrest without side effects associated with microtubule inhibition and therefore represents a better route for anti-mitotic drug development (Zeng, X., et al. Cancer Cell 18, 382-395 (2010); Montero, A., et al. Lancet Oncol 6, 229-239 (2005)). To identify potential small molecule APC inhibitors, a high throughput chemical screen was conducted to search for compounds that can stabilize a cyclin B1-luciferase reporter in mitotic Xenopus egg extract (Verma, R., et al. Science 306, 117-120 (2004). TAME was among the most potent hits from the screen. TAME acts as a structural analog of the IR tail of Cdc20 and competes for the same binding site on the APC (Zeng, X., et al. Cancer Cell 18, 382-395 (2010)). In the absence of APC substrates such as cyclin B1, TAME strongly inhibits the binding of free Cdc20 to the APC and induces auto-ubiquitination followed by dissociation of Cdc20 pre-bound to the APC. However, APC substrates promote free Cdc20 binding to the APC in the presence of TAME, presumably because forming the D-box co-receptor with Apc10 provides one addition contact point between APC and Cdc20 to render the IR-dependent interaction non-essential. A cell-permeable TAME prodrug (proTAME) induces a prolonged mitotic arrest in cells.

### EXAMPLES

### Example 1

### A TAME Prodrug Induces Mitotic Arrest in HeLa Cells by Inhibiting APC Activation

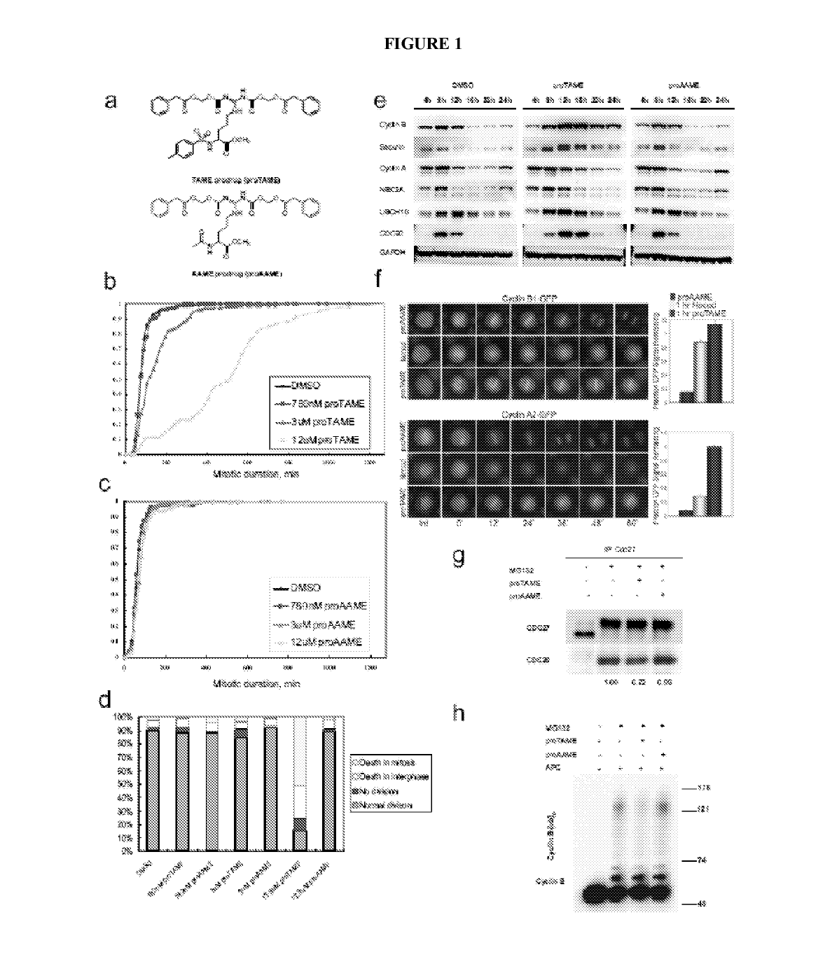

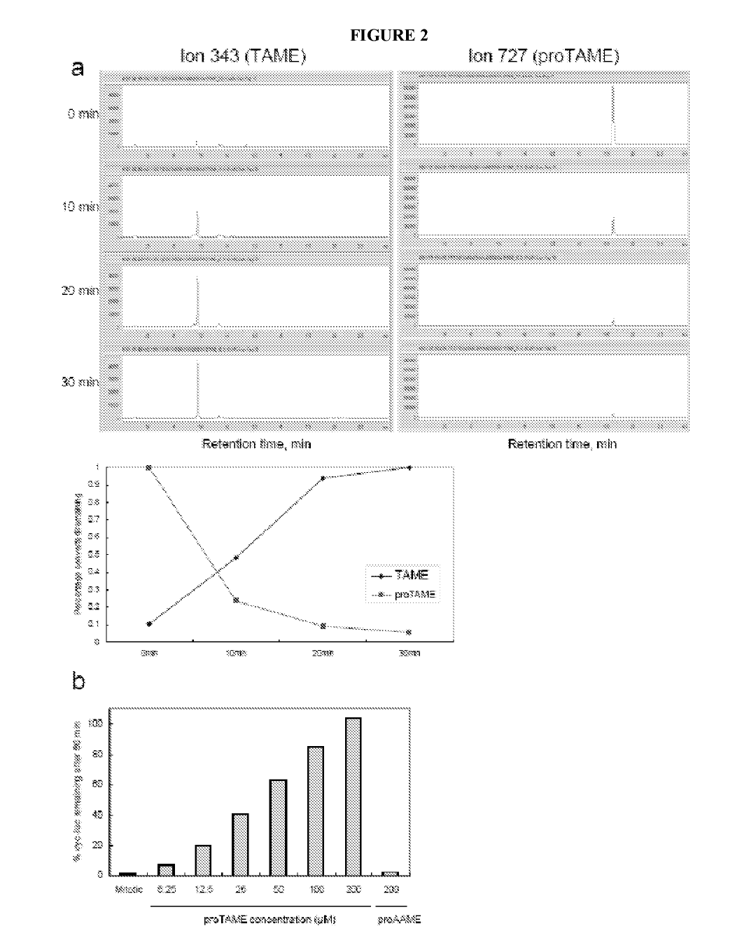

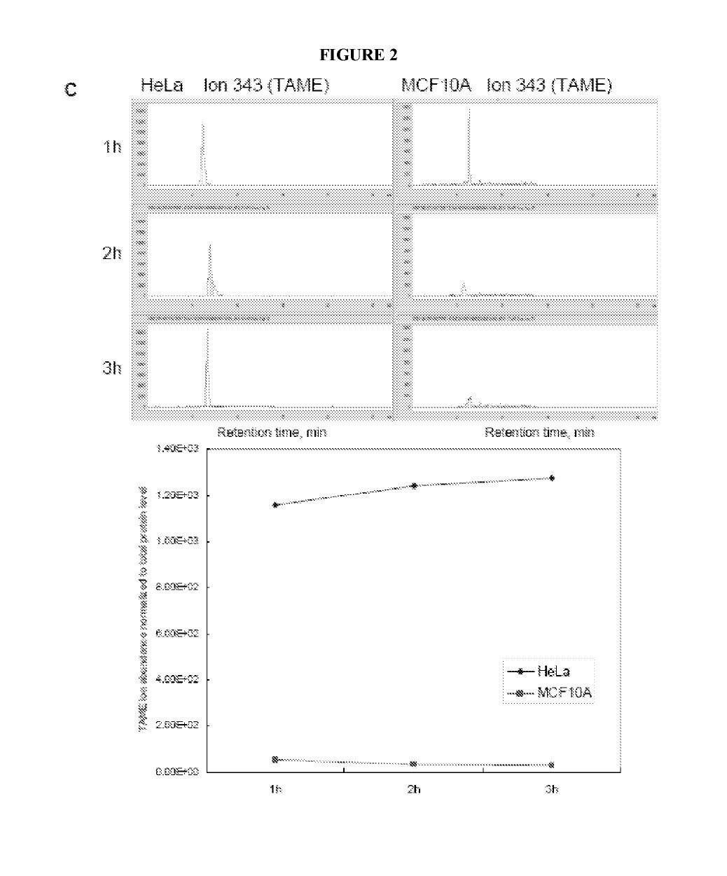

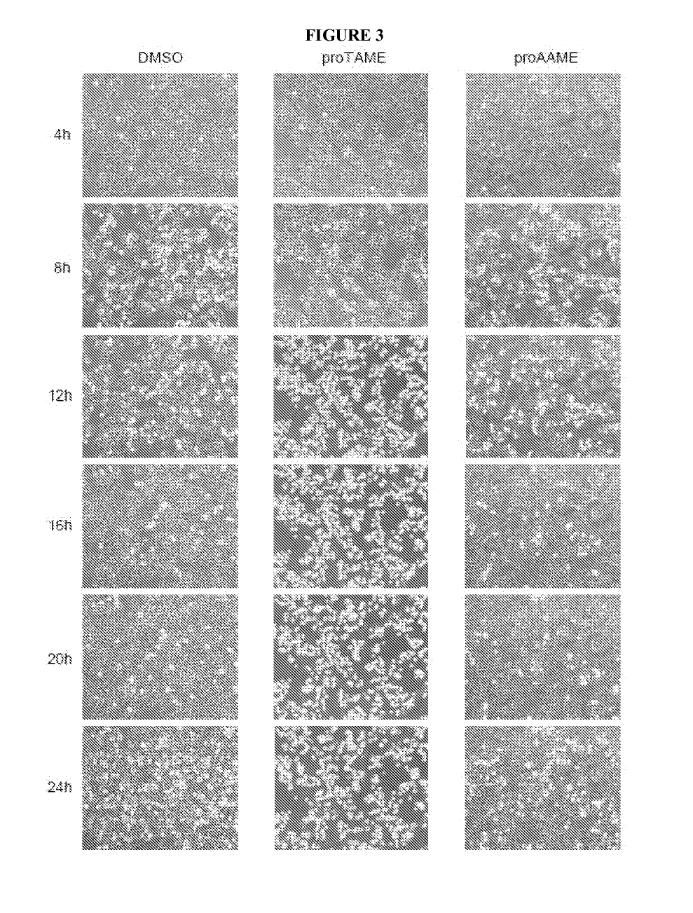

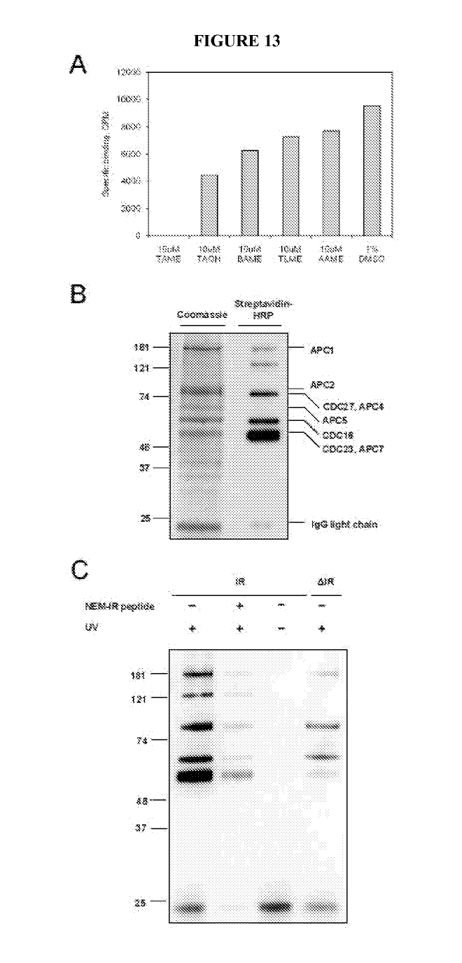

Despite the fact that TAME binds to human APC, it did not inhibit mitotic division in human HeLa cells. It was speculated that TAME might not be cell permeable due to its positively charged guanidino group. Therefore a cell-permeable TAME prodrug was synthesized by modification of the guanidino group (Saulnier et al., Bioorgan. & Med. Chem. Let., 1994, 4:1985-1990; hereby incorporated by reference) to produce an N,N′-bis(acyloxymethyl carbamate) derivative 18 (proTAME) (FIG. 1A). Once inside the cell, proTAME is cleaved by cellular esterases, producing an unstable carbamate intermediate that undergoes decarboxylation to restore the original structure of TAME. It was found that proTAME is rapidly converted to TAME in Xenopus extract (FIG. 13A) and efficiently inhibits cyclin B-luciferase proteolysis (FIG. 13B). ProTAME is also activated efficiently when added to HeLa cells, but conversion was not efficient in all cell lines tested (FIG. 13C).

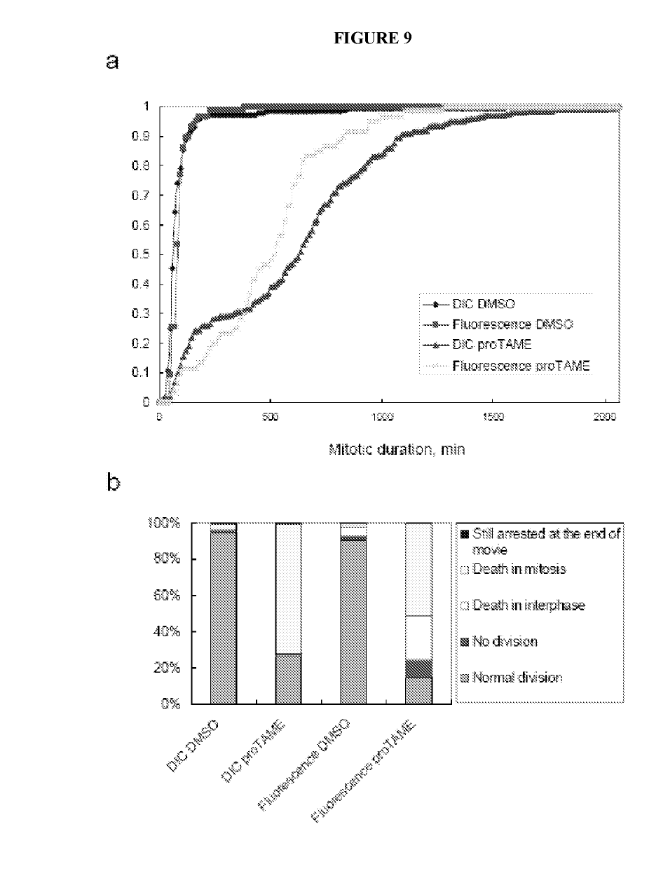

The effect of ProTAME on cell division was next determined by live imaging of HeLa cells that express Histone 2B-GFP. As a control, a prodrug version of AAME (proTAME) was synthesized (FIG. 1A). ProTAME induced a dose-dependent increase in mitotic duration, from 75 minutes for DMSO or proTAME treated cells, to 120 minutes for cells treated with 3 μM proTAME (FIGS. 1B and C). At this concentration, proTAME-treated cells formed metaphase plates with normal timing but anaphase onset was delayed. At 12 μM, proTAME-treated cells arrested in metaphase for more than 8 hours, with most cells dying after the prolonged arrest. At this concentration, chromosome congression was also delayed, with cells taking 40 minutes, on average, to achieve metaphase, compared to 15 minutes for DMSO-treated cells. The delay is potentially a consequence of cyclin A stabilization. Some cells showed abnormal segregation of chromosomes after prolonged arrest, suggesting incomplete degradation of securing. At 12 μM, proTAME had no effect on mitosis or cell viability (FIG. 1C). It was noted that fluorescence imaging may artifactually shorten the duration of proTAME-induced mitotic arrest by enhancing cell death, as repeating this experiment by Differential Interference Contrast (DIC) imaging rather than fluorescence imaging increased the duration of mitotic arrest from 8 to over 10 hours (FIGS. 9A, B). Under these imaging conditions, late dividing cells (those entering mitosis 16 hours after the initiation of the experiment) did not arrest in mitosis, but rather showed only a modest mitotic delay. Whether this difference reflects proTAME metabolism or whether exposure of cells to proTAME during G1 induces drug resistance is next determined.

The effect of proTAME on the degradation of endogenous APC substrates in synchronized HeLa cells was next determined. Cyclin B and securin were dramatically stabilized by proTAME but not by proTAME (FIG. 1E). Cyclin A and Nek2A, which are both degraded in early mitosis, were also stabilized but to a lesser extent. The effects of proTAME on degradation of cyclinB1-GFP or cyclin A2-GFP were also characterized by live cell imaging. In the absence of drug, both proteins were degraded during mitosis, with cyclin A2-GFP degradation preceding that of cyclin B1-GFP. As expected, cyclin B1-GFP was stabilized during nocodazole-induced mitotic arrest, whereas cyclin A2-GFP was degraded, since its degradation is not constrained by the SAC 3 (FIG. 1F). Consistent with the Western blot results, cyclin B1-GFP was stabilized in mitotic cells following proTAME treatment, and in fact accumulated to higher levels than in nocodazole-treated cells. Furthermore, cyclinA2-GFP was robustly stabilized during proTAME-induced mitotic arrest, strongly suggesting that TAME directly inhibits APC activity, and does not stabilize APC substrates simply by activating the SAC.

The ability of proTAME to stabilize cyclin A and other APC substrates suggested that proTAME inhibits APC activation in human cells. To test whether this effect is a consequence of inhibiting Cdc20 binding to the APC, HeLa cells were released from a double thymidine block for 10 hours, and then treated with MG132 plus proTAME or proTAME for 2 hours. Mitotic cells were collected by shakeoff, lysed, and the APC was isolated by immunoprecipitation. A 30% reduction was observed in Cdc20 binding to the APC after proTAME treatment (FIG. 1G). To test if this modest reduction of APC-bound Cdc20 resulted in inhibition of APC activity, an in vitro ubiquitination assay was performed with APC isolated as described above and it was found that proTAME treatment indeed inhibited APC activity, especially the ability of APC to catalyze formation of higher molecular weight ubiquitin conjugates (FIG. 1H).

ProTAME may induce a sustained mitotic arrest in HeLa cells despite the incomplete blockade of Cdc20 association. In addition to Cdc20, Apc10/Doc1 also has an IR tail, and Doc1 has been implicated in substrate recognition and processivity of the APC. However, it is not yet clear whether the IR tail of Doc1 is required for APC association or its ability to activate the APC. Alternatively, proTAME may interfere with substrate recruitment, such as the interaction of the MR-tail of Nek2A with the APC.

### Example 2

### ProTAME-Induced Mitotic Arrest is Partially Dependent on SAC Activity

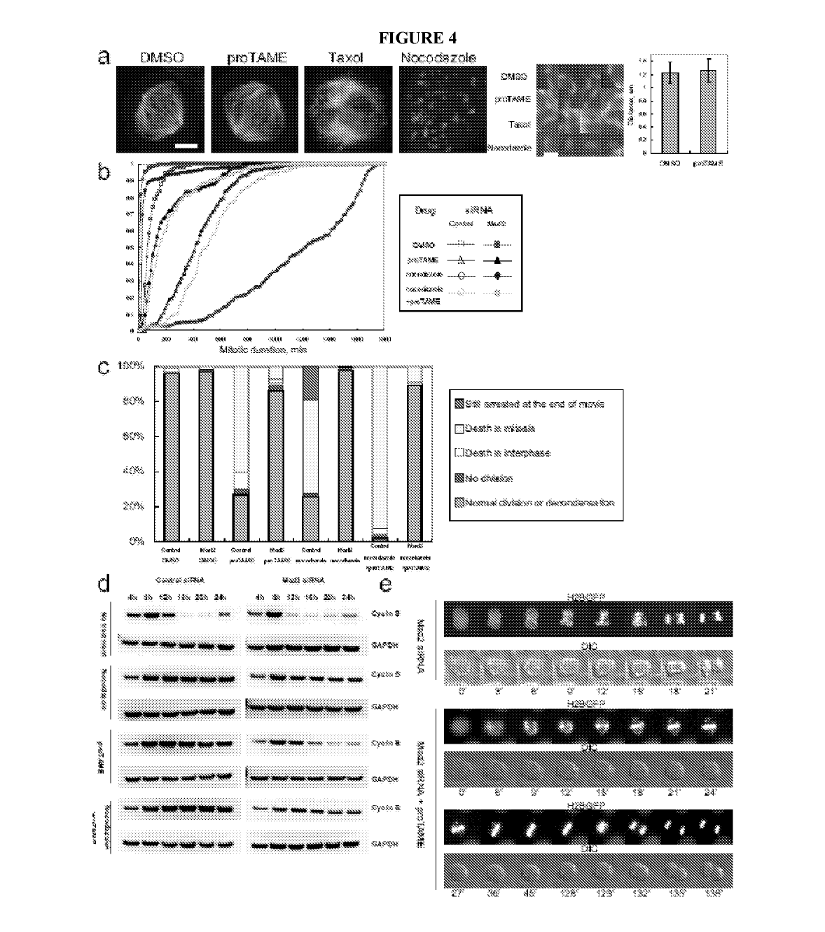

The ability of proTAME to induce a prolonged mitotic arrest and inhibit APC activation in cells, despite producing only a partial reduction of Cdc20 association with the APC, suggested that the SAC may be important for the proTAME-induced mitotic arrest. One possibility is that in addition to its ability to directly bind to the APC, proTAME may nonspecifically perturb microtubules, resulting in SAC activation. Mitotic spindle and kinetochore morphology was therefore examined in HeLa cells arrested in mitosis with proTAME or microtubule inhibitors for 2 h.

Whereas nocodazole and taxol strongly perturbed spindle organization, proTAME had no measurable effects on spindle morphology, spindle-kinetochore attachment, or tension across kinetochores compared to the DMSO control (FIG. 4A), suggesting that the mitotic arrest observed with proTAME treatment is not a consequence of microtubule perturbation.

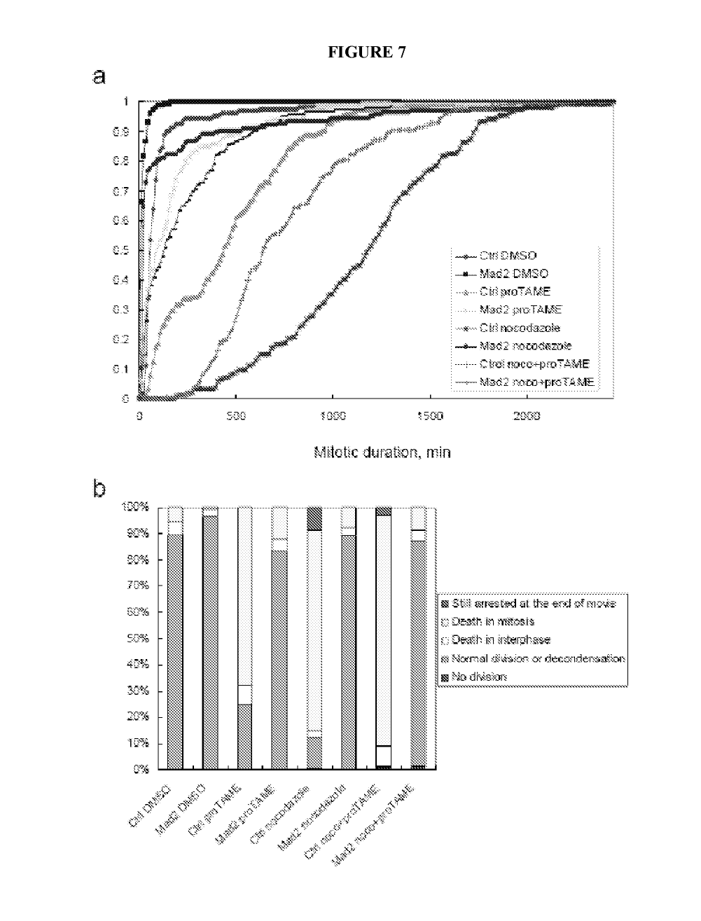

To determine whether proTAME induced-arrest is SAC-dependent, Mad2 expression was knocked down with siRNA in synchronized cells, and the effects of proTAME or nocodazole on mitosis were analyzed by live cell fluorescence imaging. Mad2 knockdown shortened median mitotic duration from 75 minutes to 15 minutes in DMSO-treated cells (FIG. 4B), and shortened nocodazole-induced mitotic arrest from 1230 minutes to 30 minutes (FIG. 4B), indicating that the SAC was fully inactivated. Mad2 knockdown also shortened the duration of proTAME-induced arrest, from 420 minutes to 120 minutes, suggesting that proTAME-induced arrest is partially dependent on the SAC. However, Mad2 knockdown cells remained in mitosis for 2 hours in the presence of proTAME, indicating that a significant portion of the arrest is also SAC-independent (FIG. 4B). In support of our earlier observations, the arrest in Mad2-knockdown cells cannot be a consequence of nonspecific microtubule disruption by proTAME, because nocodazole-induced microtubule disruption arrested Mad2 knockdown cells for only 30 minutes rather than 2 hours. Furthermore, addition of proTAME to nocodazole-treated Mad2 knockdown cells extended mitotic arrest from 30 minutes to 150 minutes, showing that microtubules are not a direct target of proTAME (FIG. 4B). A replicate experiment by DIC imaging of asynchronous cells produced similar results (FIGS. 7A, B).

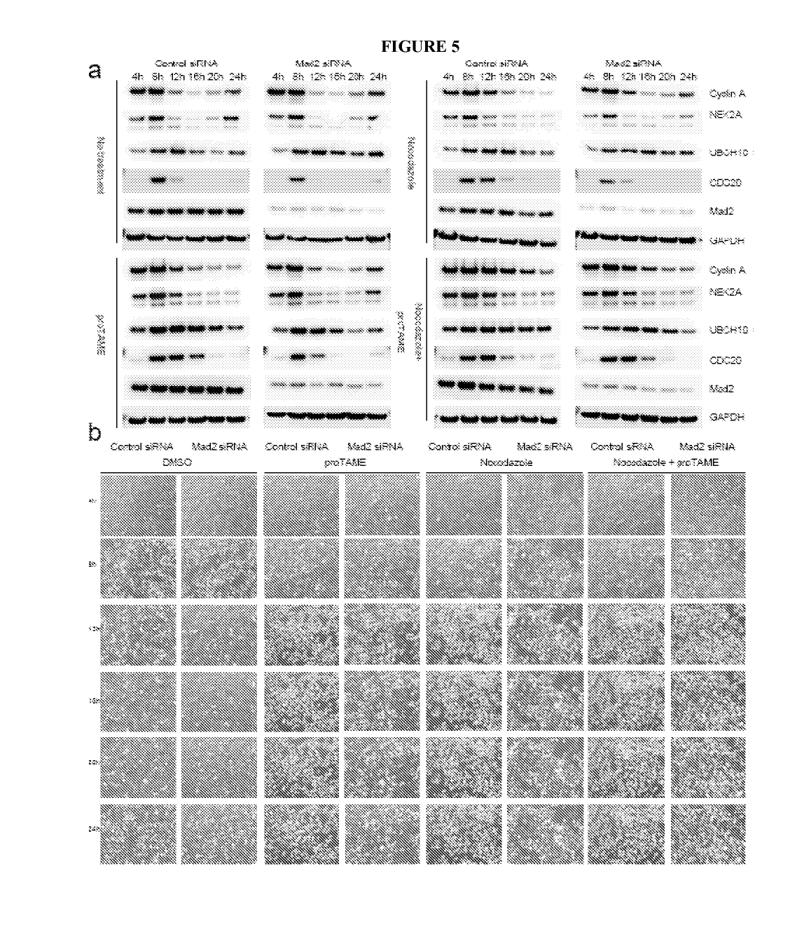

The Mad2-dependence of proTAME arrest was also confirmed by measurement of APC substrate levels in synchronized cells by western blotting (FIG. 4D). RNAi-mediated knockdown reduced Mad2 levels by 80% (FIG. 5A). This was sufficient to inactivate the SAC as measured by phase-contrast imaging (FIG. 5B). Although Mad2 knockdown resulted in efficient override of the mitotic arrest induced by nocodazole, cyclin was not efficiently degraded, as noted by other investigators (Michel, L. S. et al. Nature 409(6818): 355 (2001)), suggesting that microtubules may be required for efficient cyclin B proteolysis in human cells. In contrast, cyclin B was degraded more efficiently when proTAME-treated cells exited mitosis as a consequence of Mad2 depletion (FIG. 4D). These findings indicate that the mechanisms of mitotic exit in the presence of proTAME and nocodazole are distinct. Other APC substrates were efficiently degraded following Mad2 knockdown in the presence of either nocodazole or proTAME (FIG. 5A).

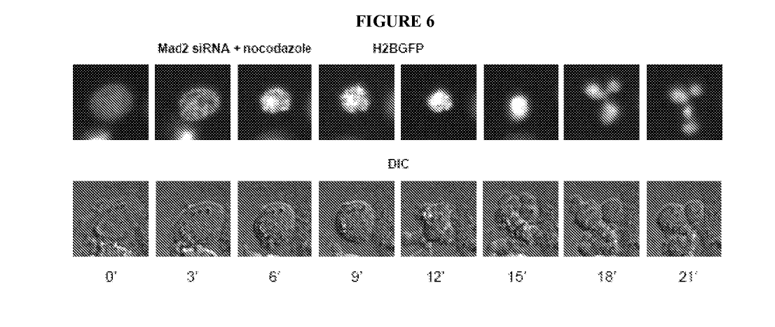

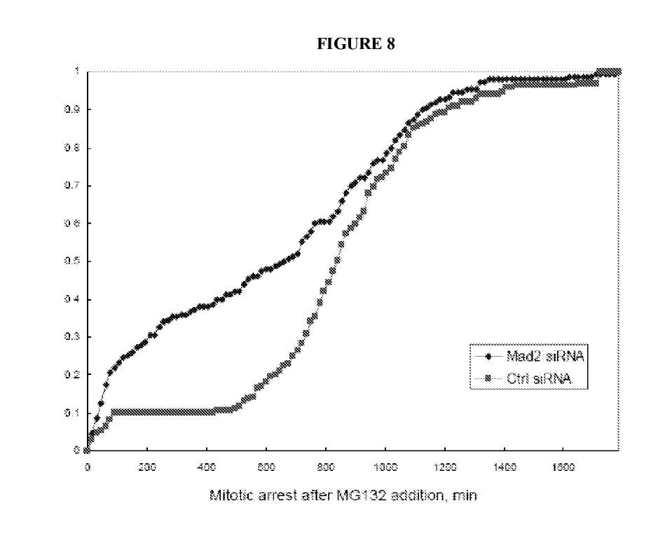

To confirm that proTAME directly inhibits APC activation, it was determined that the compound restores a normal mitosis in cells lacking Mad2. Thus, the effects of proTAME in Mad2 knockdown cells were analyzed in more detail by live cell imaging. Unlike control cells, Mad2 knockdown cells initiate anaphase before establishing a metaphase plate because of unconstrained APC activity (FIG. 4E), and slip out of mitosis quickly in the presence of nocodazole (FIG. 6). In striking contrast, the addition of proTAME allowed Mad2 knockdown cells sufficient time to build a normal metaphase plate followed by initiation of cytokinesis, fully rescuing the mitotic defect caused by Mad2 knockdown (FIG. 4E). Taken together, the data indicate that proTAME is capable of inducing a mitotic arrest with a duration of approximately 2 hours in the absence of Mad2, but sustained mitotic arrest requires the presence of an intact SAC. The data indicate that APC-dependent ubiquitination or proteolysis may be required for inactivation of the SAC. This idea is further supported by the finding that mitotic arrest induced by 3 μM MG132 is also partially dependent on Mad2 (FIG. 8).

The Mad2-dependence of proTAME-induced mitotic arrest in HeLa cells suggests that IR tail binding sites may be important for inactivation of the SAC. Recent work suggests that Cdc20 ubiquitination may be important for inducing dissociation of Cdc20 from Mad2. Given that elimination of the IR tail stabilizes Cdc20 in budding yeast, it is possible that association of the IR tail with the APC is required to trigger Cdc20 ubiquitination. The IR tail of Cdc20 may be especially critical for the ability of the APC to capture Mad2-Cdc20 complexes, ubiquitinate

them, and induce their dissociation. By blocking IR-binding sites on the APC, proTAME may prevent APC from capturing Mad2-Cdc20 complexes and thereby inhibit SAC inactivation. In addition, APC-dependent proteolysis may also contribute to SAC inactivation, as we observed that the mitotic arrest induced by MG132 is also partially Mad2 dependent. Candidate substrates could include Mps1, which is a target of the APC in budding yeast.

### Example 3

### ProTAME in Mammalian Cells Versus Xenopus Extracts

proTAME is less effective at inhibiting Cdc20 association with the APC in mammalian cells than in Xenopus extracts. Although it is possible that the APC-Cdc20 dissociation pathway may not be as active in mammalian cells, an active SAC in mammalian cells may counter the ability of TAME to induce Cdc20 dissociation. For example, SAC complexes, such as the MCC, may provide an alternative pathway for loading Cdc20 onto the APC that does not require the Cdc20 IR tail. It is also possible that the binding of checkpoint proteins to Cdc20 may shield it from factors that promote Cdc20 dissociation. Because the SAC is not active in Xenopus egg extract, such alternative loading or shielding mechanisms would not

be operative, explaining why TAME is capable of inducing complete dissociation of Cdc20 from APC in the Xenopus system but not in mammalian cells.

### Example 4

### Pharmacologic Inhibition of the Anaphase-Promoting Complex (APC) Induces a Spindle Checkpoint Dependent Mitotic Arrest in the Absence of Spindle Damage

Microtubule inhibitors are important cancer drugs that induce mitotic arrest by activating the spindle assembly checkpoint (SAC), which in turn inhibits the ubiquitin ligase activity of the Anaphase-Promoting Complex (APC). This invention is based, at least in part, upon the discovery of a small molecule, Tosyl-L-Arginine Methyl Ester (TAME), which binds to the APC and prevents its activation by Cdc20 and Cdh1. The invention provides a prodrug of TAME that arrests cells in metaphase without perturbing the spindle. Nonetheless, this arrest is dependent on the SAC. Metaphase arrest induced by a proteasome inhibitor is also SAC-dependent, suggesting that APC-dependent proteolysis is required to inactivate the SAC. The mutual antagonism between the APC and the SAC yields a positive feedback loop that amplifies the ability of TAME to induce mitotic arrest.

The Anaphase-Promoting Complex (APC) is required for mitotic exit, making the APC a potential new target for antimitotic chemotherapy. TAME is the first small molecule inhibitor of the APC. The methods of the invention are used to develop and therapeutic uses for a cell-permeable derivative, proTAME. Treatment of cells with proTAME causes a surprisingly robust mitotic arrest because APC-dependent proteolysis is required for inactivation of the spindle assembly checkpoint (SAC). In contrast, SAC-activating compounds, such as microtubule inhibitors, do not suppress APC activity as completely. As a result, cells rely on continued protein synthesis to maintain mitotic arrest, providing an explanation for the known variability in cellular response to microtubule inhibitors. Thus, direct APC inhibitors may provide a more uniform and specific method for inducing mitotic arrest.

TAME is a small molecule that inhibits APC activation by preventing Cdc20 binding. A cell-permeable prodrug (proTAME) induces mitotic arrest and cell death. APC-dependent proteolysis is required for spindle-assembly checkpoint inactivation. ProTAME exploits mutual antagonism between the SAC and APC to block mitotic exit.

Microtubule inhibitors such as taxanes and the vinca alkaloids represent one of the most important classes of cancer drugs, used in the treatment of breast, ovarian, and lung cancer (Montero, A., et al. (2005). Lancet Oncol 6, 229-239). However, the response of cells to microtubule inhibitors is highly variable (Brito, D. A., et al. (2008). J Cell Biol 182, 623-629; Gascoigne, K. E., and Taylor, S. S. (2008). Cancer Cell 14, 111-122; Orth, J. D., et al. (2008). Mol Cancer Ther 7, 3480-3489; Shi, J., et al. (2008). Cancer Res 68, 3269-3276), potentially compromising clinical efficacy. How these drugs cause cell death remains unclear, but induction of mitotic arrest appears to be a key aspect of the mechanism (Bekier, M. E., et al. (2009). Mol Cancer Ther 8, 1646-1654; Huang, H. C., et al. (2009). Cancer Cell 16, 347-358). By perturbing the mitotic spindle, these drugs activate the Spindle Assembly Checkpoint (SAC), which delays mitotic exit by inhibiting the ubiquitin ligase activity of the Anaphase-Promoting Complex/Cyclosome (APC). In principle, a compound that directly inhibits APC-dependent proteolysis should arrest cells in mitosis without causing side effects that result from microtubule inhibition, such as peripheral neuropathy.

The APC is the most complex ubiquitin ligase known, consisting of more than 11 subunits. The activator proteins Cdh1 and Cdc20 bind to the APC at different cell cycle stages to stimulate APC-dependent ubiquitination of substrates and their subsequent destruction by the 26S proteasome (Peters, J. M. (2006). Nat Rev Mol Cell Biol 7, 644-656). The activators assist in recruitment of APC substrates and may also stimulate the catalytic activity of the ligase (Burton, J. L., et al. (2005). Mol Cell 18, 533-542; Kimata, Y., et al. (2008). Mol Cell 32, 576-583; Pfleger, C. M., et al. (2001). Genes Dev 15, 2396-2407). During G1, Cdh1 binds to the APC to promote degradation of APC substrates to keep mitotic cyclin-dependent kinase activity low. The initiation of anaphase and exit from mitosis instead require Cdc20-dependent ubiquitination of APC substrates such as securin and mitotic cyclins (Kraft, C., et al. (2003). EMBO J. 22, 6598-6609; Kramer, E. R., et al. (2000). Mol Biol Cell 11, 1555-1569; Yu, H. (2007). Mol Cell 27, 3-16). Prior to anaphase, the ability of APC-Cdc20 to ubiquitinate certain substrates is inhibited by the SAC (Musacchio, A., and Salmon, E. D. (2007). Nat Rev Mol Cell Biol 8, 379-393). Unattached kinetochores catalyze the formation of an inhibitory protein complex, containing the proteins Mad2, BubR1 and Bub3 (Sudakin, V., et al. (2001). J Cell Biol 154, 925-936), that sequesters Cdc20 or interferes with its ability to activate the APC. Attachment of kinetochores to the mitotic spindle diminishes their ability to generate an inhibitory signal. Subsequently, the SAC-inhibited APC-Cdc20 complex is activated, through a process that may require APC-dependent ubiquitination (Reddy, S. K., et al. (2007). Nature 446, 921-925; Stegmeier, F., et al. (2007). Nature 446, 876-881), though the mechanism remains incompletely understood (Nilsson, J., et al. (2008). Nat Cell Biol 10, 1411-1420).

Because the APC regulates multiple cell cycle events, it is not clear whether pharmacological inhibition of its activity will lead to selective or prolonged arrest in mitosis as is the case with microtubule inhibitors. Proteasome inhibitors can block APC-dependent proteolysis without perturbing the mitotic spindle (Famulski, J. K., and Chan, G. K. (2007). Curr Biol 17, 2143-2149), but they also inhibit the degradation of many other substrates of the ubiquitin-proteasome system, and therefore also cause cell cycle arrest during interphase (Wojcik, C., et al. (1996). Eur J Cell Biol 70, 172-178). It may be difficult to achieve mitotic arrest by pharmacologic APC inhibition, as RNAi approaches indicate that Cdc20 expression must be reduced to a very low level to induce a mitotic arrest (Huang, H. C., et al. (2009). Cancer Cell 16, 347-358; Wolthuis, R., et al. (2008). Mol Cell 30, 290-302). Even when the SAC is strongly activated by a dose of microtubule inhibitor that completely depolymerizes microtubules, a fraction of cells escape mitotic arrest due to residual APC activity (Brito, D. A., and Rieder, C. L. (2006). Curr Biol 16, 1194-1200) suggesting that the SAC cannot fully inhibit the APC during mitosis. For this reason, microtubule inhibitors may suffer from limited effectiveness because some cells escape mitotic arrest before dying (Bekier, M. E., et al. (2009). Mol Cancer Ther 8, 1646-1654; Huang, H. C., et al. (2009). Cancer Cell 16, 347-358). Whether an APC inhibitor can better extinguish APC activity and induce a more persistent mitotic arrest is therefore an important question in contemplating development of APC inhibitors as a therapeutic strategy for cancer. Here the first small molecule inhibitor of the APC is described and the compound is used as a tool to reveal reciprocal antagonism between the APC and the SAC. The existence of this regulatory relationship makes direct APC inhibition a particularly effective approach for inducing mitotic arrest.

TAME Inhibits APC Activation by Perturbing Activator Protein Binding.

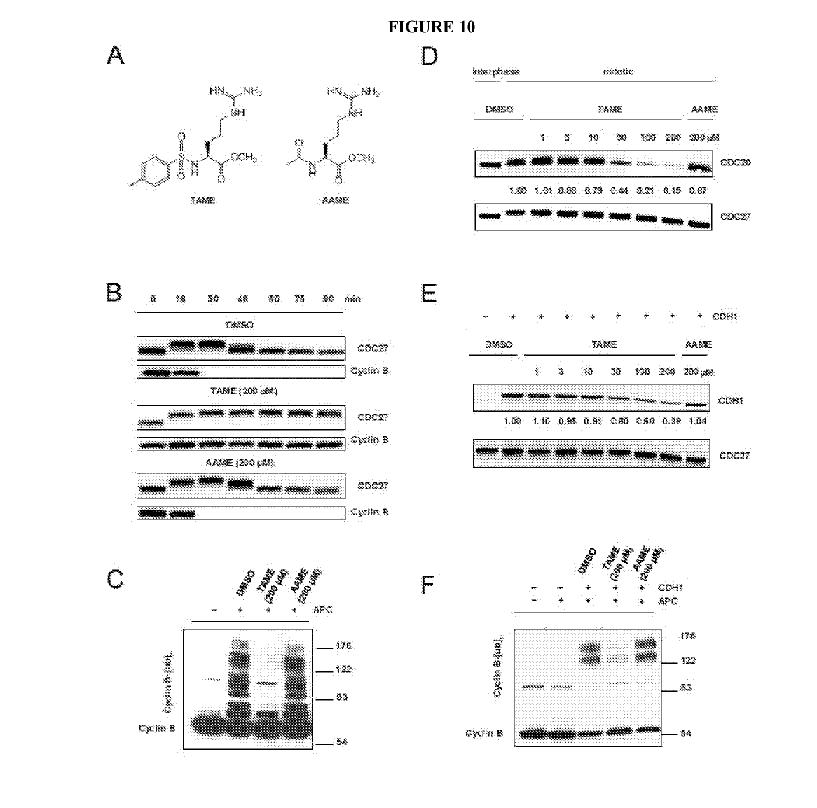

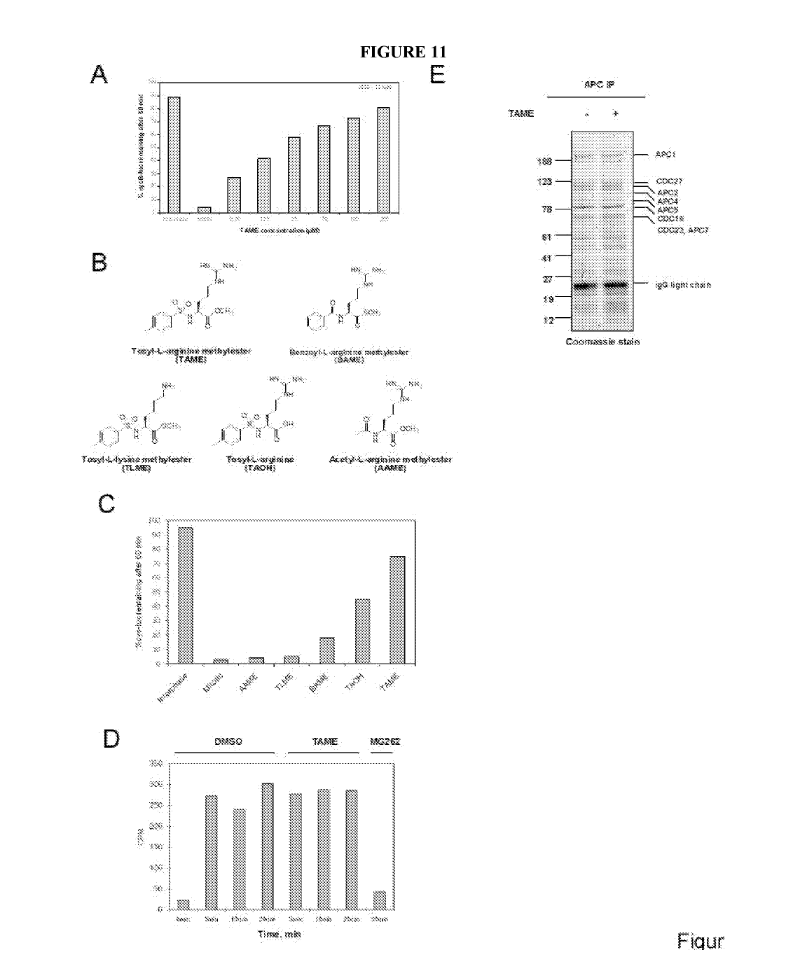

TAME (FIG. 10A) was identified as an inhibitor of cyclin proteolysis in mitotic Xenopus egg extract (IC₅₀ of 12 μM; FIG. 11A), but its mechanism of action has remained unknown. TAME also inhibited cyclin degradation in interphase extract activated by exogenous Cdh1, but had no effect on SCF-dependent proteolysis of β-catenin-luciferase (Verma, R., et al. (2004). Science 306, 117-120), indicating that it is not a general inhibitor of the ubiquitin-proteasome system. Testing of TAME derivatives indicated that the tosyl group, arginine, and the methyl ester are each important for activity (FIGS. 11B and 11C). A derivative, Acetyl-L-Arginine Methyl Ester (AAME; FIG. 10A) showed only low activity, and was therefore used as a negative control in subsequent experiments. When added to interphase extract treated with recombinant cyclin B1/cdc2 complex, TAME, but not AAME, arrested the extract in mitosis, with stable cyclin B1 and phosphorylated Cdc27 (FIG. 10B). Another APC substrate, cyclin A, was also stabilized by TAME in Xenopus extract. TAME had no effect on the ability of Xenopus extract to degrade cyclin B1 that had been preubiquitinated in vitro (FIG. 11D), indicating that TAME does not inhibit the proteasome or its ability to recognize ubiquitinated substrates.

Because the SAC is not active in Xenopus extracts (Minshull, J., et al. (1994). Cell 79, 475-486), these findings suggested that TAME might inhibit cyclin proteolysis by directly inhibiting the APC. Indeed, when TAME was added to mitotic Xenopus extract during APC isolation, the APC showed a dramatic loss of activity in a reconstituted ubiquitination reaction (FIG. 10C). These results suggested that adding TAME to extract might alter APC composition, inactivating the complex. Consistent with this hypothesis, TAME addition to extract reduced Cdc20 association with the APC in a dose-dependent manner (FIG. 10D), but did not otherwise affect APC composition (FIG. 11E). TAME also inhibited the binding of Cdh1 to APC when Cdh1 and TAME were added together to interphase extract (FIG. 10E). The reduction in Cdh1 binding was accompanied by a reduction in APC activation (FIG. 10F). These findings suggested that TAME might block APC activation by perturbing the interaction between APC and its activator proteins Cdc20 or Cdh1.

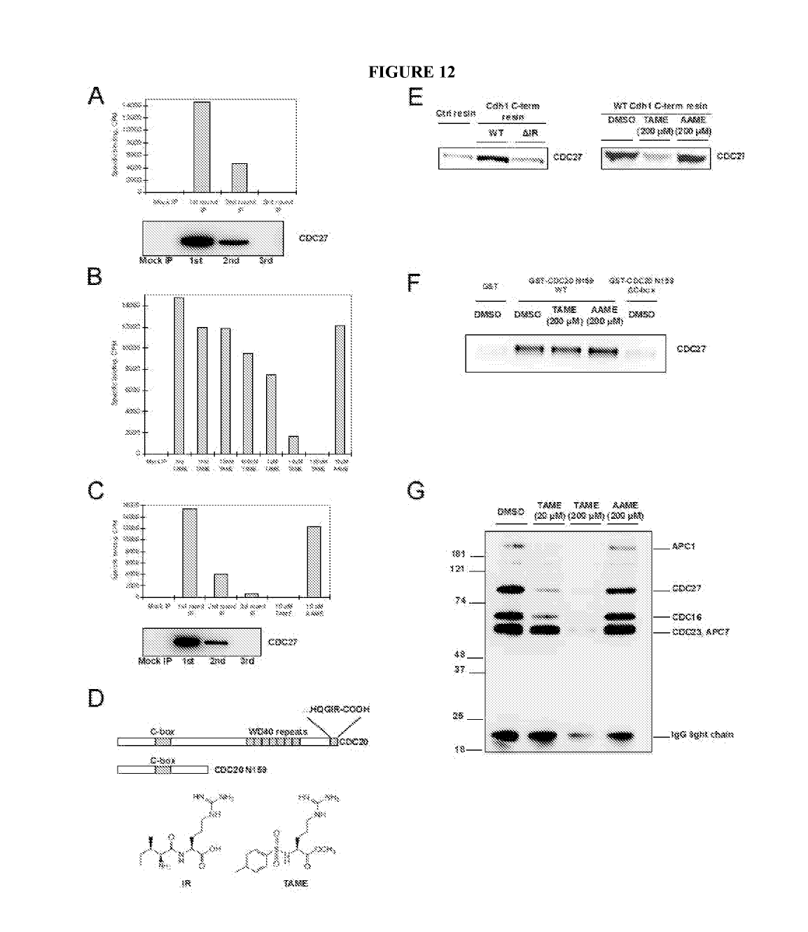

To understand how TAME disrupts the interaction between the activator proteins and the APC, it was first determined whether TAME binds to the APC. ³H-TAME was added to interphase Xenopus extract, or to extract immunodepleted of APC, and then isolated residual APC with Cdc27 antibodies and measured the amount of radioactivity associated with the beads. Binding of ³H-TAME correlated with the amount of immunoprecipitated Cdc27 (FIG. 12A). Unlabeled TAME competitively inhibited the binding of ³H-TAME, whereas AAME did not (FIG. 12B). Other TAME derivatives competed with ³H-TAME for APC binding in a manner that correlated with their ability to inhibit cyclin-luciferase proteolysis in Xenopus extract (FIG. 13A). A similar approach demonstrated that TAME binds to human APC isolated from HeLa cells (FIG. 12C). Together these findings indicate that the binding of TAME to the APC might explain the ability of TAME to perturb activator protein association.

To understand how TAME disrupts activator binding to the APC, it was determined whether TAME could inhibit the interaction of APC with motifs of the activator proteins that have been implicated in APC binding, including the C-box (Schwab, M., et al. (2001). EMBO J. 20, 5165-5175) and the C-terminal isoleucine-arginine (IR) tail (FIG. 12D) (Burton, J. L., et al. (2005). Mol Cell 18, 533-542; Vodermaier, H. C., et al. (2003). Curr Biol 13, 1459-1468). Because TAME structurally resembles the IR tail of Cdc20 and Cdh1 (FIG. 12D), it was hypothesized that TAME might bind to the APC in the same site normally occupied by the IR tail. Previous work has demonstrated that a C-terminal 20 amino acid peptide derived from Cdh1 (“IR peptide”) is sufficient to isolate Xenopus APC from interphase extract (Vodermaier, H. C., et al. (2003). Curr Biol 13, 1459-1468). This finding was confirmed and it was further discovered that TAME, but not AAME, was sufficient to block APC recruitment by the IR peptide (FIG. 12E). In contrast, TAME had no effect on recruitment of APC from mitotic extract by an N-terminal fragment of Cdc20 containing only the C-box interaction motif (FIG. 12F), indicating that TAME specifically inhibits the IR-tail-dependent interaction.

The APC subunits Cdc27 and APC7 have been implicated in binding of the IR tail of Cdh1 to the APC (Matyskiela, M. E., and Morgan, D. O. (2009). Mol Cell 34, 68-80; Vodermaier, H. C., et al. (2003). Curr Biol 13, 1459-1468). To determine whether TAME could competitively inhibit the binding of the IR-tail to these proteins, the IR peptide was conjugated to a photo-affinity reagent and performed crosslinking studies with APC immunopurified from interphase Xenopus extract. Four proteins known to exist in an APC subcomplex, namely Cdc27, Cdc16, Cdc23 and Apc7, were crosslinked in an IR-dependent manner that could be competed by excess unlabeled IR peptide (FIGS. 13B and 13C). At low concentration (20 μM), TAME efficiently inhibited crosslinking of the IR peptide to Cdc27 and Cdc16 but only slightly reduced crosslinking to Cdc23 and Apc7 (FIG. 12G). At high concentration (200 μM), TAME strongly inhibited crosslinking to all APC subunits (FIG. 12G). Together these findings support the hypothesis that TAME binds to APC subunits that recruit the IR tail, thereby preventing activator proteins from associating with the APC.

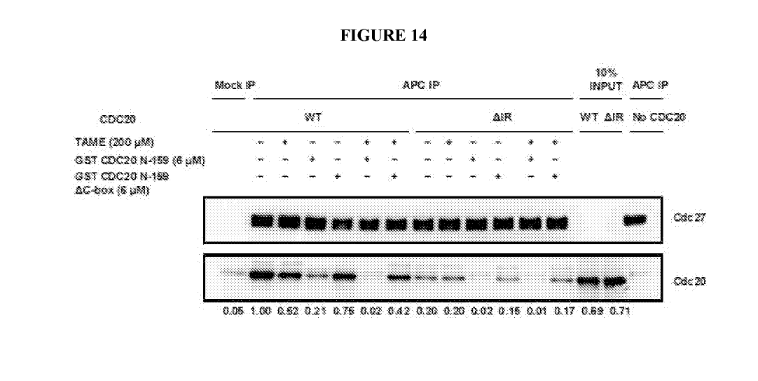

To confirm that TAME specifically antagonizes IR-tail dependent interactions between Cdc20 and the APC, the ability of TAME to inhibit the binding of Cdc20 to the APC was tested in a reconstituted system. APC was purified from mitotic Xenopus extracts and washed with high salt to remove most Cdc20. Purified mitotic APC was then incubated in reticulocyte lysate expressing wild-type or mutant Cdc20, and Cdc20 binding to APC was measured by co-immunoprecipitation. It was determined that efficient binding of Cdc20 to the APC under these conditions indeed requires the IR-tail, as a mutant lacking these two residues (Cdc20ΔIR) did not bind as efficiently to the APC (FIG. 14). TAME also strongly reduced Cdc20 binding to the APC under these conditions (FIG. 14). Importantly, addition of TAME had no further effect on binding of the Cdc20ΔIR mutant, confirming that TAME does not perturb other interactions between Cdc20 and the APC.

TAME addition or IR-tail deletion was not sufficient to fully inhibit Cdc20 association under these conditions. It was hypothesized that other interactions, such as C-box-dependent binding, might promote Cdc20 association with the APC, thereby masking the effect of TAME addition or IR-tail deletion. Consistent with this hypothesis, the results indicated that addition of a C-box-containing N-terminal fragment of Cdc20 could competitively inhibit binding of full-length Cdc20 to the APC (FIG. 14). In the presence of the C-box fragment, addition of TAME or deletion of the IR-tail was sufficient to completely suppress Cdc20 association with the APC. These results indicate that both C-box-dependent and IR-tail-dependent interactions are important for Cdc20 binding in these conditions, and that TAME specifically disrupts the IR-dependent interaction. Thus, the target of TAME is the APC, and it inhibits APC activation by interfering specifically with IR-tail dependent interactions between Cdc20 or Cdh1 and the APC.

A TAME Prodrug Inhibits APC-Cdh1 Activation in Cells.

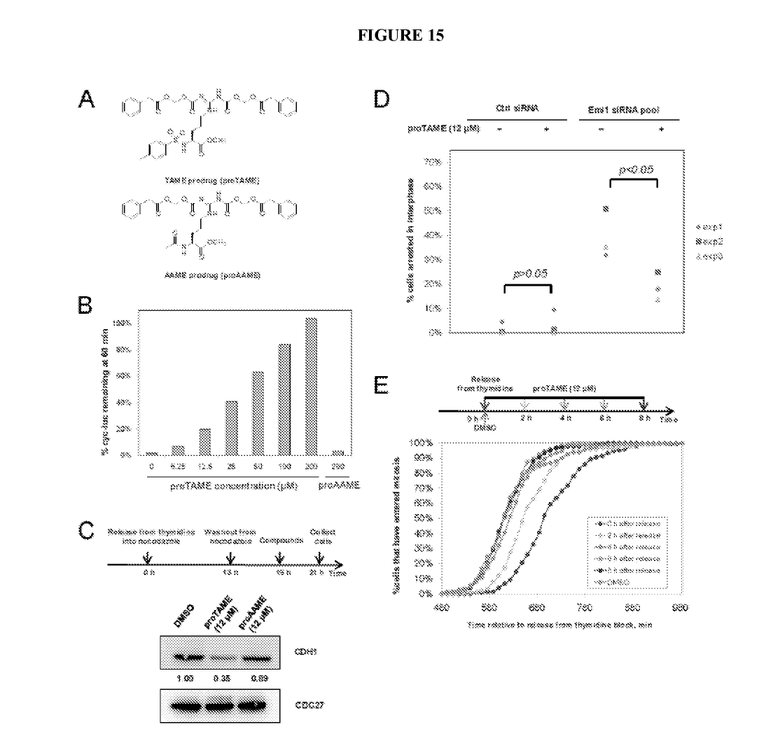

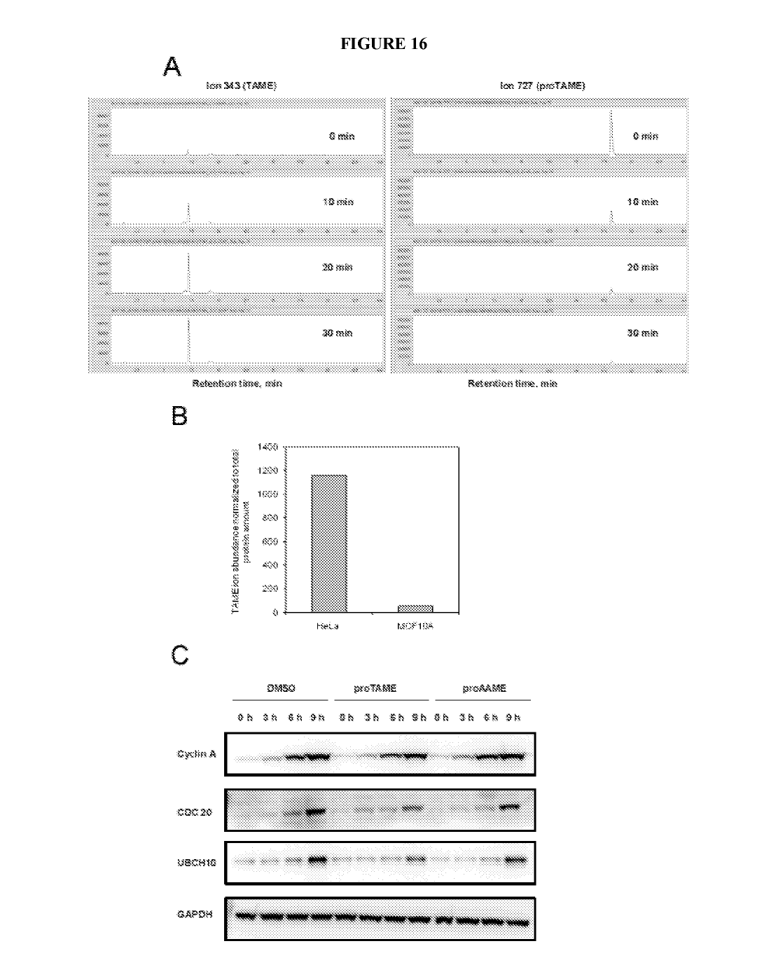

Having established the mechanism by which TAME inhibits APC activation in Xenopus extract, it was next determined whether TAME inhibits APC activation in human cells. Because TAME is not cell permeable, a TAME prodrug (proTAME) was synthesized, and its control compound proTAME, by modifying the guanidino group to produce an N,N′-bis(acyloxymethyl carbamate) derivative (FIG. 15A). Such prodrugs can be processed by intracellular esterases to yield the parent compound. In Xenopus extract, proTAME was indeed rapidly converted to TAME (FIG. 16A), which efficiently inhibited cyclin B-luciferase proteolysis (FIG. 15B). ProTAME was also activated efficiently in HeLa cells, but not in MCF10A cells (FIG. 16B).

It was next examined whether proTAME could inhibit association of Cdh1 with the APC in cells. HeLa cells expressing H2B-GFP were released from a nocodazole block and 12 μM of proTAME were added after cells had entered G1, when the APC is activated by Cdh1. Addition of proTAME inhibited Cdh1 association with the APC (FIG. 15C) but proAAME did not. However, proTAME was not sufficient to cause premature accumulation of endogenous APC substrates in G1 or S phase (FIG. 16C). During S phase, when APC substrates are known to be expressed, the effect of proTAME may be masked by Emi1-dependent inhibition of APC-Cdh1 (Hsu, J. Y., et al. (2002). Nat Cell Biol 4, 358-366). To test this idea, cells were depleted of Emi1, which leads to degradation of APC substrates and prevents mitotic entry (Hsu, J. Y., et al. (2002). Nat Cell Biol 4, 358-366). It was confirmed that Emi1 depletion prevents mitotic entry, and found that addition of 12 μM proTAME substantially rescued the mitotic entry defect caused by depletion of Emi1 (FIG. 15D). Therefore, proTAME is capable of inhibiting APC-Cdh1 function in cells.

Previous studies have shown that knockdown of Cdh1 induces prolonged S-phase and mitotic entry delay in human cells (Engelbert, D., et al. (2008). Oncogene 27, 907-917; Sigl, R., et al. (2009). J Cell Sci 122, 4208-4217). Consistent with these findings, proTAME caused a 2 hour delay in mitotic entry when added during release from a double thymidine block (FIG. 15E). However, adding proTAME 6 hour or later after release did not delay mitotic entry (FIG. 15E), suggesting the delay may be a consequence of inhibiting APC-Cdh1 in S-phase. These findings indicate that although proTAME can inhibit APC-Cdh1 activation, it has only modest effects on cell cycle progression during interphase.

ProTAME Induces Mitotic Arrest in the Absence of Spindle Damage.

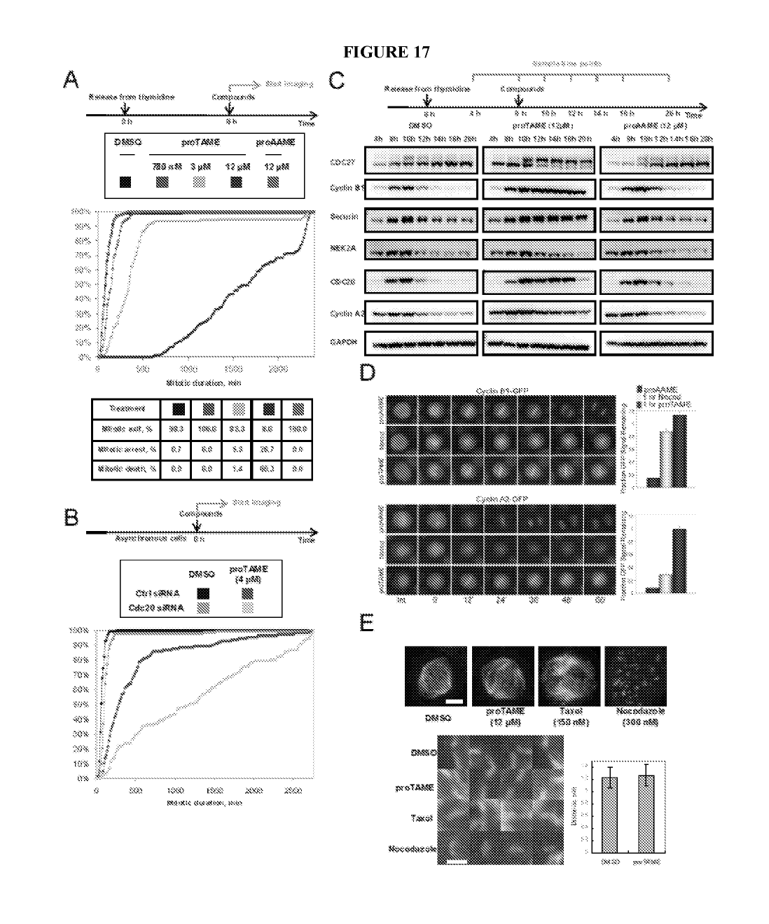

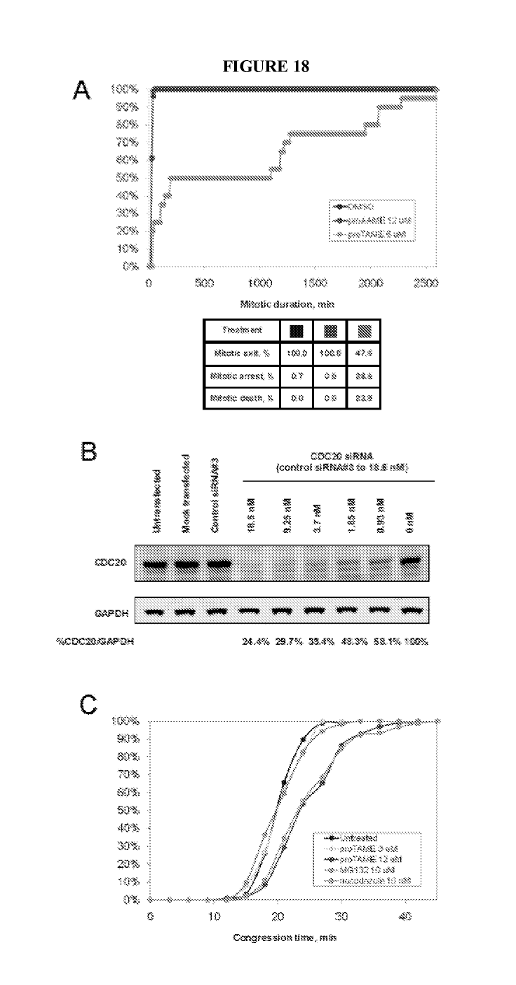

To examine effects of proTAME treatment on mitosis, HeLa H2B-GFP cells were released from a double thymidine block and added proTAME 8 h after release, a time at which proTAME addition does not delay mitotic entry (FIG. 15E). Mitotic duration was then measured by time-lapse imaging. Cells treated with low doses of proTAME (780 nM or 3 μM) remained in metaphase for up to several hours, but then proceeded through a normal anaphase, whereas cells treated with 12 μM proTAME arrested in metaphase and subsequently died (FIG. 17A). In contrast, treatment of cells with 12 μM proAAME had no effect. ProTAME greatly increased mitotic duration in asynchronous hTERT-RPE1 cells as well, as 6 μM proTAME increased median mitotic duration to over 8 h, compared to 24 min in proAAME-treated cells (FIG. 18A). ProTAME had no effect at similar doses in MCF10a cells, because the prodrug was not efficiently activated (FIG. 16B).

If proTAME blocks mitotic progression by disrupting the APC-Cdc20 interaction, then reducing Cdc20 expression should enhance the mitotic exit delay induced by proTAME treatment. In control-transfected cells, 4 μM proTAME increased mitotic duration from 1.0 h to 4.8 h (FIG. 17B). However, when Cdc20 levels were reduced by 50% using siRNA-mediated knockdown (FIG. 18B), proTAME prolonged mitotic duration to 19.4 h (FIG. 17B). This effect was synergistic, because Cdc20 knockdown by itself only increased mitotic duration to 1.6 hours. These results show that reducing the expression of Cdc20 strongly sensitizes cells to the effect of proTAME, consistent with the APC-Cdc20 interaction as the relevant target of the compound.

The effect of proTAME treatment on degradation of APC substrates was investigated. Because the SAC does not stabilize all APC substrates during mitosis, some substrates such as cyclin A2, Cdc20, and Nek2A are degraded in cells treated with microtubule inhibitors (den Elzen, N., and Pines, J. (2001). J Cell Biol 153, 121-136; Hayes, M. J., et al. (2006). Nat Cell Biol 8, 607-614; Nilsson, J., et al. (2008). Nat Cell Biol 10, 1411-1420). In contrast, substrates such as cyclin B1 and securin are stabilized by SAC activation. If proTAME directly inhibits APC activation, then it would stabilize all APC substrates during mitosis, not just those whose stability depends on the SAC. Consistent with this hypothesis, cells treated with proTAME accumulated cyclin A2, Cdc20 and Nek2A in addition to cyclin B1 and securin (FIG. 17C). These results were confirmed in live cell imaging experiments, where proTAME stabilized cyclinA2-GFP but the microtubule depolymerizer nocodazole did not (FIG. 17D). Interestingly, proTAME treatment led to greater accumulation of cyclin B1-GFP than nocodazole treatment, suggesting that proTAME inhibited APC activation more effectively than a SAC-activating compound, consistent with the ability of proTAME to directly inhibit APC activation.

The effects of proTAME treatment on mitotic spindle morphology and chromosome congression were assessed and compared to the effects of treatment of cells with microtubule inhibitors. Compared to DMSO-treated cells, treatment of asynchronous HeLa cells with 12 μM proTAME for 2 h yielded no measurable differences in mitotic spindle morphology or inter-kinetochore distance, which reflects development of proper kinetochore tension (FIG. 17E). In contrast, treatment of cells with nocodazole or taxol for 2 h strongly perturbed spindle organization (FIG. 17E). In live cell imaging experiments, treatment of cells with 3 μM proTAME or 10 μM MG132 caused no delay in chromosome congression (FIG. 18C). Treatment of cells with 10 nM nocodazole or 12 μM proTAME caused a similar mild congression delay of 6 min (FIG. 18C), but these treatments produced contrasting effects on the metaphase plate. In cells treated with 10 nM nocodazole, the metaphase plate appeared loose and was prone to bending, whereas in cells treated with 12 μM proTAME the metaphase plate appeared tight and did not bend. Importantly, 10 nM nocodazole prolonged mitosis by only 20 min (data not shown), whereas 12 μM proTAME induced a mitotic arrest of over 28 h (FIG. 17A). Thus, the mild delay in congression is not sufficient to explain the ability of proTAME to arrest cells in mitosis. ProTAME induces a mitotic arrest by perturbing the APC-Cdc20 interaction rather than by stimulating the SAC through interfering with chromosome attachment to the mitotic spindle.

ProTAME-Induced Mitotic Arrest is SAC-Dependent.

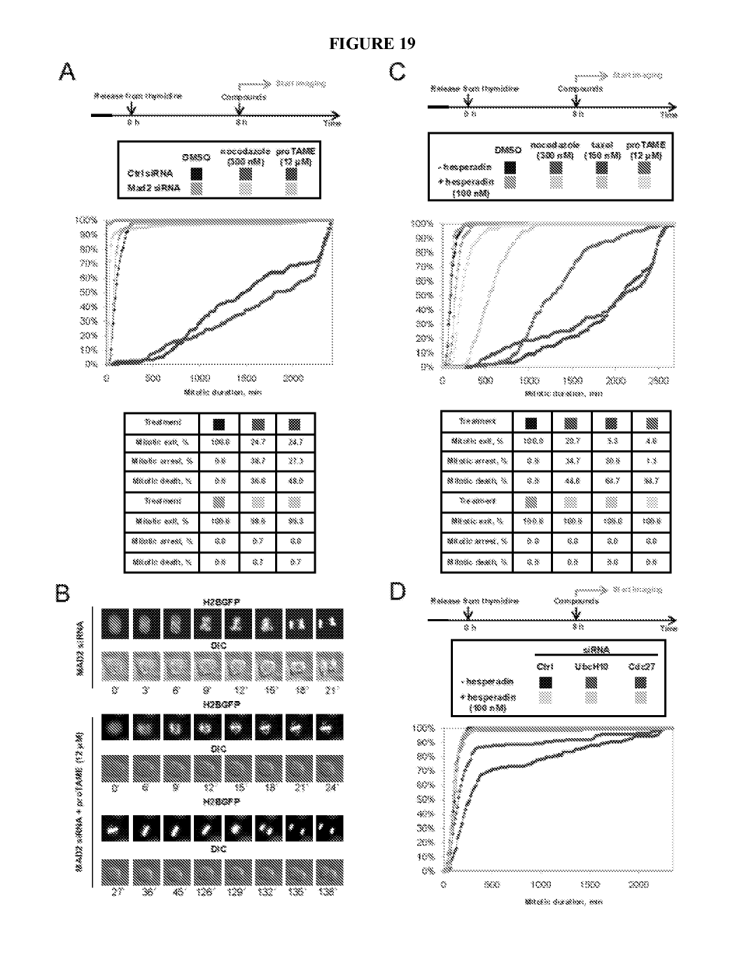

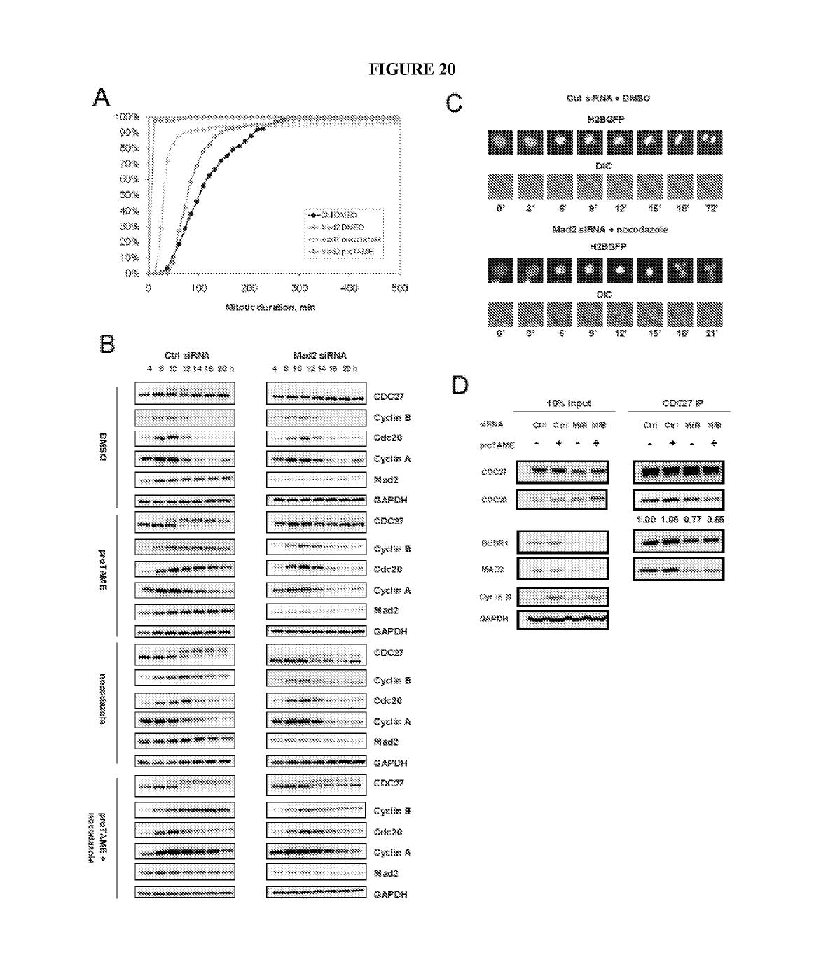

Because TAME directly inhibits the APC, and causes arrest in metaphase with kinetochores that develop tension, it was hypothesized that the proTAME-induced mitotic arrest in human cells would be independent of the SAC. Therefore, unexpectedly, it was determined that the SAC is in fact essential for the prolonged mitotic arrest of cells treated with proTAME. In double-thymidine synchronized cells, Mad2 knockdown greatly shortened the duration of proTAME-induced arrest, from 24.6 h to 1.4 h (FIGS. 19A and 20A). As expected, Mad2 knockdown abrogated nocodazole-induced arrest, shortening the average mitotic duration from 30 h to 0.6 h. The Mad2-dependence of the proTAME-induced arrest was confirmed by measurement of APC substrate levels in synchronized cells (FIG. 20B). These findings indicate that prolonged mitotic arrest induced by proTAME depends strongly on the SAC.

These experiments also revealed the ability of proTAME to delay mitotic exit independent of Mad2, as expected based on TAME's ability to directly inhibit APC activation. In Mad2 knockdown cells, proTAME treatment increased median mitotic duration from 12 min to 84 min (FIGS. 19A and 20A). Strikingly, this mitotic delay was sufficient to give Mad2 knockdown cells enough time to build a normal metaphase plate before initiating anaphase, rescuing the chromosome segregation defect caused by Mad2 knockdown (FIGS. 19B and 20C). The ability of proTAME to restore normal mitotic division in cells depleted of Mad2 highlights the ability of proTAME to directly inhibit APC activation, and further demonstrates that proTAME is unlikely to perturb microtubules or interfere with kinetochore function.

Whereas TAME caused substantial reduction of Cdc20 binding to the APC in Xenopus extracts, proTAME treatment did not cause substantial dissociation of Cdc20 from APC during mitotic arrest in HeLa cells. This might be a consequence of an active SAC pathway that may promote IR-tail independent binding of Cdc20 to the APC. Therefore, the effect of depleting SAC proteins on the ability of proTAME to disrupt the APC-Cdc20 interaction was examined. Indeed, when HeLa cells were arrested in mitosis by expression of nondegradable cyclin B, proTAME induced significant dissociation of Cdc20 from the APC, but only if SAC proteins were depleted by RNAi (FIG. 20D). These results show that a proTAME-induced mitotic arrest occurs without substantial dissociation of Cdc20 from the APC, as a consequence of persistent activity of the SAC.

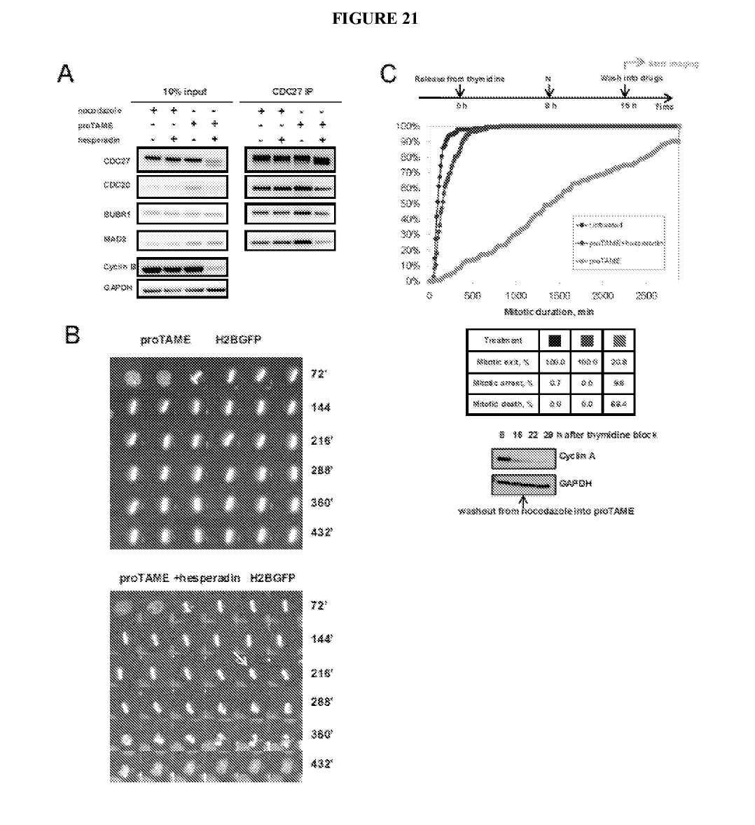

To further understand the SAC-dependence of the proTAME arrest, SAC signaling was pharmacologically inactivated by treating cells with hesperadin (Hauf, S., et al. (2003). J Cell Biol 161, 281-294), an inhibitor of Aurora B kinase. This kinase phosphorylates proteins at kinetochores that are not under tension, leading to destabilization of microtubule-kinetochore interactions and activation of the SAC (Biggins, S., and Murray, A. W. (2001). Genes Dev 15, 3118-3129; Cheeseman, I. M., et al. (2006). Cell 127, 983-997; DeLuca, J. G., et al. (2006). Cell 127, 969-982). However, recent work using phosphospecific antibodies that recognize Aurora B substrates indicates that kinetochore proteins remain phosphorylated at a basal rate during metaphase (Welburn, J. P., et al. (2010). Mol Cell 38, 383-392). It was hypothesized that this basal rate of Aurora B-dependent phosphorylation may produce a persistent SAC signal during metaphase that may contribute to the proTAME-induced arrest. Three observations supported this hypothesis. First, hesperadin treatment dramatically shortened proTAME-induced mitotic arrest (FIG. 19C), and led to dissociation of Mad2 and BubR1 from the APC in proTAME-arrested cells (FIG. 21A). As expected, hesperadin also substantially shortened taxol-induced mitotic arrest, with a less pronounced effect on nocodazole-induced arrest (FIG. 19C). Second, treatment of proTAME-arrested metaphase cells with hesperadin caused deformation of the metaphase plate before cells exited mitosis, suggesting that ongoing Aurora B-dependent phosphorylation is required to maintain proper kinetochore-microtubule attachment in metaphase (FIG. 21B). Third, knockdown of the APC component Cdc27 or the APC-specific E2 UbCH10 caused a mitotic delay that could be completely suppressed by hesperadin treatment (FIG. 19D). Together, these experiments support the idea that the SAC remains active in metaphase, despite the presence of properly attached chromosomes, and that SAC signaling is important for the prolonged mitotic arrest induced by APC inhibition.

One possible explanation for the SAC-dependence of the proTAME arrest is that proTAME stabilizes APC substrates such as Nek2A or cyclin A that are normally degraded in early mitosis. For example, overexpression of cyclin A has been reported to delay chromosome congression (den Elzen, N., and Pines, J. (2001). J Cell Biol 153, 121-136). To test whether stabilization of these substrates is important for the proTAME-induced arrest, HeLa cells were released from double thymidine block into nocodazole for 15 h to allow degradation of cyclin A and other APC substrates that are not efficiently stabilized by the SAC. Cells were then washed out of nocodazole into proTAME. Under this condition, proTAME remained capable of inducing a prolonged mitotic arrest that was highly hesperadin-sensitive (FIG. 21C). This result indicates that the SAC-dependence of proTAME-induced mitotic arrest is unlikely to be caused by stabilization of APC substrates that are normally degraded in a SAC-independent fashion.

Metaphase Arrest Induced by a Proteasome Inhibitor is SAC-Dependent.

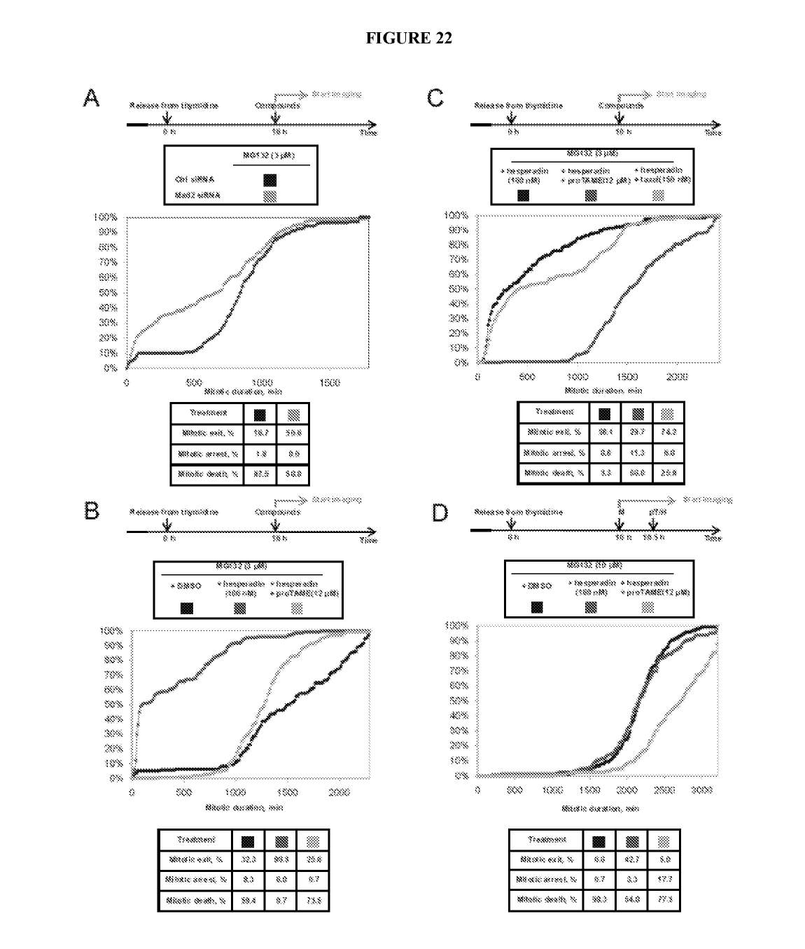

Previous work has indicated that APC-dependent ubiquitination promotes SAC inactivation in cell lysates (Reddy, S. K., et al. (2007). Nature 446, 921-925), potentially explaining why the proTAME arrest is SAC-dependent. In the cell lysate system, APC-dependent ubiquitination of Cdc20, but not APC-dependent proteolysis, was suggested to be important for release of Cdc20 from SAC proteins (Reddy, S. K., et al. (2007). Nature 446, 921-925). However, a recent study found that proteasome activity is required for dissociation of the Mad2-Cdc20 complex (Visconti, R., et al. (2010). Cell Cycle 9, 564-569). Together with our findings, these studies suggested that APC-dependent proteolysis could be important for SAC inactivation. A prediction of this model is that mitotic arrest induced by treatment with a proteasome inhibitor should be SAC-dependent. To test this idea, cells were treated with a low dose of proteasome inhibitor (3 μM) that was sufficient to arrest cells in mitosis (median duration of 15 h). At this concentration, the duration of arrest was limited by cell death rather than mitotic exit, as only 10% of cells exited mitosis during the 30 h duration of the experiment (FIG. 22A). In MG132-treated cells depleted of Mad2 by RNAi, 50% of the cells exited mitosis (FIG. 22A), indicating that the SAC is required to prevent mitotic exit.

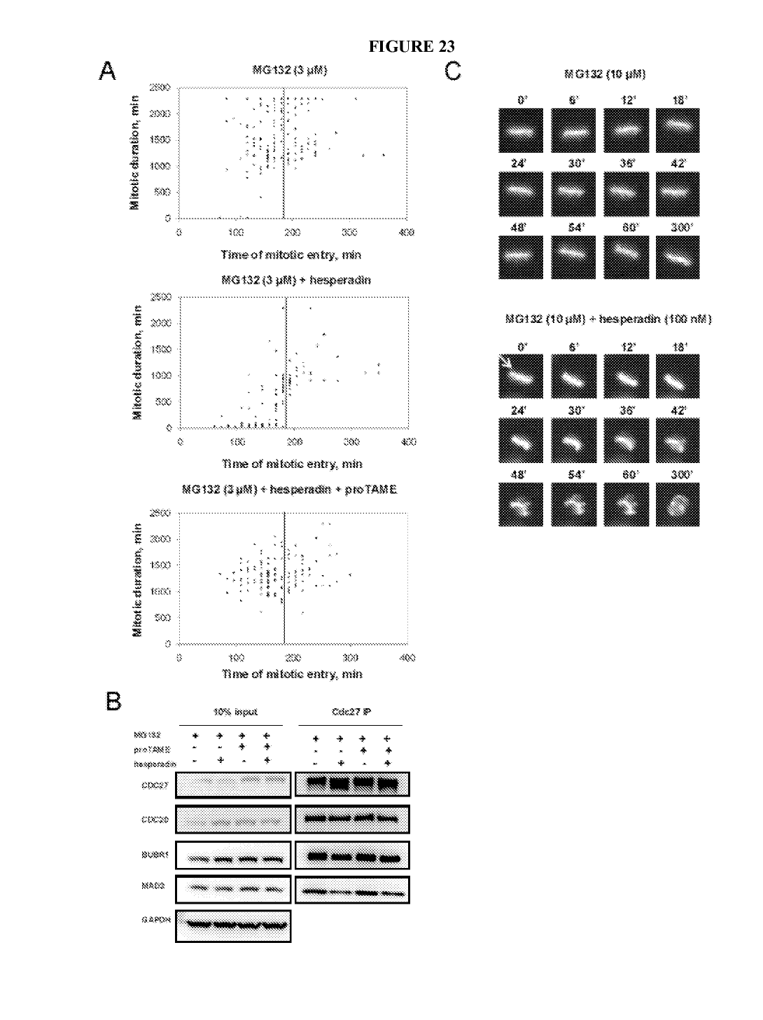

Like proTAME-treated cells, MG132-treated cells also arrest in metaphase with apparently normal kinetochore attachment (Famulski, J. K., and Chan, G. K. (2007). Curr Biol 17, 2143-2149). Given that the MG132-induced arrest is Mad2-dependent, it was next determined whether the arrest is also hesperadin-sensitive. To test this idea, ten hours following thymidine release, HeLa H2B-GFP cells were treated with 3 μM MG132 in the presence or absence of hesperadin. Strikingly, hesperadin induced rapid mitotic exit in half of the cells, with the remainder exiting mitosis more slowly (FIG. 22B). These distinct behaviors correlated with the timing of drug administration: cells that encountered drug while in mitosis exited mitosis quickly, whereas cells that encountered drug before mitosis exited slowly (FIG. 23A). Hesperadin treatment induced dephosphorylation of Cdc27 and reduced levels of Mad2 and BubR1 bound to the APC compared to cells treated with MG132 alone (FIG. 23B). Co-addition of proTAME to MG132 abrogated the ability of hesperadin to drive mitotic exit (FIG. 22B), indicating that mitotic exit under these conditions requires APC-dependent ubiquitination. In contrast, co-addition of taxol to MG132 did not efficiently suppress hesperadin-induced mitotic exit (FIG. 22C), underscoring the distinct mechanisms underlying taxol and proTAME-induced mitotic arrests. Even when the proteasome was more fully inhibited by increasing the MG132 concentration to 10 μM (FIG. 22D), addition of hesperadin caused 40% of cells to exit mitosis (FIG. 22D), implying that sufficient proteasome activity remains in these cells to permit mitotic exit if the SAC is inactivated. Addition of proTAME suppressed the ability of hesperadin to induce mitotic exit, indicating that mitotic exit remains dependent on APC-dependent ubiquitination. Together these results indicate that the mitotic arrest induced by a proteasome inhibitor is not simply a consequence of direct inhibition of the proteasome, but also requires inhibition of APC-dependent ubiquitination by the SAC.

The hesperadin sensitivity of the MG132-induced arrest indicated that Aurora B activity is important for maintaining the metaphase plate, as shown in proTAME treated cells. Consistent with this idea, treatment of MG132-arrested metaphase cells with hesperadin induced deformation of the metaphase plate within 30 min, whereas cells arrested with MG132 alone maintained a normal-appearing metaphase plate for over 5 h (FIG. 23C). These results suggest that an Aurora B-dependent process is required for maintaining the metaphase plate in MG132-treated cells Inhibitors of APC-dependent proteolysis produce a metaphase arrest that is SAC-dependent because Aurora B-dependent pathways remain active in metaphase.

Protein Synthesis is Required for Mitotic Arrest Induced by Microtubule Inhibitors but not for APC or Proteasome Inhibitors.

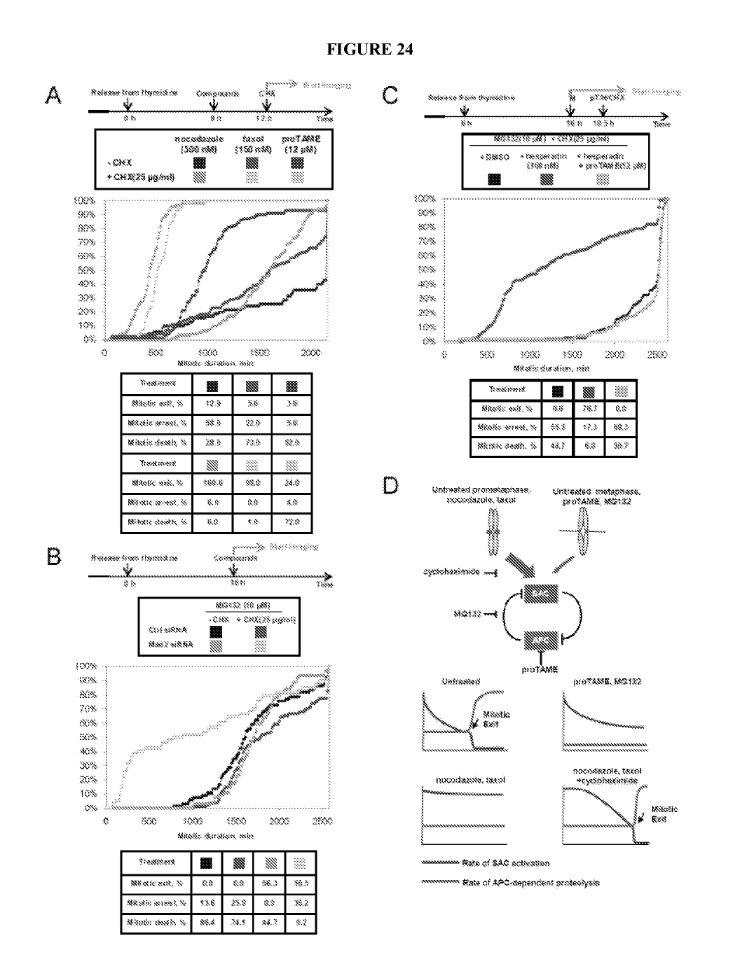

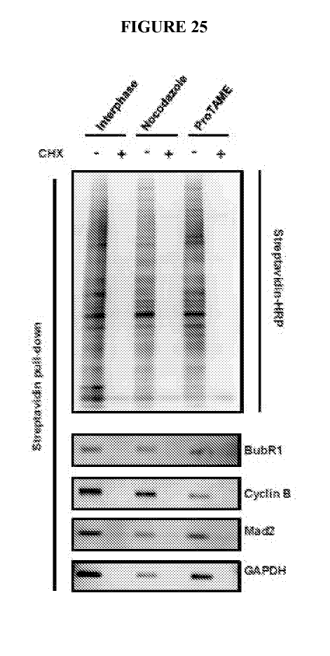

Our data suggest a model in which APC-dependent proteolysis is required to inactivate the SAC. However, this model yields a paradox: How could the APC initiate SAC inactivation if it is fully inhibited by the SAC? One possibility is that SAC inhibition of APC is never complete, with residual APC remaining active to initiate SAC inactivation. Indeed, it has been shown that cyclin B1 and securin are slowly degraded even in the presence of a fully active SAC (Brito, D. A., and Rieder, C. L. (2006). Curr Biol 16, 1194-1200; Nilsson, J., et al. (2008). Nat Cell Biol 10, 1411-1420). Prolonged arrest in mitosis might therefore require the continued synthesis of APC substrates during mitosis. Consistent with this model, cycloheximide promoted mitotic exit of nocodazole- or taxol-arrested cells (FIG. 24A). In striking contrast, cycloheximide did not accelerate mitotic exit in proTAME-treated cells, but rather extended mitotic arrest by delaying cell death (FIG. 24A). Cycloheximide addition produced similar effects in MG132-treated cells, suppressing cell death without promoting mitotic exit (FIG. 24B). Consistent with these findings, labeling experiments demonstrated that known APC substrates such as cyclin B1 and BubR1 are translated during mitotic arrest (FIG. 25). Together these findings indicate that ongoing mitotic protein synthesis is essential to maintain a SAC-dependent mitotic arrest, perhaps by replenishing components that are degraded by residual APC-dependent proteolysis.

Studies were carried out to understand why the MG132-induced arrest is resistant to cycloheximide. It was hypothesized that persistent SAC activity cooperates with direct pharmacologic inhibition of the proteasome to slow the rate of APC-dependent proteolysis to such a great extent that mitotic arrest no longer depends upon protein synthesis. If this hypothesis is correct, then inactivating the SAC should make the MG132-induced arrest sensitive to cycloheximide, as protein synthesis would now be required to balance the increased rate of APC-dependent degradation. This was indeed the case, as depletion of Mad2 (FIG. 24B) or inactivation of the SAC with hesperadin (FIG. 24C) led to mitotic exit in cells treated with cycloheximide and high dose MG132. Addition of proTAME suppressed the effect of hesperadin (FIG. 24C), indicating that mitotic exit remains dependent on APC-mediated ubiquitination. Together these results indicate that the ability of proTAME or proteasome inhibitors to induce a prolonged mitotic arrest independent of protein synthesis requires persistent inhibition of the APC by the SAC.

The mechanism of the first small molecule inhibitor of the APC has been identified. TAME binds to the APC and displaces the IR tail of Cdc20 or Cdh1, preventing efficient APC activation. In human cells, proTAME treatment causes arrest in metaphase without perturbing the mitotic spindle. Despite development of normal kinetochore tension that should silence the SAC, the SAC is required for proTAME to induce mitotic arrest. Similar results were obtained using a proteasome inhibitor. Kinetochore-dependent SAC signaling persists at a low rate in metaphase, and is inactivated by residual APC-dependent proteolysis, creating a positive feedback loop between the APC and the SAC (FIG. 8D). The ability of low doses of proTAME or MG132 to induce metaphase arrest is strongly enhanced by this feedback loop, enabling mitotic arrest to be achieved at drug concentrations below those necessary to fully inhibit the APC or the proteasome.

TAME Interferes with IR-Tail Dependent APC Activation.

TAME prevents APC activation by perturbing the binding of the IR-tail of Cdc20 and Cdh1 to the APC. The importance of the IR motif in promoting Cdh1 association with yeast and human APC is well-established (Burton, J. L., et al. (2005). Mol Cell 18, 533-542; Kraft, C., et al. (2005). Mol Cell 18, 543-553; Matyskiela, M. E. and Morgan, D. O. (2009). Mol Cell 34, 68-80; Vodermaier, H. C., et al. (2003). Curr Biol 13, 1459-1468). However, the role of the Cdc20 IR motif is less clear, because the Cdc20 IR tail is not essential in budding yeast (Thornton, B. R., et al. (2006). Genes Dev 20, 449-460) and a Cdc20ΔIR mutant can support APC-dependent degradation of Nek2A in Xenopus extract (Kimata, Y., et al. (2008). Mol Cell 32, 576-583). Our data show that the IR motif of Cdc20 indeed contributes significantly to APC-association in vitro, as Cdc20ΔIR binds the APC with lower affinity than the wild type protein, and TAME competes with wild type Cdc20 for APC association. Moreover, TAME induces significant dissociation of Cdc20 from the APC in Xenopus extract, and proTAME can antagonize Cdc20 binding in human cells if the SAC is inactivated. Functionally, TAME stabilizes APC substrates in Xenopus extract and proTAME inhibits both Cdc20 and Cdh1-dependent degradation in HeLa cells. Taken together, these data show that proper engagement of the IR motif of Cdc20 or Cdh1 is critical for APC activation.

TAME Exploits a Positive Feedback Loop Between the SAC and the APC.

ProTAME-induced mitotic arrest requires sustained SAC activity. This finding was unexpected, because proTAME-treated cells arrest in metaphase with kinetochores that develop normal tension, a condition that should inactivate the SAC. In principle, the requirement for the SAC in the proTAME arrest could be explained in one of two ways. First, proTAME treatment could produce defects in microtubule-kinetochore interactions that generate an abnormally high degree of checkpoint signal compared to normal metaphase kinetochores. Alternatively, proTAME may hamper SAC inactivation, despite normal microtubule-kinetochore interactions. The latter model is favored because the degree of checkpoint dependence far exceeds the observed degree of kinetochore-microtubule perturbation.

Defects in microtubule-kinetochore attachment are either an off-target effect of TAME on microtubules or a consequence of specific APC inhibition. Knockdown of Cdc27 or UbCH10 each produced a mitotic exit delay that was SAC-dependent. Furthermore, treatment of cells with a proteasome inhibitor yielded a SAC-dependent mitotic arrest, consistent with a recent study showing that MG132-treated mitotic cells show persistent Mad2-Cdc20 interaction (Visconti, R., et al. (2010). Cell Cycle 9, 564-569), and work in S. pombe showing that Mad2 and Mad3 remain APC-bound in proteasome mutants (Ohi, M. D., et al., (2007). Mol Cell 28, 871-885). Together these findings indicate that if defective microtubule kinetochore interactions are indeed present in proTAME-treated cells, they are likely to result from specific inhibition of APC-dependent proteolysis rather than from nonspecific effects of proTAME on microtubules.

If defective microtubule-kinetochore interactions exist in proTAME-treated cells, they must be subtle. Cells treated with 12 μM proTAME arrest in mitosis until they die, yet form a normal-appearing metaphase plate and develop normal kinetochore tension. Furthermore, cells treated with 12 μM proTAME undergo a normal-appearing anaphase when the SAC is inactivated, indicating that the mitotic spindle functions properly in the presence of proTAME. The only change in chromosome behavior caused by this dose of proTAME is a slight delay in chromosome congression. A lower dose of proTAME (3 μM) causes no delay in chromosome congression, yet still extends mitotic duration to 5 hours. Although subtle defects in microtubule-kinetochore interactions in proTAME-treated cells cannot be completely ruled-out, such defects are not of sufficient magnitude to explain the strong dependence of the proTAME arrest on the SAC.

The alternative explanation for the SAC-dependence of the proTAME arrest is that APC-dependent ubiquitination or proteolysis is required to inactivate the SAC. Such mutual antagonism between the APC and the SAC is predicted to create a positive feedback loop that would amplify the inhibitory effects of proTAME or a proteasome inhibitor in a SAC-dependent manner. This is what occurred. If the SAC is inactivated by Mad2 depletion, 12 μM proTAME extends mitotic duration by only 72 minutes, indicating that this dose only partially inhibits APC activation (consistent with the measured IC₅₀ of 12 μM in Xenopus extract). However, when the same dose of proTAME is used in cells with an intact SAC, proTAME extends mitotic duration by 23 hours, indicating that the effect of proTAME is greatly amplified by the SAC. This degree of amplification cannot be explained by the mild effect of proTAME on chromosome congression, because a dose of nocodazole (10 nM) that causes a similar delay in chromosome congression extends mitotic duration by only 20 minutes in SAC-proficient cells. Because similar results were obtained with a proteasome inhibitor, this amplification is best explained by a requirement for APC-dependent proteolysis to inactivate the SAC.

APC substrates play an important role in mediating the mutual antagonism between the APC and the SAC. APC-dependent ubiquitination of Cdc20 has been proposed to release the APC from the inhibitory effects of the SAC (Reddy, S. K., et al. (2007). Nature 446, 921-925; Stegmeier, F., et al. (2007). Nature 446, 876-881). However, this process does not require proteasome activity in cell lysates (Reddy, S. K., et al. (2007). Nature 446, 921-925), and others argue that Cdc20 ubiquitination targets Cdc20 for proteasomal degradation in a manner that sustains the SAC (Ge, S., et al. (2009). Cell Cycle 8, 167-171; Nilsson, J., et al. (2008). Nat Cell Biol 10, 1411-1420). Alternatively, many SAC proteins are APC substrates, and may need to be degraded to inactivate the SAC. Consistent with this possibility, expression of a stable BubR1 mutant induces a mitotic arrest (Choi, E., et al. (2009). EMBO J. 28, 2077-2089). Another candidate is cyclin B, because it is degraded prior to anaphase (Clute, P., and Pines, J. (1999). Nat Cell Biol 1, 82-87) and cyclin-dependent kinase activity is required to maintain the SAC (Chung, E. and Chen, R. H. (2003). Nat Cell Biol 5, 748-753; D'Angiolella, V., et al. (2003). Genes Dev 17, 2520-2525). Other SAC proteins, including Mps1, Bub1, and Aurora B are also APC substrates, but their bulk population is not degraded until after anaphase (Palframan, W. J., et al. (2006). Science 313, 680-684; Qi, W. and Yu, H. (2007). J Biol Chem 282, 3672-3679; Stewart, S. and Fang, G. (2005). Cancer Res 65, 8730-8735). It is possible that degradation of these proteins prior to anaphase is masked by their resynthesis. The mutual antagonism between the APC and the SAC may reflect a system-level behavior that is regulated by small changes in the abundance of multiple SAC proteins prior to anaphase. If so, confirmation of our model will require quantitative measurements of the relative rates of synthesis and degradation of APC substrates that regulate SAC activity.

Our results indicate that it is possible to induce mitotic arrest without fully inhibiting the APC or the proteasome pharmacologically. This result was unexpected, because RNAi-based experiments indicated that Cdc20 must be reduced to very low levels to induce mitotic arrest (Wolthuis, R., et al. (2008). Mol Cell 30, 290-302). Unlike the proTAME-induced arrest, the mitotic arrest induced by Cdc20 knockdown does not depend on the SAC (Huang, H. C., et al. (2009). Cancer Cell 16, 347-358). One possible explanation for the lack of SAC-dependence in the context of Cdc20 depletion is that Cdc20 is the target of the SAC (Yu, H. (2007). Mol Cell 27, 3-16). Therefore, when Cdc20 levels are reduced, the SAC is no longer required to inhibit Cdc20 function. In contrast, other methods of perturbing APC function, including knockdown of core APC subunits or the E2 enzyme UbCH10, or proTAME treatment, all produce an arrest that is SAC-dependent. This is likely a consequence of the fact that Cdc20 remains present under each of these conditions.

A Model for Regulation of Mitotic Exit.

A model is shown in FIG. 24D. A positive feedback loop between the SAC and the APC has the potential to adopt one of two stable states: high SAC activity (mitotic arrest) or high APC activity (mitotic exit). During normal division, it is important that cells do not become permanently arrested in mitosis. The SAC does not fully inhibit the APC during mitosis because residual APC activity must be preserved to prevent cells from becoming locked in mitosis. This residual APC activity may explain why cyclin B1 is degraded prior to the initiation of anaphase (Clute, P., and Pines, J. (1999). Nat Cell Biol 1, 82-87) and during prolonged SAC-dependent mitotic arrest (Brito, D. A. and Rieder, C. L. (2006). Curr Biol 16, 1194-1200; Gascoigne, K. E. and Taylor, S. S. (2008). Cancer Cell 14, 111-122; Huang, H. C., et al. (2009). Cancer Cell 16, 347-358; Huang, H. C., et al. (2009). Cancer Cell 16, 347-358; Nilsson, J., et al. (2008). Nat Cell Biol 10, 1411-1420). To remain in mitosis for a prolonged period, a cell may need to continue to resynthesize APC substrates that are degraded by residual APC-dependent proteolysis.

During normal mitosis, the development of kinetochore tension reduces the rate of SAC activation, but SAC activation is unlikely to be completely suppressed during metaphase. Anaphase is triggered when the rate of SAC activation falls below the rate at which APC-dependent proteolysis inactivates the SAC, tipping the feedback loop toward rapid APC activation and mitotic exit. The timing of anaphase initiation therefore depends not only on how kinetochore attachment controls SAC activation, but also on the level of residual APC activity.

During nocodazole or taxol treatment, the rate of SAC activation remains above the rate at which the APC inactivates the SAC, tipping the loop in the direction of APC inhibition thereby preventing mitotic exit. Because APC-dependent proteolysis is not fully inhibited by the SAC, mitotic arrest is dependent on protein synthesis to resupply APC substrates. If the rate of protein synthesis is not sufficient, the rate of SAC signal production will fall below the rate at which it is inactivated by the APC, leading to rapid APC activation and mitotic slippage. Therefore, the rate of protein synthesis in mitosis may be an important determinant of the duration of mitotic arrest in cells treated with microtubule inhibitors.

In contrast to microtubule inhibitors, proTAME and MG132 induce mitotic arrest by inhibiting residual APC-dependent proteolysis rather than by stimulating SAC activation. The rate of SAC signal production by kinetochores may decline normally in proTAME- or MG132-treated cells because kinetochores develop proper tension. However, because the rate of residual APC-dependent proteolysis is lowered by proTAME or MG132, the rate of SAC signal production cannot fall below the rate at which it is inactivated by APC-dependent proteolysis, leading to mitotic arrest. The strong hesperadin sensitivity of both proTAME and MG132-induced arrests indicates the importance of metaphase kinetochores in generating a SAC signal to sustain mitotic arrest. Compared to microtubule inhibitors, this mechanism of mitotic arrest shows reduced dependence on protein synthesis because the rate of residual APC activity is lower in proTAME and MG132-treated cells, yielding a lower requirement for protein synthesis to replenish APC substrates.

Antimitotic Cancer Therapy.

Our study has identified an explanation for the variability in cellular responses to microtubule inhibitors that could limit their therapeutic effectiveness. Because the SAC does not completely inhibit the APC, mitotic arrest induced by microtubule inhibition depends on protein synthesis. As a result, variation in the rates of protein synthesis among cells may be one factor that explains the highly variable response of cells to microtubule inhibitors. In contrast, cells treated with an APC inhibitor are less prone to mitotic slippage because residual APC activity is inhibited. APC inhibitors may therefore be more effective in promoting mitotic arrest, inducing a greater pro-apoptotic effect. Furthermore, low doses of an APC inhibitor are useful in combination with microtubule inhibitors to sustain mitotic arrest and enhance cell death.

### Experimental Procedures

### Chemicals and Antibodies

Chemicals.

Tosyl-L-arginine methyl ester (T4626), tosyl-L-arginine (S365157), tosyl-L-argininamide (T4501), benzoyl-L-arginine methyl ester (B1007), benzoyl-L-argininamide (B4375) and tosyl-L-lysine methyl ester (T5012) were from Sigma. Acetyl-L-arginine methyl ester was from BACHEM (E-1030). Cdh1 C-terminal peptide and the ΔIR control peptide (sequence: CFSKTRSTKESVSVLNLFTRIR (SEQ ID NO: 2) and CFSKTRSTKESVSVLNLFTR (SEQ ID NO: 3)) were synthesized by the core facility of Tufts medical school. ³H-TAME (15 Ci/mmol, >97% radiochemical purity) was synthesized by AmBios Labs (Newington, Conn.). Hesperadin was a gift from Boehringer Ingelheim. MG132 was from Sigma (C2211). Okadaic acid was from MP Biomedicals (IC15897425). Cycloheximide was from Calbiochem (239764).

Antibodies.

Cdc27 antibody for APC immunoprecipitation was from Santa Cruz (sc-9972, AF3.1). Cdc27 antibody for Western blot was from BD Transduction Laboratories (610454). Cyclin B1 antibody was from NeoMarker (RB-008-P). Xenopus Cdc20 antibody was from Abcam (ab18217). Human Cdc20 antibody was from Santa Cruz (sc-8358 H-175). Cdh1 antibody was from Santa Cruz (sc-19398). Streptavidin-HRP was from Invitrogen (SNN1004). Securin antibody was from Abcam (ab3305). Cyclin A antibody was from Santa Cruz (sc H-432). Nek2 antibody was from BD Transduction Laboratories (610593). UbcH10 antibody was from Boston Biochem (A-650). Mad2 antibody was from Bethyl Laboratories (BL1461). GAPDH antibody was from Abcam (ab8245). Anti-α-tubulin-FITC was from Sigma (F2168). CREST antiserum was from Antibodies Incorporated (15-234). Goat anti-human-Alexa 568 was from Invitrogen (A21090). HA antibody was from Santa Cruz (sc-805, Y-11). Apc10 antibody was from Santa Cruz (sc-20989).

### Preparation of Xenopus Egg Extract.

Interphase Xenopus egg extract was prepared from eggs laid overnight according to the protocol of Murray (Murray, A. W. (1991). Chapter 30 Cell Cycle Extracts. Methods Cell Biol 36, 581) with the exception that eggs were activated with 2 μg/ml calcium ionophore (A23187, free acid form, Calbiochem) for 30 minutes prior to the crushing spin. Extract was frozen in liquid nitrogen and stored at −80° C. To make mitotic extract, MBP-cyclin B1 Δ90 was added to interphase extract at 20 μg/ml and incubated at 22° C. for 30 min.

### Luciferase Assay.

A fusion of the N-terminal domain of cyclin B1 to luciferase (Verma, R., et al., (2004). Science 306, 117-120) was added to mitotic extract at 3 μg/ml. The extract was incubated at 23° C. and 3 μl samples were taken at 0, 30, 60, 90 and 120 min. The samples were mixed quickly with 30 μl of luciferin assay buffer (270 μM coenzyme A, 20 mM tricine, 3.67 mM MgSO₄, 0.1 mM EDTA, 33.3 mM DTT, 530 μM ATP and 470 μM luciferin, pH 7.8) and the level of luminescence was measured on Wallac 1420 multilabel counter.

TAME induction of mitotic arrest in Xenopus egg extract.

Human cyclin B1/cdc2 complex (MPF) was prepared by baculovirus expression and purification as described Kirkpatrick, D. S., et al. ((2006). Nat Cell Biol 8, 700-710), and added to interphase extract at 12.5 μg/ml supplemented with 1% DMSO, 200 μM TAME or 200 μM AAME. Extract samples were collected every 15 min following addition of MPF. Cdc27 and cyclin B1 levels were analyzed by Western blot.

### In Vitro Ubiquitination Assay.

For a single reaction, 5 μl protein A affiprep beads (Bio-Rad 156-0006) were washed with TBST (10 mM Tris, 150 mM NaCl and 0.01% Tween-20, pH 7.5) twice and incubated with 2 μg Cdc27 antibody for 75 min at 4° C. Beads were then washed with TBST twice and XB (100 mM KCl, 0.1 mM CaCl₂, 1 mM MgCl₂ and 10 mM HEPES, pH 7.7) twice before APC immunoprecipitation. For APC-Cdc20 reaction, MBP-cyclin B1490 was added to interphase extract at 20 μg/ml and incubated at 22° C. for 30 min before immunoprecipitation. To immunoprecipitate APC, 100 μl extract was incubated with 5 μl antibody beads at 4° C. for 1 h. The beads were then washed with XB high salt (XB with 500 mM KCl) twice, XB twice and ubiquitin chain buffer (20 mM Tris, 100 mM KCl, 2 mM ATP and 2.5 mM MgCl₂, pH 7.7) three times. A reaction mixture containing 200 μg/ml MBP-E1, 66 μg/ml His₆-Ubc4, 25 μg/ml MPF, 1 mg/ml ubiquitin (Sigma) in ubiquitin chain buffer was prepared and 5 μl of this was added to 5 μl antibody beads. Beads were incubated at 22° C. on a Eppendorf Thermomixer with shaking at 1500 rpm for 60 min and the whole mixture was then boiled with 10 μl sample buffer for 5 min. Ubiquitinated cyclin B1 was visualized by cyclin B1 immunoblot.

### Degradation of ³⁵S Labeled Pre-Ubiquitinated Cyclin B1.

Human ³⁵S-cyclin B1/cdc2 complex was prepared by metabolic labeling of SF9 cultures expressing cyclin B1. The labeled lysate containing cyclin B1 was mixed with an unlabeled lysate from cells expressing cdc2, followed by purification of the cyclin B1/cdc2 complex as described above. The labeled complex was ubiquitinated in a reconstituted APC reaction as described above. Interphase Xenopus extract was pre-incubated with 200 μM TAME or control compounds for 22° C. for 30 min. Pre-incubation was performed in the presence of 100 ug/ml cycloheximide to prevent re-incorporation of free labeled amino acid and 1/20th volume of energy mix (150 mM creatine phosphate, 20 mM ATP, 2 mM EGTA and 20 mM MgCl₂, pH 7.7). 90 μl of extract was then added to 15 μl of labeled cyclin B1-ubiquitin conjugates and incubated at 22° C. for the indicated amount of time. Reactions were stopped by the addition of an equal volume (105 ul) of chilled 2% perchloric acid. The mixture was incubated on ice for 30 min and centrifuged at 14,000 rpm for 10 min at 4° C. 168 μl of supernatant was mixed with 20 μl 2 M Tris base and 6 ml ultima gold scintillation fluid (Perkin Elmer). Samples were mixed well and counted with a scintillation counter.

### Covalent Coupling of Cdc27 Antibody to Protein A Beads.

Protein A affiprep beads were coupled with Cdc27 antibody as described above. After coupling, the beads were washed with TBST for 10 min followed by two additional quick washes with TBST. Dimethyl pimelimidate (DMP, PIERCE, 21666) was freshly dissolved in 100 mM sodium tetraborate decahydrate, pH 9.0 at 20 mM. The beads were mixed with ten beads volume of DMP solution and incubated on a rotating wheel for 45 min in the dark at room temperature. The beads were then washed twice quickly with 200 mM Tris, pH 8.0 twice, followed by a final 1 h wash. Beads were then washed twice in TBST and twice in XB prior to APC immunoprecipitation.

### IR Peptide Immobilization on Iodoacetyl Resin.

A 20-aa Cdh1 C-terminal peptide with one cysteine residue added at the N-terminus was synthesized along with a control peptide lacking the C-terminal IR residues. The lyophilized peptide was re-dissolved at 400 μM in 100 mM HEPES, 5 mM EDTA, pH 7.9. To reduce the disulfide bonds, TCEP (Sigma C4706) was dissolved at 10 mM in 100 mM HEPES, 5 mM EDTA, pH 7.9 and added to the peptide solution at a final concentration of 200 μM (stoichiometric amount to reduce disulfide bonds). The peptide was reduced at room temperature for 15 min before mixing with Ultralink iodoacetyl resin (Pierce 53155) that was pre-equilibrated with 100 mM HEPES, 5 mM EDTA, pH 7.9. A ratio of 35 μl resin volume per 110 μl reduced peptide was used. For the negative control, freshly prepared 50 mM cysteine in 100 mM HEPES, 5 mM EDTA, pH 7.9 was used instead of the peptide. The coupling reaction was carried out at room temperature on a rotating wheel for 1 h and unreacted sites on the resin were blocked by further incubation with 50 mM cysteine for 30 min. The resin was then washed with 1 M NaCl followed by two washes with XB and stored at 4° C. before APC pull down.

APC pull down by IR peptide resin. 10 μl of resin coupled with IR peptide, ΔIR peptide or cysteine as described above was mixed with 100 μl interphase Xenopus egg extract and incubated on a rotating wheel for 30 min at 4° C. The resin was then washed twice with XB high salt and once with PBS. The resin was boiled with 10 μl sample buffer and the amount of Cdc27 was analyzed by immunoblot.

Conjugation of IR Peptide with Photoactive Crosslinker.

The IR and ΔIR peptides were reduced as described above. The photoactive crosslinker, Profound Mts-Atf-Biotin label transfer reagent (Pierce 33093), was dissolved at 40 mg/ml in DMSO. The crosslinker was added to the reduced peptide at a 1.1 molar excess and the reaction was left at room temperature in the dark for 1 h. The reaction mixture was centrifuged at 12,000 rpm for 1 min and the supernatant was loaded onto HPLC for purification. The purified conjugated peptide showed >99% purity on HPLC. The identity of the conjugated peptide was confirmed by mass spectrometry.

### Crosslinking Assay.

Purified conjugated IR or ΔIR peptide was diluted in XB to a final concentration of approximately 2 μM. The following additives were included when necessary: 10 μM of unconjugated IR peptide with the cysteine modified with N-ethyl maleimide to show competition with the conjugated peptide, 20 μM or 200 μM TAME to show inhibition of crosslinking, and 200 μM AAME as a negative control. APC was immunoprecipitated with protein A affiprep beads covalently crosslinked with Cdc27 antibody as described above. After washing, 5 μl aliquot of beads were mixed with 50 μl conjugated peptide and transferred to a 96-well polypropylene clear conical bottom plate. The plate was illuminated at a distance of 10 cm from a 300 watt long wavelength UV lamp for 3 min. The beads were then transferred back to 0.5 ml tubes and mixed with 10 μl sample buffer and boiled for 5 min. APC subunits that were crosslinked were analyzed by streptavidin-HRP blot. To confirm the nature of the crosslinked subunits, APC was immunoprecipitated from interphase extract as described above and run on the same gel of the crosslinked sample and coomassie stained. The bands were subjected to mass spectrometry analysis.

### ³H-TAME Binding Assay.

³H-TAME (200 nM; 15 Ci/mmol) was added to 100 μl interphase Xenopus extract or HeLa cell lysate. APC was immunoprecipitated with Cdc27 antibody (Santa Cruz, AF3.1) coupled to affiprep beads (Bio-Rad) as previously described (Kirkpatrick, D. S., et al. (2006). Nat Cell Biol 8, 700-710). The beads were washed with XB and radioactivity measured by scintillation counting. Alternatively, ³H-TAME was added and Cdc27 immunoprecipitation was performed after one or two rounds of APC immunodepletion. Specific binding was calculated as the difference between counts associated with Cdc27 antibody beads compared to beads lacking antibody (mock IP).

### ³H-TAME Binding Assay in Interphase Extract.

³H-TAME (15 Ci/mmol) was added to interphase extract (100 μl) to a final concentration of 200 nM, and subject to immunoprecipitation (4° C. for 1.5 h) using 5 μl protein A affiprep beads coupled with Cdc27 antibody as described above. Protein A beads without Cdc27 antibody were used as a negative control to measure background level of binding (mock IP). The beads were washed quickly twice with XB high salt and twice with XB. The beads were then transferred to scintillation vials, mixed with scintillation fluid, and counted in a scintillation counter. Alternatively, the extract was subjected to one or two rounds of immunoprecipitation before the addition of ³H-TAME. For competition assays, different concentrations of unlabeled TAME or other derivatives were added along with 200 nM ³H-TAME into the extract. Specific binding under each condition was obtained by subtracting the value of mock IP.

### ³H-TAME Binding Assay in HeLa Cell Lysate.

Protein A affiprep beads coupled with Cdc27 antibody were prepared as described above. HeLa cells were harvested in lysis buffer (10 mM potassium phosphate pH 7.5, 0.1 mM EDTA, 0.5 mM EGTA, 50 mM (3-glycerophosphate, 1 mM sodium vanadate, 1 mM DTT, 0.5% Triton X-100 and leupeptin, chymostatin and pepstatin each at 10 μg/ml). For 4,000,000 cells, 100 μl lysis buffer was used. The cell lysate was centrifuged at 10,000 rpm for 10 min to remove cell debris. ³H-TAME (15 Ci/mmol) was added to cell lysate to a final concentration of 200 nM. For each aliquot of 5 μl beads, 100 μl lysate was used for APC immunoprecipitation at 4° C. for 1 h. Protein A beads without Cdc27 antibody was used as a negative control to measure background level of binding (mock IP). The beads were washed quickly with lysis buffer high salt (500 mM sodium chloride in addition to above components) twice and lysis buffer twice. The beads were then transferred to scintillation vials, mixed with scintillation fluid, and radioactivity measured by scintillation counting. Alternatively, the lysate was subjected to one or two rounds of immunoprecipitation before the addition of ³H TAME. For competition assays, 10 μM unlabeled TAME or AAME was added along with 200 nM ³H TAME into the lysate. Specific binding under each condition was obtained by subtracting the value of mock IP.

### APC Isolation by IR Peptide or C-Box Fragment and Crosslinking.

A cysteine-containing 20 amino acid peptide derived from the C-terminus of Cdh1, or a control peptide lacking the C-terminal isoleucine and arginine residues, was reduced with TCEP at RT for 15 min and coupled to Ultralink iodoacetyl resin (Pierce). Ten μl of resin was mixed with 100 μl interphase Xenopus egg extract and incubated on a rotator for 30 min at 4° C. The resin was then washed with XB (100 mM KCl, 0.1 mM CaCl₂, 1 mM MgCl₂ and 10 mM HEPES, pH 7.7) and bound Cdc27 was analyzed by immunoblot. To investigate the effect of TAME on C-box interactions, a GST fusion protein containing the N-terminal 159 residues of Xenopus Cdc20, or the same protein lacking the C-box, were expressed and purified as described previously (Kimata, Y., et al. (2008). Mol Cell 32, 576-583). The proteins (10 μg) were preloaded on 5 μl Glutathione-Sepharose 4B resin (GE Healthcare) and incubated with cyclin B1490-arrested mitotic Xenopus extract at RT for 30 min in the presence of 1% DMSO, 200 μM TAME or 200 μM AAME. The resin was then washed with XB and bound Cdc27 was analyzed by immunoblot. For crosslinking studies, the Cdh1-derived C-terminal peptide was conjugated to Profound Mts-Atf-Biotin label transfer reagent (Pierce) and crosslinked as described in the supplemental experimental procedures.

### IR Peptide Crosslinking Assay.

The Cdh1-derived C-terminal peptide was conjugated to Profound Mts-Atf-Biotin label transfer reagent (Pierce) as described in the supplemental experimental procedures. APC was immunoprecipitated from interphase extract and the beads (5 μl) were mixed with 50 μl conjugated peptide (2 μM) and transferred to a 96-well polypropylene plate. The plate was illuminated at a distance of 10 cm from a 300 watt long wavelength UV lamp for 3 min. Results were analyzed by streptavidin-HRP blot. Immunopurified APC was run in parallel, and coomassie stained APC subunits were identified by analyzing co-migrating bands by mass spectrometry.

### In Vitro Ubiquitination Assay.

Human cyclin B1/cdc2 complex was purified and used as the substrate with Ubc4 as the E2 enzyme as previously described (Kirkpatrick, D. S., et al. (2006). Nat Cell Biol 8, 700-710).

### APC-Cdc20/Cdh1 Association Assay.

APC was immunoprecipitated from cyclin B1Δ90-arrested mitotic Xenopus extract or interphase extract supplemented with 0.5 μg/ml recombinant Cdh1 as previously described (Kirkpatrick, D. S., et al. (2006). Nat Cell Biol 8, 700-710). Compounds were added to mitotic extract immediately before immunoprecipitating the APC. Interphase extracts were pre-incubated with compounds for 30 min before adding recombinant Cdh1 and immunoprecipitating the APC. The beads were washed with XB high salt (XB with 500 mM KCl) and then XB, and bound Cdc27 and Cdc20/Cdh1 were analyzed by immunoblot. Alternatively, Cdc20 was expressed using an in vitro coupled transcription/translation reticulocyte lysate system following the manufacturer's instruction (Promega L1170). The lysate was diluted with XB so that the concentration of Cdc20 was approximately equal to that of the endogenous Cdc20 in Xenopus extract. APC was immunoprecipitated from mitotic extract as described above and the beads were washed with XB high salt and XB. For each binding assay, 5 μl beads were mixed with 50 μl diluted lysate plus 1 μM okadaic acid, 0.05% IPEGAL CA-630 and various competitors as indicated for 30 min with constant shaking. The beads were then washed with XB+0.05% IPEGAL CA-630 and bound Cdc27 and Cdc20 were analyzed by immunoblot.

### Live Cell Imaging.

Detailed siRNA transfection and drug treatment schemes are described in supplemental experimental procedures. HeLa H2B-GFP cells were plated in DMEM with 10% FBS at 20% confluence. One day later, cells were synchronized by treatment with 2 mM thymidine for 18 hours, released for 8 hours, and retreated with thymidine for 18 hours prior to release. Live cell imaging was performed at 12 minute intervals on a Nikon TE2000E PFS inverted microscope fitted with an incubation chamber maintained at 37° C. and supplied with 5% CO₂. Cell division was tracked by manual inspection of movies, and mitotic duration was measured as the time between the first frame of chromosome condensation and the frame of chromosome segregation (anaphase), decondensation (mitotic exit without anaphase) or cell death (chromosomes shrinking to a small bright dot).

Synthesis of proTAME (14) and proAAME (15)

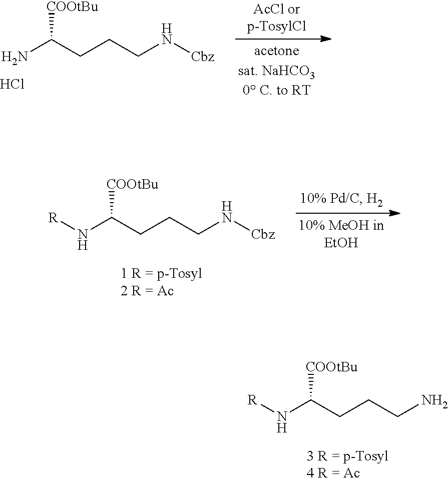

### N²-[(4-methylphenyl)sulfonyl]-N⁵-[(phenylmethoxy)carbony]-L-ornithine 1,2,-dimethylethyl ester (1)

A mixture of N⁵-[(phenylmethoxy)carbonyl]-L-ornithine 1,2,-dimethylethyl ester HCl (718 mg, 2 mmol), acetone (15 mL) and sat. aq. NaHCO₃ (15 mL) was treated with p-toluenesulfonyl chloride (420 mg, 2.2 mmole) in acetone (15 mL) at 0° C. and then stirred at room temperature for 16 h. The mixture was diluted with EtOAc, washed with brine, dried over anhydrous sodium sulfate, filtered, concentrated in vacuo to afford an oil, which was purified by silica gel column chromatography using 40% EtOAc in hexane to give compound 1 (910 mg, 95%): ¹H NMR (500 MHz, CDCl₃): δ1.23 (s, 9H), 1.56-1.65 (m, 3H), 1.73-1.77 (m, 1H), 2.39 (s, 3H), 3.21 (q, J=6.0, 2H), 3.72-3.77 (m, 1H), 4.77 (br, 1H), 5.10 (s, 2H), 5.16 (br, 1H), 7.26-7.28 (m, 2H), 7.31-7.36 (m, 5H), 7.70-7.72 (m, 2H).

### N²-acetyl-N⁵-[(phenylmethoxy)carbony]-L-ornithine 1,2,-dimethylethyl ester (2)

was prepared in a manner similar as 1, with acetyl chloride used in place of p-toluenesulfonyl chloride. ¹H NMR (500 MHz, CDCl₃): δ1.46 (s, 9H), 1.49-1.57 (m, 2H), 1.63-1.69 (m, 1H), 1.81-1.88 (m, 1H), 2.01 (s, 3H), 3.22 (q, J=6.5, 2H), 4.47-4.51 (m, 1H), 4.94 (br, 1H), 5.09 (s, 2H), 6.11 (br d, J=7.5, 1H), 7.30-7.36 (m, 5H).

### N²-[(4-methylphenyl)sulfonyl]-L-ornithine 1,2,-dimethylethyl ester (3)

A mixture of 1 (500 mg, 1.05 mmole), methanol (1.5 mL), ethanol (15 mL) and 10% Pd—C (200 mg) was stirred under a hydrogen atmosphere at room temperature for 3 h. The mixture was filtered through a pad of Celite, concentrated in vacuo to give crude compound 3 (399 mg, 99%). This material was stored in the freezer and then used without further purification.

### N²-acetyl-L-ornithine 1,2,-dimethylethyl ester (4)

was prepared in a manner similar as 3.

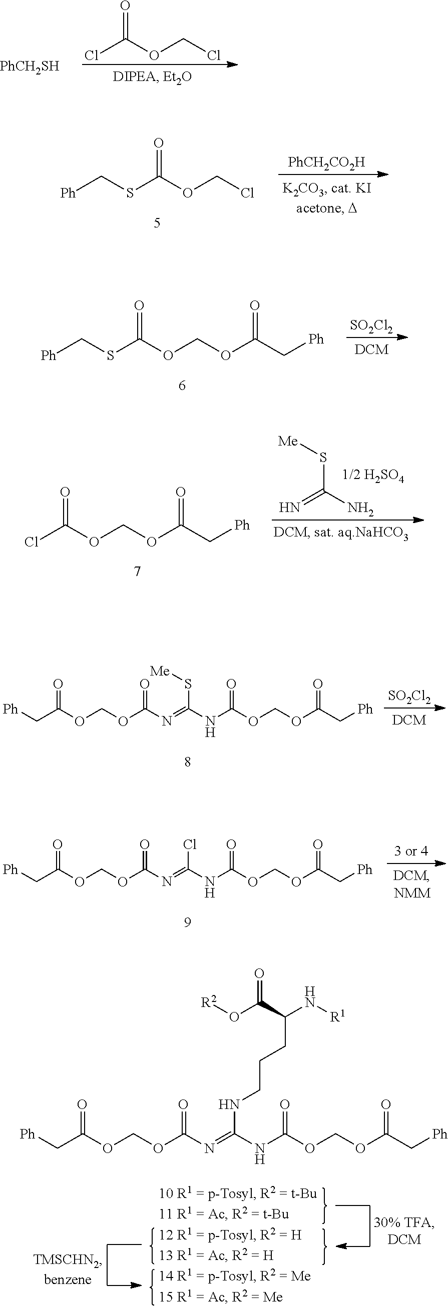

### O-Chloromethyl S-(phenylmethyl)carbothioate (5)

was prepared following a literature procedure (Folkmann, M. L., F. J. (1990). Synthesis, 1159-1166).

### [[(phenylmethylthio)carbonyl]oxy]methyl benzeneacetate (6)

A mixture of 5 (6.327 g, 29.2 mmol), phenylacetic acid (3.68 g, 27 mmole), K₂CO₃ (3.74 g, 27 mmole), and cat. KI in acetone (100 mL) was refluxed for 16 h. The mixture was diluted with EtOAc, washed with brine, dried over anhydrous sodium sulfate, filtered, concentrated in vacuo to afford an oil, which was purified by silica gel column chromatography with 5% EtOAc in hexane to give 1 (5.80 g, 92%): ¹H NMR (500 MHz, CDCl₃): δ 3.68 (s, 4H), 4.12 (s, 4H), 5.82 (s, 4H), 7.26-7.33 (m, 10H).

### [(chlorocarbonyl)oxy]methyl benzeneacetate (7)

was prepared following a literature procedure (Folkmann, M. L., F. J. (1990). Synthesis, 1159-1166).

### 3-(methylthio)-5,9-dioxo-10-phenyl[(phenylacetyl)oxy]methyl 6,8-dioxa-2,4-diazadec-2-enoate (8)

was prepared following a literature procedure (Saulnier, et al. (1994). Bioorg Med Chem Lett 4, 1985-1990). ¹H NMR (500 MHz, CDCl₃): δ 2.44 (s, 3H), 3.70 (s, 4H), 5.84 (s, 4H), 7.26-7.35 (m, 10H).

### 3-chloro-5,9-dioxo-10-phenyl[(phenylacetyl)oxy]methyl 6,8-dioxa-2,4-diazadec-2-enoate (9)

was prepared following a literature procedure (Saulnier, et al. (1994). Bioorg Med Chem Lett 4, 1985-1990).

### 1,2,-dimethylethyl(S)-2-[(4-methylphenyl)sulfonyl]amino-4-[[bis[[[[(phenylacetyl)oxy]methoxy]carbony]amino]methylene]amino]pentanoate (10)

and 1,2,-dimethylethyl(S)-2-(acetyl)amino-4-[[bis[[[[(phenylacetyl)oxy]methoxy]carbonyl]amino]methylene]amino]pentanoate (11):

were prepared following a literature procedure (Saulnier, et al. (1994). Bioorg Med Chem Lett 4, 1985-1990).

10: ¹H NMR (500 MHz, CDCl₃): δ 1.23 (s, 9H), 1.57-1.62 (m, 1H), 1.71-1.77 (m, 3H), 2.39 (s, 3H), 3.45 (q, J=6.5, 2H), 3.68 (s, 2H), 3.71 (s, 2H), 3.77-3.79 (m, 1H), 5.23 (d, J=8.5 Hz, 1H), 5.80 (s, 2H), 5.82 (s, 2H), 7.24-7.36 (m, 12H), 7.71-7.73 (m, 2H), 8.29 (br t, J=5.3 Hz, 1H), 11.61 (br s, 1H).

11: ¹H NMR (500 MHz, CDCl₃): δ 1.46 (s, 9H), 1.56-1.71 (m, 3H), 1.86-1.90 (m, 1H), 2.02 (s, 3H), 3.42-3.48 (m, 2H), 3.67 (s, 2H), 3.71 (s, 2H), 4.51-4.54 (m, 1H), 5.80 (s, 2H), 5.82 (s, 2H), 6.19 (d, J=7.5 Hz, 1H), 7.26-7.36 (m, 10H), 8.31 (br t, J=5.5 Hz, 1H), 11.61 (br s, 1H).

### (S)-2-[(4-methylphenyl)sulfony]amino-4-[[bis[[[[(phenylacetyl)oxy]methoxy]carbonyl]amino]methylene]amino]pentanoic acid (12) and (S)-2-(acetyl)amino-4[[bis[[[[(phenylacetyl)oxy]methoxy]carbonyl]amino]methylene]amino]pentanoic acid (13)

were prepared following a literature procedure (Bryan, D. B. H., et al. (1977). J Am Chem Soc 99, 2353-2355). Briefly, each ester (10 and 11) was deprotected with 30% TFA in DCM at room temperature for 2 h and then purified by silica gel column chromatography with 2.5% MeOH in dichloromethane to give each acid 12 and 13 in 30-40% yield.

12: ¹H NMR (500 MHz, CDCl₃): δ1.66-1.75 (m, 3H), 1.83-1.89 (m, 1H), 2.40 (s, 3H), 3.36-3.46 (m, 2H), 3.68 (s, 2H), 3.71 (s, 2H), 4.01-4.05 (m, 1H), 5.48 (d, J=8.5 Hz, 1H), 5.78 (s, 2H), 5.82 (s, 2H), 7.24-7.35 (m, 12H), 7.72-7.74 (m, 2H), 8.32 (br t, J=5.3 Hz, 1H), 11.55 (br, 1H).

13: ¹H NMR (500 MHz, CDCl₃): δ 1.67-1.77 (m, 3H), 1.93-1.97 (m, 1H), 2.04 (s, 3H), 3.36-3.40 (m, 1H), 3.51-3.55 (m, 1H), 3.67 (s, 2H), 3.70 (s, 2H), 4.54-4.58 (m, 1H), 5.78 (s, 2H), 5.81 (s, 2H), 6.93 (d, J=7.5 Hz, 1H), 7.26-7.35 (m, 10H), 8.41 (br t, J=5.5 Hz, 1H), 11.63 (br, 1H).

### Methyl(S)-2-[(4-methylphenyl)sulfony]amino-4-[[bis[[[[(phenylacetyl)oxy]methoxy]carbonyl]amino]methylene]amino]pentanoate (14) and methyl(S)-2-(acetyl)amino-4-[[bis[[[[(phenylacetyl)oxy]methoxy]carbonyl]amino]methylene]amino]pentanoate (15)

were prepared following a literature procedure (Tangirala, R. S. A., et al. (2006). Bioorg Med Chem 14, 6202-6212). Briefly, each acid (12 and 13) was methylated with TMSCHN₂(2 M solution in hexane) in dry benzene at room temperature and then purified by silica gel column chromatography with 40% EtOAc in hexane for 14, 80% EtOAc in hexane for 15 to give each ester 14 and 15 in 40 to 45% yield, respectively.

14: ¹H NMR (500 MHz, CDCl₃): δ1.65-1.72 (m, 2H), 1.76-1.81 (m, 1H), 2.41 (s, 3H), 3.34 (q, J=6.5, 2H), 3.48 (s, 3H), 3.68 (s, 2H), 3.71 (s, 2H), 3.93-3.97 (m, 1H), 5.28 (d, J=9.0 Hz, 1H), 5.80 (s, 2H), 5.83 (s, 2H), 7.24-7.36 (m, 12H), 7.71-7.72 (m, 2H), 8.28 (br t, J=5.8 Hz, 1H), 11.61 (s, 1H). ¹³C NMR (125 MHz, CDCl₃): δ 21.5, 24.8, 30.2, 40.4, 40.8, 41.0, 52.6, 55.3, 80.5, 81.2, 127.2, 127.3, 127.5, 128.6, 128.7, 129.3, 129.4, 129.7, 132.7, 133.3, 136.5, 143.8, 152.4, 156.3, 162.1, 169.9, 170.3, 171.8, HRMS calcd for C₃₄H₃₉N₄O₁₂S (M+H)⁺727.2285. found 727.2280.

15: ¹H NMR (125 MHz, CDCl₃): δ 1.59-1.73 (m, 3H), 1.88-1.92 (m, 1H), 2.03 (s, 3H), 3.40-3.45 (m, 1H), 3.45-3.52 (m, 1H), 3.68 (s, 2H), 3.71 (s, 2H), 3.75 (s, 3H), 4.63-4.67 (m, 1H), 5.80 (s, 2H), 5.82 (s, 2H), 6.28 (d, J=8.0 Hz, 1H), 7.25-7.36 (m, 10H), 8.32 (br t, J=5.5 Hz, 1H), 11.61 (br s, 1H). ¹³C NMR (500 MHz, CDCl₃): δ 23.4, 25.4, 29.6, 40.6, 41.0, 41.2, 52.1, 52.8, 80.8, 81.4, 127.5, 127.8, 128.8, 129.0, 129.6, 129.7, 132.9, 133.5, 152.7, 156.5, 162.9, 170.2, 170.6, 172.9. HRMS calcd for C₂₉H₃₅N₄O₁₁ (M+H)⁺615.2302. found 615.2297.

### ProTAME Activation Analysis.

ProTAME was added to interphase Xenopus extract at 50 μM or cell growth media at 20 μM. For interphase extract, 800 μl of sample was collected at 0 min, 10 min, 20 min and 30 min after addition of proTAME and diluted to 8 ml with XB. For cell culture, approximately 800,000 cells were collected at 1 h, 2 h and 3 h after addition of proTAME and lysed in 400 μl lysis buffer as described above and subsequently diluted to 4 ml with lysis buffer. The diluted extract or cell lysate was extracted with 1.5 volume of ethyl acetate. The extracts were dried in vacuo and the dry extracts were resuspended in 200 μl of methanol for LC/MS analysis. LC/MS data were obtained using an Agilent series 1200 LC/6130 MS system with a reversed-phase C18 column (Phenomenex Luna C18(2), 4.6 mm×100 mm, 5 μm) and a CH₃CN/H₂O gradient solvent system beginning with 10% aqueous CH₃CN and ending at 100% CH₃CN at 20 min. 10 μl of each sample was injected for each analysis. The collected LC/MS profiles were further analyzed by extracting specific ions such as 343 (TAME) and 727 (proTAME) in the positive ion MS mode.

### Odyssey Scanner for Western Signal Quantification.

Secondary antibodies coupled to fluorophores (anti-mouse Alexa-Fluor 750 and anti-rabbit Alexa-Fluor680, Invitrogen) were used to detect and quantify signals from rabbit anti-Cdc20 (Santa-Cruz, sc-8358) and mouse anti-GAPDH (AbCam, ab8245) antibodies on the same membrane using an Odyssey (Li-Cor Biosciences) scanner. Quantifications are reported as CDC20/GAPDH signal ratio, normalized to control treatment.

### Cdc20/Cdh1 Binding Assay in HeLa Cells.

HeLa cells in DMEM 10% FBS were plated in T25 flasks at 20% confluence one day prior to the experiment. They were then synchronized by a double thymidine block (18 h for the first block, 8 h release and another 18 h for the second block, thymidine concentration: 2 mM). For analysis of Cdh1 binding, cells were released into 300 nM from the second thymidine block for 13 h and then washed into fresh medium. Six h later, cells were treated with 12 μM proTAME or proTAME or 0.06% DMSO for 2 h and then collected by trypin digrestion. For analysis of Cdc20 binding, cells were transfected with indicated siRNAs during the first release from thymidine block after two washes with DPBS (CellGro 21-030-CV) and addition of 6.3 ml OptiMEM. A volume (79 μl) of 20 μl Control siRNA#3 or a 1:1 mix of 20 μl MAD2 siRNA and BubR1 siRNA (Dharmacon D-004101-01, 5′-GGAAGAAGAUCUAGAUGUA_{UU}-3′ (SEQ ID NO: 11)) was mixed in 1381 μl OptiMEM in a tube, 23.7 μl OligoFectamine were mixed with 94.1 μl OptiMEM in a second tube. After 5 min incubation at RT, the tubes contents were mixed and siRNA-reagent complexes were allowed to form for 20 min at RT. The transfection mixes were added to cells in OptiMEM and FBS was added to 10% after 5 h transfection. Cyclin B1-4107 expressing adenovirus (1:100) was added at the start of the second thymidine block and kept in the medium for all subsequent steps. Cells were treated at 10 h after release with 100 nM okadaic acid, 25 μg/ml cycloheximide and 12 μM proTAME as indicated. After 2 h treatment, cells were collected by mitotic shake-off. Cell pellets were washed twice with DPBS and flash-frozen with liquid nitrogen and stored at −80° C. until use. Cell lysis and APC immunoprecipitation were performed as described above.

### Live Cell Imaging.

The imaging plate was mounted onto a motorized stage (Prior ProScan II) on a Nikon TE2000E PFS inverted microscope fitted with an incubation chamber maintained at 37° C. and supplied with 5% CO₂. A 20× Plan Apo 0.75 NA or 40× Plan Fluor 0.75 NA objective lens was used as indicated and images were collected with 2×2 binning DIC or GFP fluorescent images were taken every 12 min (unless otherwise specified) for 36 h with a Hamamatsu ORCA cooled CCD camera and Nikon Elements Software. TIFF files of each image were exported from Elements and used to build stacks and Quicktime movies with Metamorph imaging software (Molecular Devices). For manual analysis, mitotic duration is counted as the time between the first frame of chromosome condensation and the frame of chromosome segregation (anaphase) or decondensation (mitotic exit in the presence of nocodazole) or cell death (chromosomes shrinking to a small bright dot).

Emi1 Knockdown and proTAME Rescue.

HeLa H2B-GFP cells were plated in glass-bottom 24-well plates at 20% confluence one day prior to the experiment. Cells were transfected with a pool of Emi1 siRNA (Dharmacon M-012434-01, 5′-GAAAGGCUGUCAUGUAUUG-3′ (SEQ ID NO: 4); 5′-CAACAGACACUUAAUAGUA-3′ (SEQ ID NO: 5); 5′-CGAAGUGUCUCUGUAAUUA-3′ (SEQ ID NO: 6); 5′-GUACGAAGUGUCUCUGUAA-3′ (SEQ ID NO: 7)) or Control#3 siRNA (described above) at 18.5 nM with DharmaFect3. After 24 h, cells were treated with 0.06% DMSO or 12 μM proTAME. Live cell imaging was set up as described above.

### ProTAME Dose-Response.

HeLa H2B-GFP cells were plated in a 24-well plate in DMEM 10% FBS at 20% confluence one day prior to experiment and synchronized by double thymidine block as described above. ProTAME was added to final concentrations of 780 nM, 3 μM or 12 μM and proAAME was added to a final concentration of 12 μM at 8 h after release from the second thymidine block. 0.06% DMSO was used as the negative control. Live cell imaging was set up as described above.

### Exogenous Cyclin-GFP Expression, Live-Cell Imaging, and Quantitation.

HeLa H2B-RFP cells were transduced with cyclin B1-GFP or cyclin A2-GFP adenovirus for 40 h. Phenol Red-Free DMEM (Mediatech) supplemented with 10% FBS and 1:100 Penicillin-Streptomycin-Glutamine (Mediatech) was used as imaging medium. 20 μM proTAME, 20 μM proTAME or 150 nM nocodazole was added 45 min prior to the start of imaging. Live cell imaging was set up as described above except that the cells were plated in an 8-well chambered coverglasses (NUNC Lab-tek 155411). Four positions per treatment group were imaged with DIC transmitted light, red fluorescence, and green fluorescence (Semrock GFP/HcRed “Pinkel” filter set) at 12 min intervals for 24 h. Stacks of red and green fluorescence were merged, saved as AVI video files and analyzed using ImageJ. For quantitation, the first GFP-positive cells that undergo mitosis in three separate movies were chosen, giving at least 30 cells quantitated for each treatment group. Mean intensity values for the green channel were collected for a cytoplasmic region of a cell upon mitotic entry, mitotic exit, or after 1 h of mitotic arrest. At the same time points, background mean green intensity was determined and individually subtracted from the cytoplasmic mean intensity. This background corrected mean intensity value was then used to determine the percentage of the original GFP signal remaining at the completion of division (for the control cells) or after 1 h of mitotic arrest (for the nocodazole and proTAME treated cells). The average values for all quantitated cells were plotted with the error bars representing standard error of the mean.

### Immunofluorescence.

HeLa cells grown in DMEM+10% FBS were plated on 25 mm glass coverslips in a 6-well dish at a density of 130,000/ml×3 ml 48 h prior to treatment. They were then treated with 0.06% DMSO, 12 μM proTAME, 300 nM nocodazole or 300 nM taxol for 2 h. The cells were washed twice with PBS and fixed with 3% paraformaldehyde for 15 min. The cells were then washed with PBS and permeabilized with PBS plus 0.5% Triton X-100 for 2 min. The cells were then washed with PBS and blocked with PBS plus 5% FBS for 1 h. CREST antisera diluted 1:50 into PBS was added to the cells and incubated at room temperature for 1 h. The cells were washed with PBS and incubated with 1:1000 anti-human-Alexa 568 (Invitrogen, A21090) and 1:100 anti-α-tubulin-FITC for 1 h. The cells were then washed with PBS and the nuclei were stained with 1 μg/ml Hoechst 33342. The cover slips were mounted in 0.1M N-propylgallate in 9:1 glycerol:PBS. Z-series images were taken on a Nikon TE2000 microscope with PerkinElmer spinning disk confocal device. Maximal Z-projection images of individual cells were made by Image J. To measure interkinetochore distances, a straight line was drawn across a kinetochore pair in the same confocal plane and pixel intensities along the line were plotted so that each kinetochore would be represented by a peak on the line. The interkinetochore distance was calculated as the distance between the peaks. Fifty-five kinetochore pairs from 5 cells treated with DMSO or proTAME were measured and the p-value was calculated with a paired student test.

### Mad2 Knockdown and Time Point Analysis.

HeLa H2B-GFP cells were plated in a 24-well plate in DMEM+10% FBS at 20% confluence one day prior to the experiment and synchronized by double thymidine block as described above. The cells were released from the first thymidine block into 200 μl OptiMem without FBS. Transfection was performed immediately after the first thymidine release. To prepare the transfection mixture for one well, 40 μl OptiMem was mixed with 2.5 μl of 20 μM Mad2 siRNA stock (GGAACAACUGAAAGAUUGGdTdT (SEQ ID NO: 8), synthesized by DHARMACON) or control (D-001210-01-20, DHARMACON), and 6.5 μl of OptiMem was mixed with 1 μl of Oligofectamine (Invitrogen, 12252-011). The two mixtures were left at room temperature for 5 min before being mixed together and incubated for additional 20 min and then added to the cells to a final volume of 250 μl. 4 h after transfection, 250 μl of DMEM+20% FBS were added to cells. 8 h after the release, 500 μl of 4 mM thymidine in DMEM+10% FBS was added to each well to make the final concentration of 2 mM and the cells were incubated for another 18 h before being released into growth medium. At 8 h after release, cells were treated 0.06% DMSO, 12 μM proTAME, 300 nM nocodazole or 12 μM proTAME plus 300 nM nocodazole in growth medium. Cell samples were collected at 4 h, 8 h, 10 h, 12 h, 14 h, 16 h and 20 h post-release and protein levels were analyzed by Western blot.

Cdc20 Knockdown Sensitization to proTAME Treatment.

HeLa H2B-GFP cells were plated in glass-bottom 24-well plates (Greiner Bio-One 662892) at 20% confluence one day prior to the experiment. Cells were transfected with DharmaFect3, following the manufacturer's protocol at a final concentration of 18.5 nM control siRNA#3 or a mix of 1.85 nM Cdc20 siRNA completed to 18.5 nM with control siRNA#3. After 24 h transfection, cells were treated with DMSO or 4 μM proTAME and live cell imaging was set up immediately as described above.

### UbCH10 and Cdc27 Knockdown and Hesperadin Treatment.

HeLa H2B-GFP cells were plated in glass-bottom 24-well plates at 20% confluence one day prior to the experiment and synchronized by double thymidine block as described above. Cells were transfected with UbcH10 siRNA (Dharmacon D-004693-15, 5′-UAAAUUAAGCCUCGGUUGA_{UU}-3′ (SEQ ID NO: 9)), Cdc27 siRNA (Dharmacon J-003229-11, 5′-GGAAAUAGCCGAGAGGUAA_{UU}-3′ (SEQ ID NO: 10)) or Control#3 siRNA (described above) at 18.5 nM with DharmaFect3, during the release from the first thymidine block. Cells were treated with 100 nM Hesperadin or DMSO 8 h after release from the second thymidine block and live cell imaging was set up as described above.

### Measuring Cycloheximide-Sensitivity of Drug-Induced Arrest.

HeLa H2B-GFP cells were plated in a 24-well plate in DMEM+10% FBS at 20% confluence one day prior to experiment and synchronized by double thymidine block as synchronized above. At 8 h after release from the second block, cells were treated with 12 μM proTAME, 300 nM nocodazole or 150 nM taxol and 4 h later, cells were left untreated or treated with an addition of 25 μg/ml cycloheximide. Live cell imaging was set up as described above.

### Measuring Hesperadin-Sensitivity of Drug-Induced Arrest.

HeLa H2B-GFP cells were plated in a 24-well plate in DMEM+10% FBS at 20% confluence one day prior to experiment and synchronized by double thymidine block as described above. At 8 h after release from the second block, 100 nM hesperadin, 12 μM proTAME with or without 100 nM hesperadin, 300 nM nocodazole with or without 100 nM hesperadin or 150 nM taxol with or without 100 nM hesperadin were added to cells. Untreated cells were used as the control. Live cell imaging was set up as described above. For experiments with MG132, at 10 h after release from the second block, 3 μM MG132, or 3 μM MG132 plus 100 nM hesperadin, or 3 μM MG132 plus 100 nM hesperadin and 12 μM proTAME were added to cells. Alternatively, at 10 h after release from the second block, 10 μM MG132 with or without 25 μg/ml cycloheximide was added to the cells. 30 min after, cells were left untreated or treated with 100 nM hesperadin or 100 nM hesperadin and 12 μM proTAME. Live cell imaging was set up as described above.

### Measuring Mad2-Dependence of MG132-Induced Arrest.

HeLa H2B-GFP cells were plated in a 24-well plate in DMEM+10% FBS at 20% confluence one day prior to experiment and synchronized by double thymidine block as described above. Mad2 siRNA transfection was performed as described above. At 10 h after release from the second block, 10 μM MG132 with or without 25 μg/ml cycloheximide was added to the cells. Live cell imaging was set up as described above. Manual analysis was focused only on cells that entered mitosis after MG132 addition.

### Chromosome Congression Analysis.

HeLa H2B-GFP cells were plated in 35 mm glass-bottom dishes (MatTek) in DMEM+10% FBS at 20% confluence one day prior to the experiment and synchronized by double thymidine block as described above. Drugs were added as follows to a final volume of 3 ml from 2× concentrated preparation in culture medium. DMSO (0.06%), proTAME (3 and 12 μM) or 10 nM Nocodazole were added 8 h after release from the second block, while MG132 (10 μM) was added at 10 h. H2B-GFP was imaged for 4 hrs every 3 min at 40× magnification as described above.

### Click-iT Chemistry Labeling of De Novo-Translated Proteins.

HeLa H2B-GFP cells (600,000) were plated in 3 mL DMEM+10% FBS in 6-well plates 24 h prior to synchronization. Cells were arrested in interphase by 2 mM Thymidine treatment for 24 h. To label proteins translated in S/G2 phase, three hours after release from thymidine-block, the cells were washed once with warm DPBS with Mg²⁺/Ca²⁺ and switched to filter-sterilized labeling medium (Methionine-free medium from Sigma, catalog #D0422, supplemented with 10 mL FBS pre-dialyzed against 1L DPBS, 2 mM Glutamine and 568 μM L-Cysteine). After 30 min pre-incubation to deplete the remaining intracellular pool of Methionine, the methionine analog L-azidohomoalanine (AHA) was added at 250 μM (Invitrogen, catalog #C10102) and the cells were incubated for 3 h in the presence or absence of 25 μg/mL cycloheximide. To label proteins translated in mitosis, cells were treated with 300 nM nocodazole or 12 μM proTAME at 5 h after release from thymidine block and allowed to enter mitosis. Mitotic cells were collected by mitotic shake off, washed once in warm DPBS with Mg²⁺/Ca²⁺ and switched to labeling medium. After 30 min pre-incubation, 250 μM AHA was added. Labeling was allowed to occur for 12 h in the presence or absence of 25 μg/mL cycloheximide. After labeling, the cells were collected by trypsinization (interphase cells) or mitotic shake-off (mitotic cells), washed twice with DPBS with Mg2+/Ca2+ and lysed in 50 μL lysis buffer (Tris-HCl 50 mM pH 8.0, SDS 1% supplemented with 250 U/mL Benzonase, VWR, catalog #80108-806 and EDTA-free protease inhibitors, Roche). After 15 min on ice, cells were vortexed and centrifuged at 15,000 g at 4 C for 5 min. Supernatants were collected and protein concentrations were determined using the BCA assay (Pierce). Proteins (200 μg) were labeled with biotin-azide following the manufacturer's protocol (Invitrogen, catalog #B10184) and the protein reaction buffer kit (catalog #C10276). Labeled proteins were desalted with desalting columns (Thermo Scientific, catalog #89889) pre-washed with incubation buffer (NP-40 1%, SDS 0.1% in DPBS with Ca2+/Mg2+, with protease inhibitors). Ten percent of proteins were kept aside as total protein control for western blots of specific proteins, and another 10% were conserved to run Streptavidin-HRP western blots to detect all labeled proteins. The remaining sample was incubated at room temperature with Neutravidin agarose resin pre-washed with incubation buffer (Thermo scientific, catalog #29200) to purify biotin-labeled proteins. The resin was washed once with incubation buffer and three times with wash buffer (NP-40 1% in DPBS with Ca2+/Mg2+, with protease inhibitors). Purified proteins were boiled in SDS-PAGE loading buffer and tested by western blotting.

### Statistical Analysis.

For each indicated figure and conditions, the data sample size (N), median and average values are reported. Statistical analysis was performed using the software Jmp 8.0 (SAS Institute Inc.). Samples were compared two by two using the Mann-Whitney-Wilcoxon non-parametric statistical test. The p values are reported. The samples were considered statistically significantly different when p was inferior to 0.05. Very small p values were reported as zero.

TABLE 1
Statistical Analyses

| | | | | | | | | | MW-Wilcoxon |
|---|---|---|---|---|---|---|---|---|---|
| | | | | | | | | | Statistical |
| | | | Median | Average | | | Median | Average | test |
| Figure | Condition 1 | N1 | (min) | (min) | Condition 2 | N2 | (min) | (min) | p value |
| 4E_ProTAME | DMSO | 150 | 612 | 625.0 | proTAME | 150 | 696 | 712.8 | 0 |
| (12 μM)- | | | | | at 0 hr | | | | |
| induced delay | | | | | proTAME | 150 | 660 | 669.0 | 3.97E−11 |
| in mitotic entry | | | | | at 2 hrs | | | | |
| correlates with | | | | | proTAME | 150 | 624 | 636.6 | 0.07 |
| the time of | | | | | at 4 hrs | | | | |
| addition after | | | | | proTAME | 150 | 612 | 619.4 | 0.88 |
| release from | | | | | at 6 hrs | | | | |
| thymidine | | | | | proTAME | 150 | 612 | 622.6 | 0.96 |
| block | | | | | at 8 hrs | | | | |
| 5A_ProTAME | DMSO | 150 | 96 | 121.4 | proTAME | 150 | 156 | 165.4 | 1.43E−12 |
| induced mitotic | | | | | 780 nM | | | | |
| arrest in HeLa | | | | | proTAME | 150 | 348 | 444.6 | 0 |
| cells | | | | | 3 μM | | | | |
| | | | | | proTAME | 150 | 1680 | 1640.5 | 0 |
| | | | | | 12 μM | | | | |
| | | | | | proAAME | 150 | 84 | 100.5 | 0.42 |
| | | | | | 12 μM | | | | |
| 5B_Partial | Control siRNA | 153 | 60 | 72.8 | Cdc20 siRNA | 150 | 96 | 144.2 | 0 |
| Cdc20 knockdown | DMSO | | | | DMSO | | | | |
| and low proTAME | | | | | Control siRNA | 114 | 288 | 496.5 | 0 |
| concentration | | | | | proTAME | | | | |
| synergize in | Cdc20 siRNA | 150 | 96 | 144.2 | Cdc20 siRNA | 112 | 1170 | 1235.3 | 0 |
| delaying mitosis | DMSO | | | | proTAME | | | | |
| in HeLa cells | | | | | | | | | |
| | | | | | | | | | MW-Wilcoxon |
| | | | Median | Average | | | Median | Average | Statistical |
| | | | (fraction | (fraction | | | (fraction | (fraction | test |
| Figure | Condition 1 | N1 | remaining) | remaining) | Condition 2 | N2 | remaining) | remaining) | p value |
| 5D_ProTAME | proAAME | 31 | 0.15 | 0.15 | Nocodazole | 34 | 0.83 | 0.87 | 3.87E−08 |
| stabilizes | | | | | proTAME | 30 | 1.17 | 1.17 | 2.07E−11 |
| CyclinB1 | Nocodazole | 34 | 0.83 | 0.87 | proTAME | 30 | 1.17 | 1.17 | 7.86E−12 |
| 5D_ProTAME | proAAME | 40 | 0.06 | 0.08 | Nocodazole | 37 | 0.28 | 0.28 | 4.71E−14 |
| stabilizes | | | | | proTAME | 30 | 0.95 | 0.99 | 1.12E−12 |
| CyclinA2 | Nocodazole | 37 | 0.28 | 0.28 | proTAME | 30 | 0.95 | 0.99 | 7.38E−06 |
| | | | | | | | | | MW-Wilcoxon |
| | | | | | | | | | Statistical |
| | | | Median | Average | | | Median | Average | test |
| Figure | Condition 1 | N1 | (mm) | (mm) | Condition 2 | N2 | (mm) | (mm) | p value |
| 5E_ProTAME | DMSO | 55 | 1.22 | 1.23 | proTAME | 55 | 1.28 | 1.26 | 0.23 |
| does not | | | | | | | | | |
| alter inter- | | | | | | | | | |
| kinetochore | | | | | | | | | |
| distance | | | | | | | | | |
| | | | | | | | | | MW-Wilcoxon |
| | | | | | | | | | Statistical |
| | | | Median | Average | | | Median | Average | test |
| Figure | Condition 1 | N1 | (min) | (min) | Condition 2 | N2 | (min) | (min) | p value |
| 6A_ProTAME | Control siRNA | 150 | 108 | 121.7 | MAD2 siRNA | 150 | 12 | 13.2 | 0 |
| arrest is MAD2- | DMSO | | | | DMSO | | | | |
| dependent | | | | | Control siRNA | 150 | 1812 | 1680.1 | 0 |
| | | | | | Nocodazole | | | | |
| | | | | | Control siRNA | 150 | 1488 | 1512.4 | 0 |
| | | | | | proTAME | | | | |
| | MAD2 siRNA | 150 | 12 | 13.2 | MAD2 siRNA | 150 | 36 | 98.2 | 0 |
| | DMSO | | | | Nocodazole | | | | |
| | | | | | MAD2 siRNA | 150 | 84 | 121.7 | 0 |
| | | | | | proTAME | | | | |
| 6C_ProTAME | DMSO | 150 | 102 | 110.9 | DMSO | 150 | 96 | 98.7 | 0.05 |
| arrest is | | | | | Hesperadin | | | | |
| hesperadin- | Taxol | 150 | 2070 | 1946.9 | Taxol | 150 | 156 | 168.7 | 0 |
| sensitive | | | | | Hesperadin | | | | |
| | proTAME | 150 | 1272 | 1329.3 | proTAME | 151 | 228 | 262.2 | 0 |
| | | | | | Hesperadin | | | | |
| | Nocodazole | 150 | 2094 | 1851.8 | Nocodazole | 150 | 552 | 579.4 | 0 |
| | | | | | Hesperadin | | | | |
| 6D_UbcH10 | Control siRNA | 150 | 96 | 134.5 | Control siRNA | 150 | 120 | 115.6 | 0.22 |
| and Cdc27 | | | | | Hesperadin | | | | |
| siRNA induced | | | | | UbcH10 siRNA | 150 | 156 | 330.5 | 1.51E−10 |
| mitotic delays | | | | | Cdc27 siRNA | 150 | 276 | 575.6 | 0 |
| are hesperadin- | UbcH10 siRNA | 150 | 156 | 330.5 | UbcH10 siRNA | 150 | 108 | 115.8 | 5.20E−10 |
| sensitive | | | | | Hesperadin | | | | |
| | Cdc27 siRNA | 150 | 276 | 575.6 | Cdc27 siRNA | 150 | 132 | 131.8 | 0 |
| | | | | | Hesperadin | | | | |
| 7A_MG132 | Control siRNA | 169 | 840 | 830.1 | MAD2 siRNA | 150 | 667.5 | 605.3 | 3.81E−05 |
| arrest is MAD2- | 3 μM MG132 | | | | 3 μM MG132 | | | | |
| dependent | | | | | | | | | |
| 7B_Hesperadin | MG132 3 μM | 150 | 174 | 502.2 | MG132 3 μM | 154 | 1440 | 1549.9 | 0 |
| overrides | Hesperadin | | | | DMSO | | | | |
| MG132 3 μM | | | | | MG132 3 μM | 150 | 1296 | 1334.0 | 0 |
| arrest in HeLa | | | | | Hesperadin | | | | |
| cells | | | | | proTAME | | | | |
| 7C_Taxol does | MG132 3 μM | 151 | 420 | 705.3 | MG132 3 μM | 155 | 312 | 511.4 | 0 |
| not restore | Hesperadin | | | | Hesperadin | | | | |
| mitotic arrest in | Taxol | | | | MG132 3 μM | 150 | 1524 | 1598.9 | 0 |
| the presence of | | | | | Hesperadin | | | | |
| MG132 3 μM | | | | | proTAME | | | | |
| and hesperadin | | | | | | | | | |
| 7D_Hesperadin | MG132 10 μM | 149 | 2160 | 2185.3 | MG132 10 μM | 150 | 2172 | 2169.8 | 0.95 |
| overrides | Hesperadin | | | | DMSO | | | | |
| MG132 10 μM | | | | | MG132 10 μM | 119 | 2724 | 2657.5 | 1.35E−14 |
| arrest in HeLa | | | | | Hesperadin | | | | |
| cells | | | | | proTAME | | | | |
| 8A_Cycloheximide | Nocodazole | 70 | 2160 | 1777.0 | Nocodazole | 70 | 444 | 442.5 | 0 |
| sitivity of mitotic | | | | | Cycloheximide | | | | |
| arrests. | Taxol | 100 | 1602 | 1557.6 | Taxol | 100 | 516 | 538.6 | 0 |
| | | | | | Cycloheximide | | | | |
| | proTAME | 100 | 972 | 1052.0 | proTAME | 100 | 1608 | 1577.9 | 0 |
| | | | | | Cycloheximide | | | | |
| 8B_MG132 arrest is | Control siRNA | 66 | 1614 | 1715.1 | Control siRNA | 81 | 1788 | 1897.2 | 0.03 |
| MAD2-dependent | MG132 10 μM | | | | MG132 10 μM | | | | |
| (see cell fate | | | | | Cycloheximide | | | | |
| distribution in | MAD2 siRNA | 64 | 1716 | 1755.2 | MAD2 siRNA | 54 | 834 | 1078.0 | 3.11E−05 |
| figure) | MG132 10 μM | | | | MG132 10 μM | | | | |
| | | | | | Cycloheximide | | | | |
| 8C_Hesperadin | MG132 10 μM | 133 | 1104 | 1335.9 | MG132 10 μM | 151 | 2520 | 2346.4 | 0 |
| rapidly overrides | Cycloheximide | | | | DMSO | | | | |
| 10 μM MG132 arrest | Hesperadin | | | | Cycloheximide | | | | |
| in the presence of | | | | | MG132 10 μM | 132 | 2532 | 2383.9 | 0 |
| cycloheximide | | | | | Cycloheximide | | | | |
| | | | | | Hesperadin | | | | |
| | | | | | proTAME | | | | |
| S4A_ProTAME | DMSO | 26 | 24 | 30.9 | proAAME | 31 | 36 | 32.1 | 0.25 |
| treatment arrests | | | | | 12 μM | | | | |
| hTERT-RPE1 cells | | | | | proTAME | 24 | 522 | 819.0 | 8.15E−10 |
| in mitosis. | | | | | 6 μM | | | | |
| S4C_ProTAME | Untreated | 96 | 21 | 21.5 | proTAME | 93 | 21 | 21.7 | 0.66 |
| induces a mild delay | | | | | 3 μM | | | | |
| in chromosome | | | | | proTAME | 95 | 24 | 26.0 | 1.37E−09 |
| congression | | | | | 12 μM | | | | |
| | | | | | MG132 10 μM | 52 | 21 | 21.6 | 0.86 |
| | | | | | Nocodazole | 91 | 24 | 26.2 | 7.76E−08 |
| | | | | | 10 nM | | | | |
| S5G_Hesperadin | Release in | 150 | 96 | 124.4 | Release in | 144 | 1410 | 1516.4 | 0 |
| overrides 12 μM | medium | | | | proTAME | | | | |
| proTAME-induced | | | | | 12 μM | | | | |
| mitotic arrest after | | | | | Release in | 150 | 156 | 202.1 | 1.05E−09 |
| washout from a | | | | | proTAME | | | | |
| nocodazole-induced | | | | | 12 μM | | | | |
| mitotic arrest | | | | | Hesperadin | | | | |
| | | | | | 100 nM | | | | |
| | Release in | 144 | 1410 | 1516.4 | Release in | 150 | 156 | 202.1 | 0 |
| | proTAME | | | | proTAME | | | | |
| | 12 μM | | | | 12 μM | | | | |
| | | | | | Hesperadin | | | | |
| | | | | | 100 nM | | | | |

### Example 5

### ProTAME Synergy

To determine whether proTAME treatment can enhance mitotic arrest and cell death induced by taxol or a proteasome inhibitor (MG132), HeLa H2B-GFP cells were co-treated with low doses of the microtubule poison Taxol or the proteasome inhibitor MG132 in combination with a low dose of proTAME.

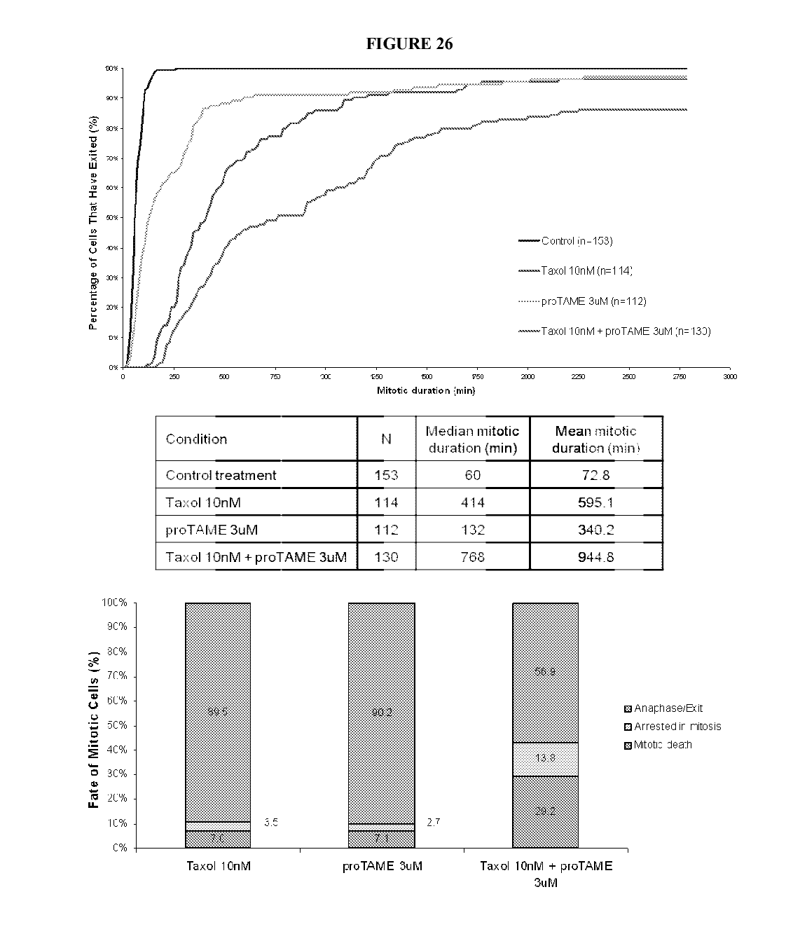

Unsychronized HeLa H2B-GFP cells were treated with DMSO (control), taxol 10 nM or proTAME 3 μM or a combination of Taxol 10 nM and proTAME 3 μM (FIG. 26). The cells were imaged by FITC fluorescence microscopy to monitor the cell cycle and mitotic events. Duration of mitotic events, initiated during the first 21 hours of imaging, was manually determined for indicated number of cells and the cumulative percentage of cells was plotted as a function of mitotic duration. Individual chemicals induced a mitotic delay with a median of 414 min for Taxol alone and 132 min for proTAME alone as compared to a median mitotic duration of 60 min for control-treated cells. The combination resulted in a synergistic increase in mitotic duration to 768 min median mitotic duration. Single treatments resulted in about 10% of cells that remained arrested or died in mitosis throughout the 48 hrs movie while most cells exited mitosis. In contrast, 43% cells stayed arrested or died in mitosis in the co-treatment, therefore indicating synergism in inducing mitotic arrest and mitotic cell death.

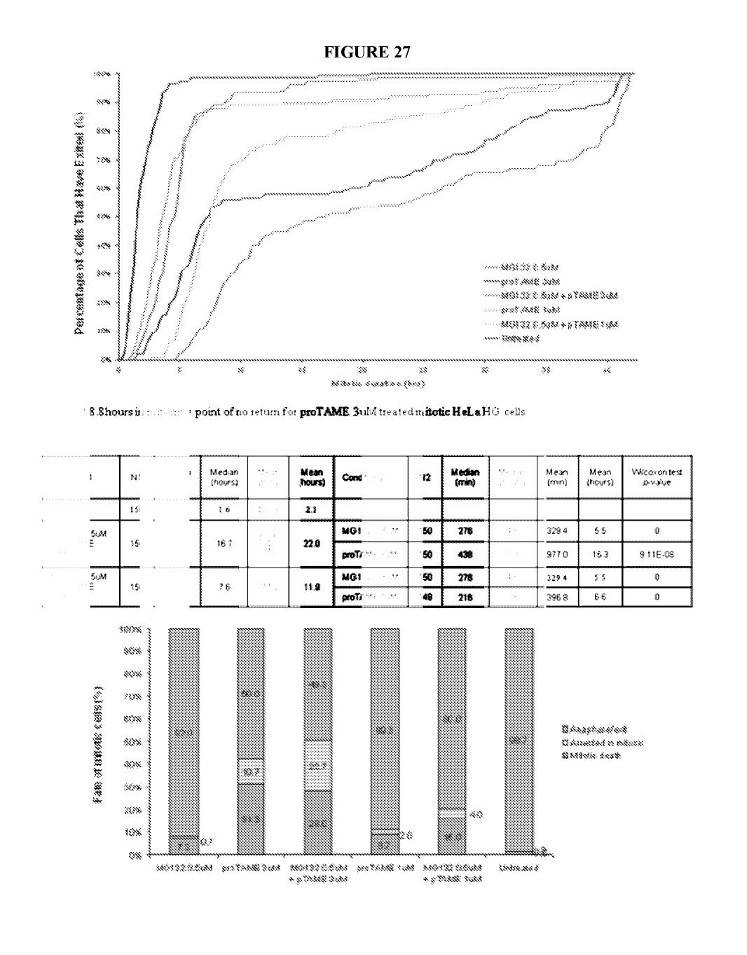

To analyze interactions between proTAME and a proteasome inhibitor (FIG. 27), HeLa H2B-GFP cells were synchronized by double thymidine block (2 mM thymidine block for 18 hrs, release in medium for 8 hrs 2 mM thymidine block for 18 hrs), and treated with the drugs 10 hrs after release from the block as the cells just entered mitosis. In cells treated with a single drug, MG132 induced a mitotic delay of 276 min (median) while proTAME (3 μM) induced a delay of 438 minutes. The effect of co-treatment was synergistic, resulting in a median mitotic arrest of more than 1000 min. The synergistic effect in duration of mitotic arrest was also observed with a lower dose of proTAME (1 μM) where the cells arrested for a median time of 216 min while the combination with MG132 0.5 μM resulted in a median arrest of 456 min. The co-treatments resulted in an increase in the percentage of cells indefinitely arrested in mitosis or dying in mitosis during the movie, as compared to single treatments, indicating a benefit in terms of mitotic phenotype of the combination.

These data demonstrate that co-treatment of cells with the APC/C inhibitor, proTAME, and drugs that stimulate the spindle assembly checkpoint (taxol) or inhibit proteasome-dependent degradation (MG132) results in prolonged mitotic arrest associated with increased mitotic cell death. These co-treatments are beneficial in terms of increased mitotic duration and potency to induce cell death in mitosis in development of anticancer co-treatment regimens.

### OTHER EMBODIMENTS

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A composition comprising a prodrug of tosyl-L-arginine methylester (TAME), wherein said compound diffuses across the plasma membrane of a cell.

2. The composition of claim 1, comprising a TAME derivative in which a guanidine is linked to a protecting group.

3. The composition of claim 1, wherein said prodrug comprises a TAME derivative in which a guanidine group is protected by a carbamate group.

4. The composition of claim 1, wherein said prodrug comprises an esterase-activatable N,N′-bis(acyloxymethyl carbamate) derivative of TAME.

5. The composition of claim 1, wherein said prodrug is characterized as having a eukaryotic cell permeability level at least 20% greater than that of TAME.

6. The composition of claim 1, wherein said compound is proTAME:

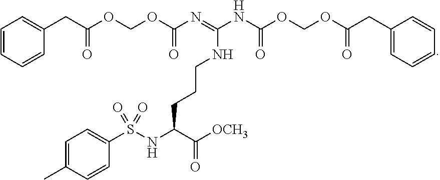 [SMILES: COC(=O)[C@H](CCCN/C(=N\C(=O)OCOC(=O)Cc1ccccc1)NC(=O)OCOC(=O)Cc1ccccc1)NS(=O)(=O)c1ccc(C)cc1]

7. A pharmaceutical composition comprising the compound of claim 1.

8. The composition of claim 1, wherein said cell is eukaryotic, mammalian, or human.

9. The composition of claim 1, wherein said compound inhibits an activity of an anaphase promoting complex (APC).

10. The composition of claim 9, wherein said compound contacts a component of a tetratricopeptide repeats (TPR) subcomplex of an APC.

11. The composition of claim 10, wherein said component of a TPR subcomplex is APC3/Cdc27, APC6, APC7, or APC8.

12. The composition of claim 1, wherein said compound induces a cell cycle checkpoint.

13. The compound of claim 12, wherein said cell cycle checkpoint is the spindle assembly checkpoint (SAC).

14. A formulation comprising an amount of a prodrug of tosyl-L-arginine methylester (TAME) that is sufficient to inhibit the degradation of a substrate of an anaphase-promoting complex/cyclosome (APC) for arresting the mitotic cycle of a cell.

15. The formulation of claim 9, further comprising a pharmaceutical carrier.

16. The formulation of claim 9, further comprising a spindle assembly checkpoint activator or an inhibitor of proteasome-dependent degradation.

17. The formulation of claim 16, wherein the spindle assembly checkpoint activator is paclitaxol or Taxol™.

18. The formulation of claim 16, wherein the inhibitor of proteasome-dependent degradation is MG132.

19. The formulation of claim 9, wherein prodrug contacts the cell in vivo, in vitro, or ex vivo.

20. The formulation of claim 9, wherein the prodrug increases the median or mean mitotic duration of the cell.

21. The formulation of claim 9, wherein the prodrug induces death of the cell.

22. The formulation of claim 9, further comprising administering a therapeutic agent.

23. The formulation of claim 22, wherein the therapeutic agent comprises a chemotherapy, a radiation therapy, an immunotherapy, or a hormone therapy.

24. The formulation of claim 23, wherein the radiation therapy is actinium-225 (Ac²²⁵), bismuth-213 (Bi²¹³), boron-10 (B¹⁰)+neutron therapy, holmium-166 (Ho¹⁶⁶), iodine-125 (I¹²⁵), iodine-131 (I¹³³), iridium-192 (Ir¹⁹²), lead-212 (Pb²¹²), lutetium-177 (Lu¹⁷⁷), rhenium-186 (Re¹⁸⁶), samarium-153 (Sm¹⁵³), strontium-89 (Sr⁸⁹), or yttrium-90 (Y⁹⁰).

25. The formulation of claim 23, wherein the immunotherapy is an antibody selected from the group consisting of rituximab (Rituxan®), trastuzumab (Herceptin®), gemtuzumab ozogamicin (Mylotarg®), alemtuzumab (Campath®), ibritumomab tiuxetan (Zevalin®), tositumomab (Bexxar®), cetuximab (Erbitux®), bevacizumab (Avastin®), panitumumab (Vectibix®), ofatumumab (Arzerra®), denosumab (Xgeva™), ipilimumab (Yervoy™), and brentuximab vedotin (Adcetris™).

26. The formulation of claim 23, wherein the hormone therapy is tamoxifen (Nolvadex®), an aromatase inhibitor, anastrozole (Arimidex®), letrozole (Femara®), or fulvestrant (Faslodex®).

27. The formulation of claim 23, wherein the chemotherapy is carboplatin (Paraplatin), cisplatin (Platinol, Platinol-AQ), cyclophosphamide (Cytoxan, Neosar), doxorubicin (Adriamycin), etoposide (VePesid), fluorouracil (5-FU), gemcitabine (Gemzar), irinotecan (Camptosar), methotrexate, (Folex, Mexate, Amethopterin), paclitaxel (Taxol), topotecan (Hycamtin), vincristine, (Oncovin, Vincasar PFS), or vinblastine (Velban).

28. The formulation of claim 9, wherein said cell is characterized by a proliferative disorder.

29. The method of claim 28, wherein the cell proliferative disorder is cancer, Castleman Disease, Gestational Trophoblastic Disease, or myelodysplastic syndrome.

30. The formulation of claim 29, wherein the cancer is adrenal cortical cancer, anal cancer, bile duct cancer, bladder cancer, bone cancer, brain or a nervous system cancer, breast cancer, cervical cancer, colon cancer, rectral cancer, colorectal cancer, endometrial cancer, esophageal cancer, Ewing family of tumor, eye cancer, gallbladder cancer, gastrointestinal carcinoid cancer, gastrointestinal stromal cancer, Hodgkin Disease, intestinal cancer, Kaposi Sarcoma, kidney cancer, large intestine cancer, laryngeal cancer, hypopharyngeal cancer, laryngeal and hypopharyngeal cancer, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, lung cancer, non-small cell lung cancer, small cell lung cancer, lung carcinoid tumor, lymphoma, lymphoma of the skin, malignant mesothelioma, multiple myeloma, nasal cavity cancer, paranasal sinus cancer, nasal cavity and paranasal sinus cancer, nasopharyngeal cancer, neuroblastoma, non-Hodgkin lymphoma, oral cavity cancer, oropharyngeal cancer, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma, adult soft tissue sarcoma, skin cancer, basal cell skin cancer, squamous cell skin cancer, basal and squamous cell skin cancer, melanoma, stomach cancer, small intestine cancer, testicular cancer, thymus cancer, thyroid cancer, uterine sarcoma, uterine cancer, vaginal cancer, vulvar cancer, Waldenstrom Macroglobulinemia, or Wilms Tumor.

31. The formulation of claim 29, wherein the cancer is primary or metastatic.

32. The formulation of claim 29, wherein the cancer occurs in a child or an adult.
